(19) United States
(12) Patent Application Publication
(10) Pub. No.: US 2011/0171260 A1
(43) Pub. Date: Jul. 14, 2011

(54) INFLUENZA DNA VACCINATION AND METHODS OF USE THEREOF

(75) Inventors: SRINIVAS RAO, COLUMBIA, MD (US); GARY J. NABEL, WASHINGTON, DC (US); ZHI-YONG YANG, POTOMAC, MD (US); CHIH-JEN WEI, GAITHERSBURG, MD (US); WING-PUI KONG, GERMANTOWN, MD (US)

(73) Assignee: THE UNITED STATES OF AMERICA, AS REPRESENTED BY THE SECRETARY, DEPARTMENT OF HEALTH AND HUMAN SER., BETHESDA, MD (US)

(21) Appl. No.: 12/838,292

(22) Filed: Jul. 16, 2010

Related U.S. Application Data
(63) Continuation-in-part of application No. PCT/US2009/031329, filed on Jan. 16, 2009.
(60) Provisional application No. 61/023,341, filed on Jan. 24, 2008.
(60) Provisional application No. 61/021,586, filed on Jan. 16, 2008.

(51) Int. Cl. A61K 39/145; C12N 15/63; A61P 31/16

(52) U.S. Cl. 424/209.1; 435/320.1

(57) ABSTRACT

The present invention relates to an influenza immunogen that includes one or more DNA constructs encoding at least two divergent influenza HAs, wherein each of such one or more DNA constructs encodes one or more of the at least two divergent influenza HAs. Such an immunogen, when administered to a subject, induces an immune response to a plurality of strains of influenza virus, wherein at least one strain of the plurality of strains does not encode any of the divergent influenza HAs encoded by the immunogen. The divergent influenza HAs can be swine influenza HAs or equine influenza HAs, such as influenza H1 HAs or influenza H3 HAs. The invention also relates to a method to use such an immunogen to induce such an immune response as well as to DNA constructs comprising such divergent influenza HAs. Such an immunogen can provide a heterologous as well as a homologous immune response. Such an immunogen can be used to induce an immune response against evolving influenza virus.

This application is a U.S. continuation-in-part application under 35 U.S.C. 111 and claims the benefit of PCT Application PCT/US2009/031329, filed Jan. 16, 2009, which claims priority to U.S. Provisional Application No. 61/021,586, filed Jan. 16, 2008, and U.S. Provisional Application No. 61/023,341, filed Jan. 24, 2008, all of which are hereby expressly incorporated by reference in their entireties.

### FIELD OF THE INVENTION

Aspects of the present invention concern one or more DNA constructs encoding influenza hemagglutinin (HA) proteins, immunogens and vaccines containing said one or more DNA constructs and use of these compositions to induce an immune response and/or to protect subjects against infection with avian, swine, and equine influenza. More particularly, aspects of the present invention relate to a multivalent use of these DNA constructs, to offer a wider umbrella of protection against infection by influenza. Novel biological tools, prophylactics, therapeutics, diagnostics, and methods of use of the foregoing are embodiments.

### BACKGROUND OF THE INVENTION

Avian influenza is highly pathogenic and causes severe multi-organ disease in poultry, resulting in devastating socio-economic losses in various parts of the world. In addition to socio economic losses, the greatest threat posed by this virus, however, is its ability to cause lethal human infections with the capacity of becoming pandemic. To date the most likely source of lethal human avian influenza is most likely from poultry.

Various approaches have been used to combat the virus in its natural avian host, including inactivated viral vaccines and live attenuated vaccines, both of which are currently licensed for use in poultry. Subbarao K, et al. (2007) PLoS Pathog 3: e40; Subbarao K, et al. (2007) Nat Rev Immunol 7: 267-278; Webby R J, et al. (2003) Science 302: 1519-1522; Stohr K (2005) N Engl J Med 352: 405-407; Stohr K, et al. (2004) Science 306: 2195-2196. Additionally, live viral vectors that express influenza virus proteins (Qiao C L, et al. (2003) Avian Pathol 32: 25-32; Hoelscher M A, et al. (2006) Lancet 367: 475-481) and reverse genetic vaccines (Hatta M, et al. (2001) Science 293: 1840-1842) are in development. An attempt to induce a broad range immune response against the highly lethal 1918 virus, which caused an unprecedented pandemic in humans, using a DNA vaccine that encodes HA has been reported. Kong W-P, et al. (2006) Proc Natl Acad Sci USA 103: 15987-15991.

DNA vaccines have been shown to elicit a robust immune response in various animals including mice and nonhuman primates, and most importantly in human trials against various infectious agents including influenza, SARS, SIV and HIV. Barry M A, et al. (1997) Vaccine 15: 788-791; Robinson H L, et al. (1997) Semin Immunol 9: 271-283; Gurunathan S, et al. (2000) Annu Rev Immunol 18: 927-974; Kodihalli S, et al. (2000) Vaccine 18: 2592-2599; Yang Z-Y, et al. (2004) Nature 428: 561-564; Lee C W, et al. (2006) Clin Vaccine Immunol 13: 395-402; Gares S L, et al. (2006) Clin Vaccine Immunol 13: 958-965; Roh H J, et al. (2006) J Vet Sci 7: 361-368; Swayne D E (2006) Ann N Y Acad Sci 1081: 174-181; Kumar M, et al. (2007) Avian Dis 51: 481-483; Luckay A, et al. (2007) J Virol 81: 5257-5269. DNA vaccines not only generate robust antibody responses but can also elicit strong T cell responses. Barry M A, et al. (1997) Vaccine 15: 788-791; Robinson H L, et al. (1997) Semin Immunol 9: 271-283; Gurunathan S, et al. (2000) Annu Rev Immunol 18: 927-974; Gares S L, et al. (2006) Clin Vaccine Immunol 13: 958-965; McCluskie M J, et al. (1999) Mol Med 5: 287-300; Raviprakash K, et al. (2006) Methods Mol Med 127: 83-89. DNA vaccination has been used in a variety of mammals including cattle (Skinner M A, et al.\ (2003) Infect Immun 71: 4901-4907; Ruiz L M, et al. (2007) Vet Parasitol 144: 138-145), pigs (Selke M, et al. (2007) Infect Immun 75: 2476-2483), penguins (Sherrill J, et al. (2001) J Zoo Wildl Med 32: 17-24; Grim K C, et al. (2004) J Zoo Wildl Med 35: 154-161) and horses (Kutzler M A, et al. (2004) J Am Vet Med Assoc 225: 414-416). DNA vaccines have also been used in a number of birds including chickens (Lee C W, et al. (2006) Clin Vaccine Immunol 13: 395-402; Roh H J, et al. (2006) J Vet Sci 7: 361-368), ducks (Gares S L, et al. (2006) Clin Vaccine Immunol 13: 958-965) and turkeys (Gares S L, et al. (2006) Clin Vaccine Immunol 13: 958-965; Kapczynski D R, et al. (2003) Avian Dis 47: 1376-1383; Verminnen K, et al. (2005) Vaccine 23: 4509-4516). The use of DNA vaccines in the avian model has been extensively reviewed (Oshop G L, et al. (2002) Vet Immunol Immunopathol 89: 1-12).

The development and characterization of a DNA vaccine to immunize humans, horses, cats, dogs, pigs and other farm animals, especially poultry, has far-reaching implications in the fight against the H5N1 epidemic, as the virus may infect a wide range of animal populations. Webster R G, et al. (2002) J Virol 76: 118-126; Capua I, et al. (2007) Vaccine 25: 5645-5652; Sorrell E M, et al. (2007) Cytogenet Genome Res 117: 394-402. While there is marked diversity in the host infectability of type A influenza viruses, due to the great diversity among birds, it is widely believed that the possibility of a pandemic strain of type A influenza will likely arise from genes contributed by HPAI H5N1. Longini I M, Jr., et al. (2005) Science 309: 1083-1087. In spite of the effectiveness of the DIVA (differentiating infected from vaccinated animals) system based on heterologous vaccination (Suarez D L (2005) Biologicals 33: 221-226), conventional inactivated vaccination modalities may not fully prevent secondary outbreaks depending on the flocks' ecological and epidemiological dynamics and the vaccine strains' homology to the field strain (Capua I, et al. (2007) Vaccine 25: 5645-5652). Accordingly, the need for effective immunogens and vaccination schema that would provide a potent and broad immune response in all animal species that are susceptible to Avian Influenza infection is manifest. Lee C W, et al. (2006) Clin Vaccine Immunol 13: 395-402; Subbarao K, et al. (2007) PLoS Pathog 3: e40; Subbarao K, et al. (2007) Nat Rev Immunol 7: 267-278. There is a need for a multivalent DNA vaccine for poultry that can protect against multiple HPAI H5N1 strains with a wider homologous and heterologous umbrella of protection and that could keep pace with the continued evolution of avian influenza viruses. That is, there is a need for a vaccine that confers protection against challenge from a strain of HPAI H5N1, or other strains that is different than the strain from which the vaccine is derived.

There is also a need for multivalent DNA immunogens and vaccines for horses and pigs. Equine influenza, caused by Equine-1 (H7N7) or Equine-2 (H3N8) influenza virus, is endemic to horses. It exhibits a high rate of transmission, short incubation period, and often causes infection in nearly all (˜100%) exposed, unvaccinated horses. Outbreaks of equine influenza in the US, Europe, Japan and Australia have caused severe economic impacts. Similarly, swine influenza, caused by swine H1N1 or H3N2 influenza virus, is very contagious and is a global threat. Swine influenza can also infect turkeys and humans without further spread. The economic impact of swine influenza can also be severe since infected animals exhibit retarded weight gain, thereby taking longer to reaching market weight. Accordingly, the need for effective immunogens and vaccination schema that would provide potent and broad immune responses in all animals that are susceptible to equine influenza or swine influenza is clear.

In addition, currently available inactivated vaccines are grown in embryonated eggs, a process that requires large biocontainment facilities and can take several months to produce. This inefficient production model is highly disadvantageous because it also severely limits response times to new emerging virus strains. Thus, there is a need to produce vaccines that are not produced by egg-based technology, and which can be tailored to newly emerging strains. In addition, there is a need to manufacture vaccines at a faster rate to increase the response rate to frequently evolving viruses. There is also a need for vaccines that elicit immune responses with increased breadth and magnitude, including more robust T cell responses. There is also a need for vaccines that enable the ability to differentiate between infected and vaccinated animals.

### SUMMARY OF THE INVENTION

Some embodiments are directed to an influenza vaccine or immunogenic composition comprising one or more DNA constructs that encode at least two divergent HAs such as H5 HAs, wherein each of said one or more DNA constructs encode one or more of said at least two divergent HAs such as H5 HAs, wherein an immune response is induced to a plurality of strains of influenza virus such as H5 influenza virus upon administration of the vaccine to a subject, wherein at least one strain of the plurality of strains is not the same strain as each strain that contains a gene that encodes each of said at least two divergent HAs such as H5 HAs. In some aspects, the one or more DNA constructs encode at least three divergent H5 HAs. In other aspects, each of the one or more DNA constructs encode one of said at least two divergent HAs such as H5 HAs. In other aspects, immunogenicity due to administration of the influenza vaccine or an immunogenic composition is conferred for the lifespan of the subject. In one embodiment, one DNA construct of the least one DNA construct encodes H5 HA from A/Indonesia/05/2005. In other aspects, the one or more DNA constructs may encode HA from: a) A/Anhui/1/2005), A/Indonesia/05/2005, and A/chicken/Nigeria/641/2006; b) A/Indonesia/05/2005, A/Anhui/1/2005 and A/Vietnam/1203/2004; c) A/Hong Kong/156/1997, A/chicken/Korea/ES/2003, A/turkey/Turkey/1/2005, A/Egypt/2782-NAMRU3/2006, and A/chicken/Nigeria/641/2006; or d) A/Indonesia/05/2005, A/Anhui/1/2005, A/Thailand/1(KAN-1)/2004, A/Hong Kong/483/1997, and A/Iraq/207-NAMRU3/2006.

In other embodiments, the vaccine or immunogenic composition confers complete or partial protection in mice, ferrets or chickens against H5N1 A/Vietnam/1203/2004 after vaccination. In other embodiments, the vaccine or immunogenic composition confers complete or partial protection in pigs and horses against H3 influenza virus such as H3N1.

Other embodiments include a method of inducing an immune response to a plurality of strains of influenza virus such as H5 influenza virus in a subject, such as a bird or chicken, in need thereof, comprising: optionally identifying a subject or bird in need of an immune response against various strains of influenza virus such as H5 influenza virus; and administering one or more DNA constructs that encode at least two divergent HAs such as H5 HAs to said subject, wherein each of said one or more DNA constructs encode one or more of said at least two divergent HAs such as H5 HAs, wherein an immune response is induced to the plurality of strains of influenza virus such as H5 influenza virus upon administration of the vaccine to a subject, wherein at least one strain of the plurality of strains is not the same strain as each strain that contains a gene that encodes each of said two divergent HAs such as H5 HAs. The one or more DNA constructs may be delivered to the dermis and/or subcutaneous tissue of the subject or bird.

Other embodiments include a use of one or more DNA constructs encoding at least two divergent HAs such as H5 HAs, wherein each of said one or more DNA constructs encode one or more of said divergent HAs such as H5 HAs, to elicit an immune response in a bird to a plurality of strains of influenza virus such as H5 influenza virus upon administration to the bird, wherein at least one strain of the plurality of strains is not the same strain as each strain that contains a gene that encodes each of said two divergent HAs such as H5 HAs, wherein said one or more DNA constructs are formulated in a composition for dermal and subcutaneous delivery. Other embodiments include a use of one or more DNA constructs encoding at least two divergent HAs such as H5 HAs, wherein each of said one or more DNA constructs encode one or more of said divergent HAs such as H5 HAs, in the preparation of a medicament that induces an immune response in a bird to a plurality of strains of influenza virus such as H5 influenza virus, wherein at least one strain of the plurality of strains is not the same strain as each strain that contains a gene that encodes each of said two divergent HAs such as H5 HAs, wherein said one or more DNA constructs are formulated for dermal and/or subcutaneous delivery.

In one aspect of the embodiments described herein, the subject is selected from the group consisting of human, horse, bird, cat, dog, pig and other farm animals. In one aspect of the embodiments, the one or more DNA constructs are administered by a route selected from the group consisting of topical, intranasal, intraocular, subcutaneous, intramuscular, transdermal, intradermal, parenteral, gastrointestinal, transbronchial, and transalveolar. In one aspect, the DNA construct is administered by a needle-free injector, such as an Agro-Jet needle-free injector at a pressure of 45-52 p.s.i. In one aspect, the one or more DNA constructs encode at least three divergent H5 HAs. In some aspects, the requisite immune response is defined by the presence of neutralizing antibodies for at least four, for example, five divergent strains of H5 influenza virus. In some aspects of the embodiment, the one or more DNA constructs are administered in a formulation comprising an adjuvant. This adjuvant can be selected from the group consisting of cationic lipid, cationic liposome, immune stimulatory gene, and immune stimulatory cytokine. In some aspects, the divergent HAs are selected from H1, H2, H3, H4, H5, H6, H7, H8, H9, H10, H11, H12, H13, H14, and H15. In other aspects, the HAs are H5 HAs selected from the group consisting of SEQ ID NOs: 1, 2, 3, 4, 5, 6, 7, 8, 9, 10, and 11. In some embodiments, the one or more DNA constructs are codon-optimized for expression in humans. In other embodiments, the immune response is the presence of neutralizing antibodies for at least three, four or five divergent strains of influenza virus such as H5 influenza virus.

Another embodiment includes a method of preparing an immunogen capable of eliciting a response against at least three subtypes of a distinct strain of Influenza, comprising incorporating nucleic acids encoding at least two distinct HA peptides from the same strain into at least one expression vector to form one or more DNA constructs, wherein each of said one or more DNA constructs encode one or more of said distinct HA peptides, administering the one or more DNA constructs to an animal and determining whether the immunogen elicits an immune response in the animal to confer protection against at least three distinct peptides from the same strain. In one aspect, the strain of Influenza is selected from the group consisting of H1, H2, H3, H4, H6, H7, H8, H9, H10, H11, H12, H13, H14, and H15.

Another embodiment includes a method of identifying relevant vaccine components capable of eliciting a response against at least three subtypes of a divergent strain of Influenza for use in a recombinant-protein based immunogen, comprising incorporating nucleic acids encoding at least two distinct HA peptides from the same strain into at least one expression vector to form one or more DNA constructs, wherein each of said one or more DNA constructs encode one or more of said distinct HA peptides, administering said one or more DNA constructs to an animal, determining whether the immunogen elicits an immune response in the animal to confer protection against at least three distinct peptides from the same strain, and selecting the at least two subtypes capable of eliciting an immune response in the animal to confer protection against at least three distinct peptides for formulation as a recombinant-protein based immunogen. In one aspect, the strain of Influenza is selected from the group consisting of H1, H2, H3, H4, H6, H7, H8, H9, H10, H11, H12, H13, H14, and H15.

Other embodiments include a method of making the influence a vaccine in cells using conventional methods of making DNA constructs.

Some embodiments are directed to an influenza immunogen or influenza vaccine comprising one or more DNA constructs that encode at least two divergent influenza HAs, such as influenza H1 HAs, influenza H3 HAs, or a mixture of H1 and H3 influenza HAs. Each of such one or more DNA constructs encodes one or more of the at least two divergent influenza HAs. Such an influenza immunogen or influenza vaccine induces an immune response to a plurality of strains of influenza virus, such as H1 influenza virus, H3 influenza virus or a mixture of H1 and H3 influenza virus, upon administration of the immunogen or vaccine to a subject, wherein at least one strain of the plurality of strains does not encode any of the divergent influenza HAs (i.e., is not the same strain as each strain that contains a gene that encodes each of said at least two divergent HAs). In certain embodiments, the one or more DNA constructs encode at least two divergent influenza HAs, which are influenza H1 HAs, influenza H3 HAs, or a mixture of H1 and H3 influenza HAs. In certain embodiments, the one or more DNA constructs encode at least three divergent influenza HAs, which are influenza H1 HAs, influenza H3 HAs, or a mixture of H1 and H3 influenza HAs. In certain embodiments, each of the one or more DNA constructs encodes one or more of the at least two divergent influenza HAs, which are influenza H1 HAs, influenza H3 HAs, or a mixture of H1 and H3 influenza HAs. In certain embodiments, each of the one or more DNA constructs encodes one or more of at least three of the divergent influenza HAs, which are H1 HAs, influenza H3 HAs, or a mixture of H1 and H3 influenza HAs. In some aspects, immunogenicity due to administration of the influenza immunogen or vaccine is conferred for the lifespan of the subject. In certain embodiments, at least one of the one or more DNA constructs encodes H3 HA from Influenza A/equine/Bari/2005, Influenza A/equine/Ohio/1/2003, or Influenza A/equine/Aboyne/1/2005. In certain embodiments, one or more DNA constructs encode H3 HA from Influenza A/equine/Bari/2005, Influenza A/equine/Ohio/1/2003, and Influenza A/equine/Aboyne/1/2005. In certain embodiments, at least one of the one or more DNA constructs encodes H1 HA from Influenza A/swine/California/04/2009 or Influenza A/swine/Ohio/51145/2007. In certain embodiments, at least one of said one or more DNA constructs encodes H3 HA from Influenza A/swine/North Carolina/R08-001877/2008. In certain embodiments, one or more of the DNA constructs encode H1 HA from Influenza A/swine/California/04/2009, H1 HA from Influenza A/swine/Ohio/51145/2007, and H3 HA from Influenza A/swine/North Carolina/R08-001877/2008. In certain embodiments, at least one of the DNA constructs encodes an influenza HA having at least one of the following amino acid sequences: SEQ ID NO:30, SEQ ID NO:32, SEQ ID NO:34, SEQ ID NO:36, SEQ ID NO:38, or SEQ ID NO:40. In certain embodiments, at least one of the DNA constructs comprises CMV/R-Influenza A/swine/Ohio/51145/2007 (H1N1) HA/h (SEQ ID NO:29), CMV/R-Influenza A/swine/North Carolina/R08-001877/2008 (H3N2) HA/h (SEQ ID NO:31), CMV/R-Influenza A/swine/California/04/2009 (H1N1) HA/h (SEQ ID NO:33), CMV/R-Influenza A/equine/Ohio/1/2003 (H3N8) HA/h (SEQ ID NO:35), CMV/R-Influenza A/equine/Bari/2005 (H3N8) HA/h (SEQ ID NO:37), or CMV/R-Influenza A/equine/Aboyne/1/2005 (H3N8) HA/h (SEQ ID NO:39). In certain embodiments, at least one strain of the plurality of strains comprises an influenza virus having an H1 HA or an H3 HA. In certain embodiments, at least one of the DNA constructs encodes an influenza HA having at least one of the following amino acid sequences: SEQ ID NO:41, SEQ ID NO:42, SEQ ID NO:43, SEQ ID NO:44, SEQ ID NO:45, or SEQ ID NO:46.

In certain embodiments, the immunogen or vaccine, after administration, confers complete or partial protection in horses against an H3 influenza virus, such as H3H8 influenza virus. In certain embodiments, the immunogen or vaccine, after administration, confers complete or partial protection in pigs against H1 influenza virus, such as H1N1 influenza virus. In certain embodiments, the immunogen or vaccine, after administration, confers complete or partial protection in pigs against H3 influenza virus, such as H3N2 influenza virus. In certain embodiments, the immunogen or vaccine, after administration, confers complete or partial protection in pigs against H1 and H3 influenza virus, such as H1N1 influenza virus and H3N2 influenza virus.

In some embodiments, the one or more DNA constructs are codon-optimized for expression in humans. In some embodiments, the one or more DNA constructs are codon-optimized for expression in other mammals, such as horses, pigs, cats, dogs, or other mammalian farm animals.

In some embodiments, the immune response is the presence of neutralizing antibodies for at least three, four or five divergent strains of influenza virus such as H1 or H3 influenza virus.

The disclosure provides a method of inducing an immune response to a plurality of strains of influenza virus in a subject in need thereof. Such a method comprises administering one or more DNA constructs that encode at least two divergent influenza HAs, such as influenza H1 HAs, influenza H3 HAs, or a mixture of H1 and H3 HAs. Each of such one or more DNA constructs encodes one or more of the at least two divergent influenza HAs. According to such a method, an immune response is induced to a plurality of strains of influenza virus, such as H1 influenza virus, H3 influenza virus, or a mixture of H1 and H3 influenza virus, upon such administration, wherein at least one strain of the plurality of strains does not encode any of the divergent influenza HAs. In certain embodiments, the one or more DNA constructs encode at least two divergent influenza HAs, which are influenza H1 HAs, influenza H3 HAs, or a mixture of H1 and H3 influenza HAs. In certain embodiments, the one or more DNA constructs encode at least three divergent influenza HAs, which are influenza H1 HAs, influenza H3 HAs, or a mixture of H1 and H3 influenza HAs. In certain embodiments, each of the one or more DNA constructs encodes at least one of the at least two divergent influenza HAs, which are influenza H1 HAs, influenza H3 HAs, or a mixture of H1 and H3 influenza HAs. In certain embodiments, each of the one or more DNA constructs encodes at least one of at least three of the divergent influenza HAs, which are H1 HAs, influenza H3 HAs, or a mixture of H1 and H3 influenza HAs. In some aspects, immunogenicity due to administration of the one or more DNA constructs is conferred for the lifespan of the subject. In certain embodiments, at least one of the one or more DNA constructs encodes H3 HA from Influenza A/equine/Bari/2005, Influenza A/equine/Ohio/1/2003, or Influenza A/equine/Aboyne/1/2005. In certain embodiments, one or more of the one or more DNA constructs encodes H3 HA from Influenza A/equine/Bari/2005, Influenza A/equine/Ohio/1/2003, and Influenza A/equine/Aboyne/1/2005. In certain embodiments, at least one of the one or more DNA constructs encodes H1 HA from Influenza A/swine/California/04/2009 or Influenza A/swine/Ohio/51145/2007. In certain embodiments, at least one of the one or more DNA constructs encodes H3 HA from Influenza A/swine/North Carolina/R08-001877/2008. In certain embodiments, one or more of the DNA constructs encodes H1 HA from Influenza A/swine/California/04/2009, H1 HA from Influenza A/swine/Ohio/51145/2007 and H3 HA from Influenza A/swine/North Carolina/R08-001877/2008. In certain embodiments, at least one of the DNA constructs encodes an influenza HA having an amino acid sequence selected from the group consisting of SEQ ID NO:30, SEQ ID NO:32, SEQ ID NO:34, SEQ ID NO:36, SEQ ID NO:38, and SEQ ID NO:40. In certain embodiments, at least one of the DNA constructs comprises SEQ ID NO:29, SEQ ID NO:31, SEQ ID NO:33, SEQ ID NO:35, SEQ ID NO:37, or SEQ ID NO:39. In certain embodiments, at least one of the DNA constructs comprises SEQ ID NO:41, SEQ ID NO:42, SEQ ID NO:43, SEQ ID NO:44, SEQ ID NO:45, or SEQ ID NO:46. In certain embodiments, at least one strain of the plurality of strains comprises an influenza virus having an H1 HA or an H3 HA. In certain embodiments, the influenza virus is an equine influenza virus or a swine influenza virus.

In certain embodiments of such a method to induce an immune response, the subject is a human, horse, pig, bird, such as a turkey, cat, dog, or other farm animal. In certain embodiments, the one or more DNA constructs are administered by a topical, intranasal, intraocular, subcutaneous, transdermal, intradermal, intramuscular, parenteral, gastrointestinal, transbronchial, or transalveolar route. In certain embodiments, the route is intramuscular. In certain embodiments, the DNA constructs are administered by a needle-free injector, such as an AGRO-JET® needle-free injector or a PHARMAJET® SC/IM Injection System. In certain embodiments, the one or more DNA constructs are administered in a formulation comprising an adjuvant. In certain embodiments, such a method confers complete or partial protection in horses against an H3 influenza virus, such as H3H8 influenza virus. In certain embodiments, such a method confers complete or partial protection in pigs against H1 influenza virus, such as H1N1 influenza virus. In certain embodiments, such a method confers complete or partial protection in pigs against H3 influenza virus, such as H3N2 influenza virus. In certain embodiments, such a method confers complete or partial protection in pigs against H1 and H3 influenza virus, such as H1N1 influenza virus and H3N2 influenza virus. In some embodiments, the one or more DNA constructs are codon-optimized for expression in humans. In some embodiments, the one or more DNA constructs are codon-optimized for expression in other mammals, such as horses, pigs, cats, dogs, or other mammalian farm animals. In some embodiments, the immune response is the presence of neutralizing antibodies for at least three, four or five divergent strains of influenza virus such as H1 or H3 influenza virus.

The disclosure provides an influenza HA protein comprising at least one of the following amino acid sequences: SEQ ID NO:30, SEQ ID NO:32, SEQ ID NO:34, SEQ ID NO:36, SEQ ID NO:38, or SEQ ID NO:40. The disclosure also includes a DNA construct comprising a nucleic acid molecule encoding one or more of such an influenza HA protein. Such a DNA construct can be a vector that expresses such a nucleic acid molecule, such as a CMV vector, such as a CMV/R vector.

The disclosure also provides a DNA construct encoding an HA protein comprising at least one of the following amino acid sequences: SEQ ID NO:30, SEQ ID NO:32, SEQ ID NO:34, SEQ ID NO:36, SEQ ID NO:38, or SEQ ID NO:40. The disclosure also provides a DNA construct comprising a nucleic acid molecule encoding one or more of such an influenza HA protein. Such a DNA construct can be a vector that expresses such a nucleic acid molecule, such as a CMV vector, such as a CMV/R vector.

The disclosure also provides a DNA construct comprising at least one of the following nucleic acid sequences: SEQ ID NO:41, SEQ ID NO:42, SEQ ID NO:43, SEQ ID NO:44, SEQ ID NO:45, SEQ ID NO:46, SEQ ID NO:29, SEQ ID NO:31, SEQ ID NO:33, SEQ ID NO:35, SEQ ID NO:37, and SEQ ID NO:39. The disclosure also provides a DNA construct comprising at least one of the following nucleic acid sequences: SEQ ID NO:29, SEQ ID NO:31, SEQ ID NO:33, SEQ ID NO:35, SEQ ID NO:37, and SEQ ID NO:39. The disclosure also provides a DNA construct comprising at least one of the following nucleic acid sequences: SEQ ID NO:41, SEQ ID NO:42, SEQ ID NO:43, SEQ ID NO:44, SEQ ID NO:45, SEQ ID NO:46. Such a DNA construct can be a vector that expresses such a nucleic acid molecule, such as a CMV vector, such as a CMV/R vector.

Also included are methods to prepare such immunogens, vaccines and DNA constructs.

### BRIEF DESCRIPTION OF THE DRAWINGS

FIG. 1A-C are graphs depicting the ability of sera derived from mice immunized with various multivalent H5 HA Influenza DNA immunogens to neutralize broadly divergent Influenza strains. The breadth of neutralization against divergent HAs is determined by the composition of multivalent HA DNA constructs. Neutralization of 12 different H5N1 isolates was determined in the LAI assay using serum from mice immunized with (A) 10 HAs comprised of: pCMV/R 8κB-HA(A/Hong Kong/156/1997), pCMV/R 8κB-HA(A/chicken/Korea/ES/2003), pCMV/R-HA(A/turkey/Turkey/1/2005), pCMV/R-HA(A/Egypt/2782-NAMRU3/2006), pCMV/R-HA(A/chicken/Nigeria/641/2006), pCMV/R 8κB-HA(A/Indonesia/05/2005), pCMV/R-HA(A/Anhui/1/2005), pCMV/R 8κB-HA(A/Thailand/1(KAN-1)/2004), pCMV/R 8κB-HA(A/Hong Kong/483/1997), and pCMV/R-HA(A/Iraq/207-NAMRU3/2006) as in 5 HA (Set 1) plus in 5 HA (Set 2); (B) 5 HA (Set 1) composed of DNA constructs: pCMV/R 8κB-HA(A/Hong Kong/156/1997), pCMV/R 8κB-HA(A/chicken/Korea/ES/2003), pCMV/R-HA(A/turkey/Turkey/1/2005), pCMV/R-HA(A/Egypt/2782-NAMRU3/2006), and pCMV/R-HA(A/chicken/Nigeria/641/2006); or (C) 5 HA (Set 2) contained: pCMV/R 8κB-HA(A/Indonesia/05/2005), pCMV/RB-HA(A/Anhui/1/2005), pCMV/R 8κB-HA(A/Thailand/1(KAN-1)/2004), pCMV/R 8κB-HA(A/Hong Kong/483/1997), and pCMV/R-HA(A/Iraq/207-NAMRU3/2006). Mice were vaccinated as described herein. In this experiment, the DNA vaccine consisted of 10 DNA constructs (1.5 μg each) expressing HA proteins as indicated. In panels B and C, mice (n=10) were immunized with 15 μg of DNA construct (3 μg each) three times at 3 week intervals. Serum pools from the immunized animals were collected 14 days after the third immunization. The antisera were tested against the 12 indicated pseudotyped lentiviral vectors at varying dilutions. Error bars at each point indicate the standard deviation; each sample was evaluated in triplicate. In general, the immunized serum neutralized all tested pseudotyped lentiviruses at low dilutions while differences were often observed at high dilution.

FIG. 2 depicts the AGRO-JET® needle-free injector.

FIGS. 3A-L depict DNA constructs containing DNA coding for various H5 HA peptides.

FIG. 4 depicts the scheme for immunizing mice with various H5 HA DNA immunogens and collecting sera from the mice.

FIG. 5 shows graphs depicting the degree of neutralization displayed by mice immunized with individual H5 HA DNA against various homologous and heterologous HA pseudotyped lentiviral vectors. Sera from mice immunized with DNA vaccines encoding HA from specific strains neutralize a homologous and a heterologous HA with differing efficacy. Groups of mice (n=10) were immunized as described herein with 15 μg of individual H5 HA DNA construct, pCMV/R or pCMV/R 8κB, encoding the HA of indicated viruses: A/Indonesia/05/2005, A/Thailand/1(KAN-1)/2004, A/Hong Kong/156/1997, A/Hong Kong/483/1997, A/chicken/Korea/ES/2003, and pCMV/R encoding the HA of indicated viruses: A/Anhui/1/2005, A/turkey/Turkey/1/2005, A/Egypt/2782-NAMRU3/2006, A/chicken/Nigeria/641/2006, and A/Iraq/207-NAMRU3/2006. Sera were collected from each group 14 days after the third immunization, pooled, and tested against the homologous (open circles) or a heterologous HA, A/Vietnam/1203/2004 (black diamonds). Serum from each group was serially diluted (1:100 to 1:6400) and analyzed by LAI. Error bars at each point indicate the standard deviation; each sample was evaluated in triplicate. Different degrees of neutralization among various H5 pseudoviruses were observed among different HA-immunized mice.

FIG. 6 depicts an Experimental timeline indicating the immunization schedule, sera sample collection, and challenge dates in mice.

FIG. 7A depicts a leg-skin histology section after delivery of the vaccine via AGRO-JET® at 48 psi for intra-dermal/sub-cutaneous delivery.

FIG. 7B depicts the location of DNA vaccine delivery after administration with AGRO-JET® at various pressures.

FIG. 8A-C show graphs depicting immune protection after DNA vaccination against lethal challenge with heterologous A/Vietnam/1203/2004 using needle or needle-free injection in chickens and dose-response analysis of efficacy for each route.

A. Chickens were immunized with either trivalent HA encoding DNA constructs: pCMV/R-HA(A/Indonesia/05/2005) plus pCMV/R-HA(A/Anhui/1/2005) plus pCMV/R-HA(A/Vietnam/1203/2004) by Agro-Jet® three times, monovalent DNA construct, pCMV/R-HA(A/Indonesia/05/2005) with Agro-Jet® twice or three times, or by needle and syringe subcutaneously or IM three times as indicated. Control animals were injected with a no insert vector by needle and syringe IM three times. A total of 500 μg DNA construct was used in each injection for all groups. Controls died 2 days after infection by nasal inoculation. The geometric mean reciprocal endpoint titers (GMT) for hemagglutination inhibition (HI) in each group one week after the last vaccination and two weeks post-challenge respectively were: Control: undetectable, not done; Trivalent by Agro-Jet (×3): 777, 304; A/Indonesia/05/2005 by Agro-Jet (×3): 320, 285; A/Indonesia/05/2005 by Agro-Jet (×2): 516, 533; A/Indonesia/05/2005 by needle (subcutaneous ×3): 211, 155; A/Indonesia/05/2005 by Agro-Jet (intramuscular ×3): 118, 495.

B. Immunization with trivalent DNA vaccine, pCMV/R-HA(A/Anhui/1/2005), pCMV/R-HA(A/Indonesia/05/2005), and pCMV/R-HA(A/chicken/Nigeria/641/2006), twice by AGRO-JET® intradermally/subcutaneously in different doses (500 μg, 50 μg, 5 μg, and 0.5 μg) as indicated. Controls were immunized with 500 μg empty vector. Controls died 4 days after infection by intranasal inoculation. The GMT HI in each group two weeks post-challenge were: Control: 80 (n=1); Trivalent by Agro-Jet (500 μg): 580 (n=8); Trivalent by Agro-Jet (50 μg): 430 (n=8); Trivalent by Agro-Jet (5 μg): 183 (n=8); Trivalent by Agro-Jet (0.5 ng): 200 (n=2).

C. Immunization with trivalent DNA vaccine pCMV/R-HA(A/Anhui/1/2005), pCMV/R-HA(A/Indonesia/05/2005), and HA(A/chicken/Nigeria/641/2006) was performed twice by needle and syringe IM at doses outlined in panel B. The GMT HI in each group two weeks post-challenge were: Control: not done (n=0); Trivalent by needle (500 μg): 325 (n=8); Trivalent by needle (50 μg): 120 (n=8); Trivalent by needle (5 μg): 197 (n=8); Trivalent by needle (0.5 μg): 200 (n=2). The chickens in panel B and C each received two immunizations.

FIG. 9 depicts histologic analyses of tissues that received injections of India ink at various pressures. To evaluate the distribution of fluid into superficial or deep layers of subcutaneous tissues after delivery by AGRO-JET®, 4 or 7 week old chickens were injected with a solution containing India ink with this needle-free device at various pressures, ranging from 45 to 55 mm Hg. Three sites (thigh, wing and breast) were used, and biopsies were taken for routine hematoxylin and eosin staining Representative sections of thigh injections are shown from 7-week old chickens and were similar at 4 weeks. While the 48 mm Hg pressure deposited the injectate into the dermis/subcutaneous region (left), the higher pressure injections, 52 and 58 mm Hg, deposited the injectate into the subcutaneous and muscle layers (middle, right). 48 mm Hg consistently provided an optimal pressure to deposit the injectate into the dermis and subcutaneous tissue and was chosen for all AgroJet® immunizations.

FIG. 10 shows graphs depicting neutralizing antibody responses against homologous and heterologous HAs from chickens immunized with HA DNA construct by different routes. Neutralization against the indicated strain HAs was analyzed after immunization with trivalent HA encoding DNA constructs: pCMV/R-HA(A/Indonesia/05/2005), pCMV/R-HA(A/Anhui/1/2005), and pCMV/R-HA(A/Vietnam/1203/2004) with the indicated delivery device using sera taken two weeks after the third injection. Neutralization was determined by lentiviral assay inhibition assay (LAI) from individual chickens at titers ranging from 1:100 to 1:3200. The recombinant lentiviral vectors expressing a luciferase reporter gene were produced as previously described (Yang Z-Y, et al. (2007) Science 317: 825-828; Kong W—P, et al. (2006) Proc Natl Acad Sci USA 103: 15987-15991.) For the neutralization assay, antisera from immunized animals were heat-inactivated at 55° C. for 30 minutes and mixed with 50 μl of pseudovirus at various dilutions. The sera/virus mixture was then added to 293A cells in 96-well B&W TC Isoplates (Wallac, Turku, Finland; 12,000 cells/well). Two hours later, the plates were washed and fresh medium was added. Cells were lysed in mammalian cell lysis buffer (Promega, Madison, Wis.) 24 hrs. after infection and luciferase activity was measured using the Luciferase Assay System (Promega, Madison, Wis.). Bird #238 consistently showed a low level of neutralization, possibly because of an inhibitor in the serum because it was fully protected against viral challenge. Percent neutralization was calculated by the reduction of luciferase activity relative to the values achieved in the non-immune sera.

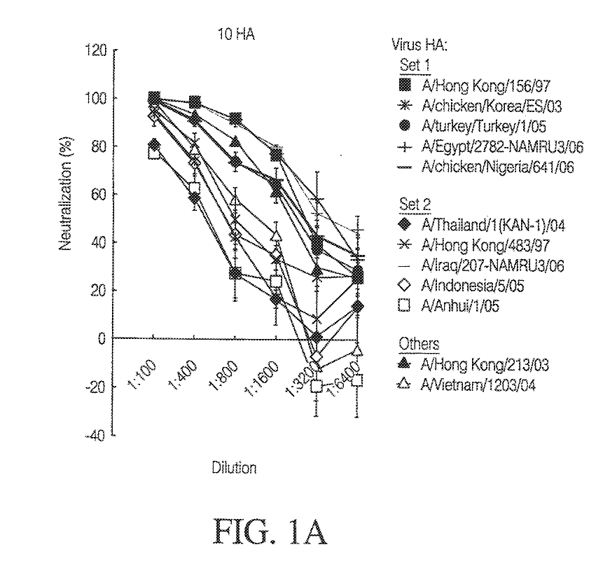

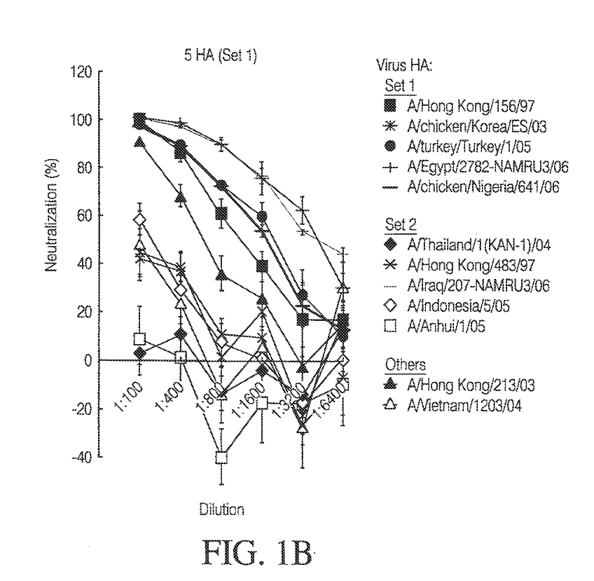

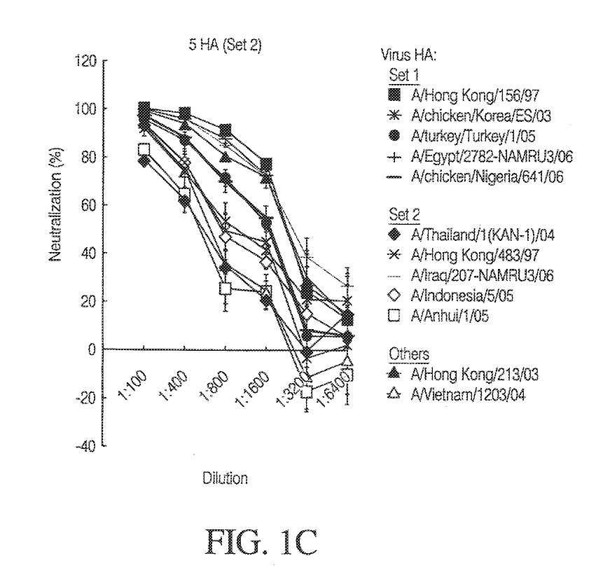

FIG. 11 shows immune protection conferred against lethal challenge of A/Vietnam/1203/2004 in mice 68 weeks after vaccination. Mice were immunized with 15 μg total of either the 10 HA as in FIG. 1A, legend and as described herein, 5 HA (Set 1) as in FIG. 1B, legend and as described herein, 5 HA (Set 2) as mentioned in FIG. 1C, legend and described herein, monovalent A/Indonesia/05/2005 HA, monovalent A/Anhui/1/2005 HA, or Control (empty vector) three times at three week intervals as described in FIG. 1, legend. Animals (n=8-10 per group) were challenged 68 weeks later by intranasal inoculation. All control mice died 10 days after infection.

FIG. 12 depicts maps for vectors and DNA constructs used in the present invention. (A) map of VCR8400 (CMV/R; SEQ ID NO:41); (B) map of VRC9336 (CNV/R-Influenza A/swine/Ohio/51145/2007 (H1N1) Ha/h; SEQ ID NO:29); (C) map of VRC9334 (CMV/R-Influenza A/swine/North Carolina/R08-001877/2008 (H3N2) Ha/h; SEQ ID NO:31); (D) map of VRC9328 (CMV/R-Influenza A/swine/California/04/09 (H1N1) Ha/h; SEQ ID NO:33); (E) map of VRC9254 (CMV/R-Influenza A/equine/Ohio/1/2003 (H3N8) HA wt; SEQ ID NO:35); (F) map of VRC9253 (CMV/R-Influenza A/equine/Bari/2005 (H3N8) HA wt; SEQ ID NO:37); (G) map of VRC9294 (CMV/R-Influenza A/equine/Aboyne/1/05 (H3N8) Ha/h; SEQ ID NO:39)

FIG. 13 depicts the survival time of chickens vaccinated with a trivalent vaccine comprising the DNA constructs CMV/R Influenza A/Indonesia/05/05 (H5N1) HA-mutA, CMV/R Influenza A/Nigeria/641/2006/(H5N1) HA-mutA, and CMV/R Influenza A/Anhui/1/2005/(H5N1) HA-mutA, with or without adjuvant, and challenged with H5N1 virus.

FIG. 14 depicts the average titer of virus shed from chickens vaccinated with a trivalent vaccine comprising the DNA constructs CMV/R Influenza A/Indonesia/05/05 (H5N1) HA-mutA, CMV/R Influenza A/Nigeria/641/2006/(H5N1) HA-mutA, and CMV/R Influenza A/Anhui/1/2005/(H5N1) HA-mutA, and challenged with H5N1. (A) shows the average titer of virus in the trachea. (B) shows the average titer of virus in the cloaca.

FIG. 15 depicts the antibody titer in horses vaccinated intramuscularly, using a needle and syringe (N&S), with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37), and then challenged with H3N8—Ohio/03 influenza virus. The graph shows the antibody titer measured against each of the Ohio/03, Richmond/07 and Aboyne/05 strains of influenza virus during the period of immunization and post-challenge. The titers depicted in the graph were determined using a hemagglutination inhibition assay.

FIG. 16 depicts the neutralizing antibody titer in horses vaccinated intramuscularly, using a needle and syringe (N&S), with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37), and then challenged with H3N8—Ohio/03 influenza virus. The graph shows the antibody titer against each of the Ohio/03, Richmond/07 and Aboyne/05 strains of influenza virus during the period of immunization and post-challenge.

FIG. 17 depicts post-challenge, rectal temperatures from horses vaccinated with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (N&S) or needle-free device (NF) and then challenged with H3N8—Ohio/03 influenza virus.

FIG. 18 depicts the antibody titer against (A) influenza A H3N8 strain A/eq/Richmond/1/07, (B) influenza A H3N8 strain A/equine/Ohio/03, and (C) influenza A H3N8 strain A/equine/Aboyne/05, in horses vaccinated with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37). The horses were vaccinated using either a needle and syringe (N&S), or needle-free device (NF), and then challenged with H3N8—Ohio/03 influenza virus. The titers depicted in the graph were determine using a single radial hemolysis assay (SRH) described by the OIE Manual of Diagnostic Tests and Vaccines for Terrestrial Animals¹, and Wood et al., 1983².

FIG. 19 depicts the antibody titer against (A) influenza A H3N8 strain A/eq/Ohio/03, and (B) influenza A H3N8 strain A/eq/Richmond/1/07, in horses vaccinated with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (N&S) or needle-free device (NF), and then challenged with H3N8—Ohio/03 influenza virus. The titers depicted in the graph were determined using a hemagglutination inhibition assay.

FIG. 20 depicts the viral load present in nasal secretions from horses vaccinated with sham vaccine (CMV/R; SEQ ID NO:41), monovalent vaccine comprising the DNA construct CMV/R-Influenza A/equine/Ohio/1/03 (H3N8) HA wt (SEQ ID NO:35), or trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (N&S) or needle-free device, and then challenged with H3N8—Ohio/03 influenza virus. The numbers listed in the chart represent copies of influenza virus RNA as determined using RT-PCR.

FIG. 21 depicts levels of interferon-γ mRNA in virus-stimulated peripheral blood mononuclear cells from horses vaccinated with sham vaccine (CMV/R; SEQ ID NO:41) or a trivalent vaccine comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using a needle-free device, and then challenged with H3N8—Ohio/03 influenza virus. The graph shows levels of IFN-mRNA before and after challenge, as determined using quantitative RT-PCR.

FIG. 22 depicts levels of (A) granzyme B mRNA and (B) interferon-γ mRNA in horses vaccinated with sham vaccine (CONTROL) (CMV/R; SEQ ID NO:41), monovalent vaccine (NF I) comprising the DNA construct CMV/R-Influenza A/equine/Ohio/1/03 (H3N8) HA wt (SEQ ID NO:35), or trivalent vaccine (NF II) comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (NS) or needle-free device (NF), and then challenged with H3N8—Ohio/03 influenza virus. Levels of cytokines were determined using quantitative RT-PCR.

FIG. 23 depicts levels of (A) interleukin-1 mRNA and (B) interleukin-6 mRNA in horses vaccinated with sham vaccine (CONTROL) (CMV/R; SEQ ID NO:41), monovalent vaccine (NF I) comprising the DNA construct CMV/R-Influenza A/equine/Ohio/1/03 (H3N8) HA wt (SEQ ID NO:35), or trivalent vaccine (NF II) comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (NS) or needle-free device (NF), and then challenged with H3N8—Ohio/03 influenza virus. Levels of cytokines were determined using quantitative RT-PCR.

FIG. 24 depicts levels of tumor necrosis factor alpha (TNF-α) in horses vaccinated with sham vaccine (CONTROL) (CMV/R; SEQ ID NO:41), monovalent vaccine (NF I) comprising the DNA construct CMV/R-Influenza A/equine/Ohio/1/03 (H3N8) HA wt (SEQ ID NO:35), or trivalent vaccine (NF II) comprising the DNA constructs CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using either a needle and syringe (NS) or needle-free device (NF), and then challenged with H3N8—Ohio/03 influenza virus. Levels of cytokines were determined using quantitative RT-PCR.

FIG. 25 depicts the titers of antibody to either Ohio/07, Cal-09 or Illinois/09 in pigs vaccinated with sham vaccine (CONTROL) (CMV/R; SEQ ID NO:41), monovalent vaccine (NF I) comprising the DNA construct CMV/R-Influenza A/swine/California/04/09 (H1N1) Ha/h (SEQ ID NO:33), or trivalent vaccine (NF II) comprising the DNA constructs CMV/R-Influenza A/swine/Ohio/51145/2007 (H1N1) Ha/h (SEQ ID NO:29), CMV/R-Influenza A/swine/North Carolina/R08-001877/2008 (H3N2) Ha/h (SEQ ID NO:31), and CMV/R-Influenza A/swine/California/04/09 (H1N1) Ha/h (SEQ ID NO:33), using either a needle and syringe (NS) or needle-free device (NF), and then challenged with either H1N1, H3N2 or influenza A/California/07/209 (H1N1). The titers depicted in the graph were determined using a hemagglutinin inhibition assay.

FIG. 26 depicts heterologous antibody titers against Ohio/07, Cal-09, or Illinois/09 in pigs vaccinated with sham vaccine, monovalent vaccine comprising the DNA construct CMV/R-Influenza A/swine/California/04/09 (H1N1) Ha/h (SEQ ID NO:33), or trivalent vaccine comprising the DNA constructs CMV/R-Influenza A/swine/Ohio/51145/2007 (H1N1) Ha/h (SEQ ID NO:29), CMV/R-Influenza A/swine/North Carolina/R08-001877/2008 (H3N2) Ha/h (SEQ ID NO:31), and CMV/R-Influenza A/swine/California/04/09 (H1N1) Ha/h (SEQ ID NO:33) using either a needle/syringe (NS) or needle-free device (NF). The titers depicted in the graph were determined using a hemagglutination inhibition assay after pigs received three immunizations.

FIG. 27 depicts the number of pigs testing positive for virus in nasal secretions following vaccination with sham vaccine (CMV/R; SEQ ID NO:41), monovalent vaccine comprising the DNA construct CMV/R-Influenza A/swine/California/04/09 (H1N1) Ha/h (SEQ ID NO:33), or trivalent vaccine comprising the DNA constructs CMV/R-Influenza A/swine/Ohio/51145/2007 (H1N1) Ha/h (SEQ ID NO:29), CMV/R-Influenza A/swine/North Carolina/R08-001877/2008 (H3N2) Ha/h (SEQ ID NO:31), and CMV/R-Influenza A/swine/California/04/09 (H1N1) Ha/h (SEQ ID NO:33) using either a needle and syringe (NS) or needle-free device (NF), and then challenged with either H1N1, H3N2 or influenza A/California/07/209 (H1N1).

### REFERENCES

1. OEI, Equine Influenza, OIE Manual of Diagnostic Tests and Vaccines for Terrestrial Animals. Paris, Office of International des Epizooties, 2004, pp. 686-697.
2. Wood, J. M., Mumford, J., Folkers, C. et al., Studies with inactivated equine influenza vaccine. 1. Serological responses of ponies to graded doses of vaccine. J. Hyg. (Load) 90:371-384, 1983.

### DETAILED DESCRIPTION OF THE INVENTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a DNA construct refers to one or more DNA constructs. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

The disclosure provides avian, equine and swine immunogens and vaccines as well as methods to produce and use such immunogens and vaccines.

Highly pathogenic avian influenza viruses have a global distribution and are very active in different parts of the world (Longini I M, Jr., et al. (2005) Science 309: 1083-1087; Webby R J, et al. (2003) Science 302: 1519-1522; Enserink M (2004) Science 306: 2016; Higginson R, et al. (2005) Br J Nurs 14: 632; Stohr K (2005) N Engl J Med 352: 405-407) and periodically cause endemic infections with devastating socioeconomic ‘downstream’ effects. More importantly, cross-species transmission to humans is possible and will lead to high rates of mortality. Longini I M, Jr., et al. (2005) Science 309: 1083-1087; Ferguson N M, et al. (2004) Science 304: 968-969; Neumann G, et al. (2006) Emerg Infect Dis 12: 881-886. While vaccines offer the potential to control avian disease, a major concern of current vaccines is their inability to protect against evolving avian influenza viruses. The pandemic threat of this virus, especially as it relates to the poultry industry and for reservoir avian hosts, increases the need for a vaccine that offers a broad spectrum immune response with near total protection against viral challenge by current and evolving avian influenza viruses. The only current limitation of the virus is its restricted ability to infect humans and undergo efficient human-to-human transmission. Longini I M, Jr., et al. (2005) Science 309: 1083-1087; Ungchusak K, et al. (2005) N Engl J Med 352: 333-340. Accordingly, a first line of defense against the disease could be mounted against the virus in its natural host: the avian species is contemplated. Defense against the virus in other hosts are also contemplated, e.g., equine species. Equine and swine influenza virus strains also have global distribution and can cause severe economic impact, as disclosed herein and as is known to those skilled in the art.

The invention provides an influenza immunogen or influenza vaccine comprising one or more DNA constructs that encode at least two divergent influenza HAs. Each of such one or more DNA constructs encodes one or more of the at least two divergent influenza HAs. Such an influenza immunogen or influenza vaccine induces an immune response to a plurality of strains of influenza virus upon administration of the immunogen or vaccine to a subject, wherein at least one strain of the plurality of strains does not encode any of the divergent influenza HAs. As such, the invention provides homologous and heterologous protection against influenza strains, thereby providing an umbrella of protection against current and evolving influenza virus. The invention also provides methods to use such one or more DNA constructs to induce an immune response to a plurality of strains of influenza virus wherein at least one strain of the plurality of strains does not encode any of the divergent influenza HAs.

DNA vaccines provide several advantages over protein-based vaccines, including the ability to express diverse antigens, tolerability in various hosts, and ease of delivery. DNA vaccination has also been shown to be safe and effective. Robinson H L, et al. (1997) Semin Immunol 9: 271-283; Kodihalli S, et al. (2000) Vaccine 18: 2592-2599; McCluskie M J, et al. (1999) Mol Med 5: 287-300; Oshop G L, et al. (2002) Vet Immunol Immunopathol 89: 1-12; Rao S S, et al. (2006) Vaccine 24: 367-373. DNA can be synthesized in a relatively short period of time, and the DNA constructs can be rapidly modified to target mutations that are specific for particular viral substrains. In this manner, a focused and enhanced immune response can be obtained. Gurunathan S, et al. (2000) Annu Rev Immunol 18: 927-974; Fomsgaard A (1999) Immunol Lett 65: 127-131; Wan H, et al. (2007) J Virol 81: 5181-5191. This may be particularly relevant in the event of an outbreak, wherein specificity is important for epidemic control.

The phrase “one or more DNA constructs that encode at least two divergent influenza HAs” includes embodiments in which each DNA construct encodes a single influenza HA; embodiments in which each DNA construct encodes more than one influenza HA; and embodiments in which one or more of the DNA constructs encodes a single influenza HA and while other DNA constructs encode more than one influenza HA. An immunogen of the disclosure can include one or more DNA constructs. For example, a trivalent immunogen comprising one or more constructs encoding three HAs can include, for example, three DNA constructs each of which encodes one of the three HAs; two constructs, one of which encodes one HA, and the other of which encodes two HAs; or one construct that encodes all three HAs.

The term “DNA construct” as used herein has its ordinary meaning as known to those skilled in the art and includes an artificially constructed DNA construct that contains a DNA insert, which contains a nucleic acid sequence encoding a protein of interest, e.g., HA. In some aspects, a DNA construct comprises a plasmid or an expression vector, such as pCMV/R or pCMV/R 8κB. In other aspects, a DNA construct may be delivered to a target tissue or cell. Examples of DNA constructs are: pCMV/R HA(A/Hong Kong/156/1997), pCMV/R HA(A/chicken/Korea/ES/2003), pCMV/R-HA(A/turkey/Turkey/1/2005), pCMV/R-HA(A/Egypt/2782-NAMRU3/2006), pCMV/R-HA(A/chicken/Nigeria/641/2006), pCMV/R HA(A/Indonesia/05/2005), pCMV/R-HA(A/Anhui/1/2005), pCMV/R HA(A/Thailand/1(KAN-1)/2004), pCMV/R HA(A/Hong Kong/483/1997), and pCMV/R-HA(A/Iraq/207-NAMRU3/2006), or their pCMV/R 8κB counterparts. Additional examples of DNA constructs are CMV/R-Influenza A/swine/Ohio/51145/2007 (H1N1) HA/h (SEQ ID NO:29), CMV/R-Influenza A/swine/North Carolina/R08-001877/2008 (H3N2) HA/h (SEQ ID NO:31), CMV/R-Influenza A/swine/California/04/2009 (H1N1) HA/h (SEQ ID NO:33), CMV/R-Influenza A/equine/Ohio/1/2003 (H3N8) HA/h (SEQ ID NO:35), CMV/R-Influenza A/equine/Bari/2005 (H3N8) HA/h (SEQ ID NO:37), or CMV/R-Influenza A/equine/Aboyne/1/2005 (H3N8) HA/h (SEQ ID NO:39).

The invention includes a DNA construct comprising at least one of the following nucleic acid sequences: SEQ ID NO:41, SEQ ID NO:42, SEQ ID NO:43, SEQ ID NO:44, SEQ ID NO:45, SEQ ID NO:46, SEQ ID NO:29, SEQ ID NO:31, SEQ ID NO:33, SEQ ID NO:35, SEQ ID NO:37, or SEQ ID NO:39. Certain embodiments include a DNA construct comprising at least one of the following nucleic acid sequences: SEQ ID NO:41, SEQ ID NO:42, SEQ ID NO:43, SEQ ID NO:44, SEQ ID NO:45, or SEQ ID NO:46. Certain embodiments include a DNA construct comprising at least one of the following nucleic acid sequences: SEQ ID NO:29, SEQ ID NO:31, SEQ ID NO:33, SEQ ID NO:35, SEQ ID NO:37, or SEQ ID NO:39. The invention also includes a DNA construct encoding at least one of the following amino acid sequences: SEQ ID NO:30, SEQ ID NO:32, SEQ ID NO:34, SEQ ID NO:36, SEQ ID NO:38, or SEQ ID NO:40.

As used herein, the term “vector” has its ordinary meaning as known to those skilled in the art and includes a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. In one embodiment, viral vectors (e.g., replication defective retroviruses or lentiviruses) serve equivalent functions.

As used herein, the term “HA” has its ordinary meaning as known to those skilled in the art and includes an antigenic subtype such as from H1, H2, H3, H4, H5, H6, H7, H8, H9, H10, H11, H12, H13, H14, and H15 of hemagglutinin (HA) of Influenza A viruses. As used herein, the term “H5 HA” has its ordinary meaning as known to those skilled in the art and includes an antigenic subtype (H5) of HA. Nucleic acids or genes encoding the H5 HA subtype are useful in embodiments of the present invention. H5 HA may be derived from A/Hong Kong/156/1997, A/chicken/Korea/ES/2003, A/turkey/Turkey/1/2005, A/Egypt/2782-NAMRU3/2006, A/chicken/Nigeria/641/2006, A/Indonesia/05/2005, A/Anhui/1/2005, A/Thailand/1(KAN-1)/2004, A/Hong Kong/483/1997, and A/Iraq/207-NAMRU3/2006. As used herein, the term “H1 HA” has its ordinary meaning as known to those skilled in the art and includes an antigenic subtype (H1) of HA. Nucleic acids or genes encoding the H1 HA subtype are useful in embodiments of the present invention. H1 HA may be derived from Influenza A/swine/California/04/2009, Influenza A/swine/Ohio/51145/2007, or other swine influenza virus having an H1 subtype, such as H1N1. As used herein, the term “H3 HA” has its ordinary meaning as known to those skilled in the art and includes an antigenic subtype (H3) of HA. Nucleic acids or genes encoding the H3 HA subtype are useful in embodiments of the present invention. H3 HA may be derived from Influenza A/swine/North Carolina/R08-001877/2008. H3 HA may be derived from Influenza A/swine/Illinois/2009 or other swine influenza virus having an H3 subtype, such as H3N2. H3 HA may be derived from Influenza A/equine/Bari/2005, Influenza A/equine/Ohio/1/2003, or Influenza A/equine/Aboyne/1/2005. H3 HA may be derived from Influenza/A/Richmond/1/2007, Influenza/A/Richmond/2/2007, or other equine influenza virus having an H3 subtype, such as H3N8.

In certain embodiments, an influenza HA is a swine influenza HA. A number of swine virus variants have been isolated, including those that are H1N1, H1N2, H1N7, H2N3, H3N1, H3N3, H3N8, H4N6, H5N1, and H9N2. DNA constructs encoding swine H1 HA, H2 HA, H3 HA, H4 HA, H5 HA or H9 HA subtypes are useful embodiments of the invention. The HA can be derived from, for example, Influenza A/swine/California/04/2009 (H1N1), Influenza A/swine/Ohio/51145/2007 (H1N1), Influenza A/swine/North Carolina/R08-001877/2008 (H3N2), Influenza A/swine/Illinois/2009 (H3N2), Influenza A/swine/Iowa/03032/2010 (H1N1), Influenza A/swine/Minnesota/03018/2010 (H1N2), Influenza A/swine/England/191973/92 (H1N7), Influenza A/swine/Missouri/4296424/2006 (H2N3), Influenza A/swine/Minnesota/63607-5/2008 (H3N1), Influenza A/swine/Ontario/42729A/01 (H3N3), Influenza A/swine/Anhui/01/2006 (H3N8), Influenza A/swine/Ontario/01911-1/99 (H4N6), Influenza A/swine/Gianyar-Indonesia/07/2006 (H5N1), or Influenza A/swine/Hebei/012/2008 (H9N2).

In certain embodiments, an influenza HA is an equine influenza HA. A number of equine virus variants have been isolated, including those that are H3N8, H5N1, and H7N7. DNA constructs encoding equine H3 HA, H5 HA or H7 HA subtypes are useful embodiments of the invention. An HA can be derived from, for example, Influenza A/equine/Bari/2005 (H3N8), Influenza A/equine/Ohio/1/2003 (H3N8), Influenza A/equine/Aboyne/1/2005 (H3N8), Influenza A/equine/Yokohama/aq19/2009 (H3N8), Influenza A/equine/Egypt/av1/2009 (H5N1), or Influenza A/equine/Newmarket/1/77 (H7N7).

The term “divergent HA” or “divergent H5 HA” or “divergent H1 HA” or “divergent H3 HA” includes HA encoded by a genetic variant or subtype of influenza that has a relatively unrelated phylogenetic relationship of an HA gene of different influenza A viruses. In one aspect the divergent HAs have the same serotype. At least two divergent HAs, such as H5 HAs, H1 HAs, or H3 HAs, includes, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 47, 48, 49, or 50 divergent HAs, such as H5 HAs, H1 HAs, or H3 HAs. Trees of representative influenza A viruses isolated in Asia were generated by the neighbor joining method in the PAUP* program and are discussed in Proc Natl Acad Sci USA. 2006 Nov. 7; 103(45): 16936-16941, which is incorporated herein by reference. The World Health Organization (WHO) phylogenetic trees are found on the WHO website on the world wide web at, for example: who.int/csr/disease/influenza/nomen.ppt; who.int/csr/disease/influenza/tree_large.pdf; and who.int/csr/disease/avian_influenza/smaltree.pdf. Phylogenetic trees for equine and swine influenza virus are also known to those skilled in the art.

Based on WHO classification of H5N1 viruses, the DNA construct based on the Indonesia insert described herein belongs to Glade 2.1.3, Nigeria is Glade 2.2, and Anhui is Glade 2.3.4. Inserts for the DNA constructs may be selected based on: phylogenetical analysis, the geographic isolation and the isolation time. Some isolates are phylogenetically very different, but they are old isolates and the chance of them to “resurface” may not be high. Thus, in addition to selection based on strains belonging to different subclades, other criteria may be used to select divergent HAs, such as H5 HAs, H1 HAs, or H3 HAs, such as selecting recent isolates (isolation time) and isolates that are found farther apart geographically (geographical isolation).

Examples of combinations of divergent HAs such as H5 HAs in the vaccines described herein based on the above criteria that are useful include: A) WhiteBackedMunia/HongKong/82820/07 (clade2.3.4), Egypt/0636NAMRU320/07 (Glade 2.2), and Indonesia/CDC1046/07 (Glade 2.1.3); B) Ck/Hunan/2292/06 (clade7), Indonesia/625/06 (Glade 2.1.2), and Nigeria/6e20/07 (Glade 2.2); and C) Ck/Nongkhai/NIAH400802/07 (Glade 2.3.4), Azerbaijan/001161/06 (Glade 2.2), and Indonesia/CDC1047/07 (Glade 2.1.3).

Other examples include: 1) A/chicken/Thailand/ICRC-V586/2008, A/chicken/Iran/53-3/2008, and A/whooper_swan/Hokkaido/2/2008; 2) A/brown-head_gull/Thailand/vsmu-4/2008, A/chicken/Kransnogvardeysk/58/2008, and A/grey_heron/Hong_Kong/1046/2008; and 3) A/chicken/Egypt/1709-6/2008, A/chicken/Phichi/NIAH600674/2008 and A/magpie_robin/Hong_Kong/1897/2008.

It is also expected that two different HAs selected on the basis of belonging to two subclades, diverse geographical regions, and/or having recent isolation times, will also provide protection or an immune response to a strain from an additional subclade. Examples include the following combinations: A) A/chicken/Thailand/ICRC-V586/2008 and A/whooper_swan/Hokkaido/2/2008; B) A/brown-head_gull/Thailand/vsmu-4/2008 and A/grey_heron/Hong_Kong/1046/2008; and C) A/chicken/Phichi/NIAH600674/2008 and A/magpie_robin/Hong_Kong/1897/2008.

However, three different HAs from three different subclades would be expected to have a greater breadth with respect to immune responses or protection, as three main subclades are circulating at this time. When more subclades are generated unexpectedly, as frequently is the case, three HAs from the three circulating subclades can cover at least most of the circulating subclades, in addition to providing an immune response or protection against an additional strain.

The WHO phylogenetic trees were generated based on, in part, the homology of HA between and among various strains of influenza. By amino acid sequence analysis, HAs from the trivalent DNA construct vaccine described in the examples (Indonesia, Anhui and Nigeria) are all in the range of 96% identical. This homology means these sequences have about 20 a.a. that are different from each other. In one embodiment, HAs, such as H5 HAs, H1 HAs, or H3 HAs, from divergent strains are 97% or less homologous between two strains, or any two strains in the vaccine. In other embodiments, the divergent strains are 96%, 95%, 94%, 93%, 92%, 91%, 90%, 89%, 88%, 87%, 86%, 85%, 84%, 83%, 82%, 81%, or 80%, or less homologous between two strains. In certain aspects, genes were chosen to express HAs, such as H5 HAs, H1 HAs, or H3 HAs, that were sufficiently different with respect to at least the phylogenetic analysis; that is, they are in different sub-clades. In some aspects, mice or other animals immunized with the viruses' HA gene alone elicit different degree of immune responses to other viruses, and can not cross react to non matching viruses well. Specifically, mice or other animals immunized with A viruses' HA can react well to A viruses, but may not react to viruses B that well. In some embodiments, with multi-HA in selected clades, broader coverage is expected in terms of immune responses and broader protection.

In certain aspects, divergent HAs, such as H5 HAs includes those HAs, such as H5 HAs that are sufficiently divergent such that an immune response may be induced to a plurality of strains of influenza virus, such as H5 influenza virus, wherein at least one strain of the plurality of strains is not the same strain of the least two divergent HAs such as H5 HAs. In certain aspects, the at least two divergent HAs, such as H1 HAs, H3 HAs, or a mixture thereof, includes HAs that are sufficiently divergent such that an immune response may be induced to a plurality of strains of influenza virus, such as H1 influenza virus, H3 influenza virus, or a mixture thereof, wherein at least one strain of the plurality of strains is not the same strain as that encoded by the at least two divergent HAs. Divergent HAs can include H1 HA, H2 HA, H3 HA, H4 HA, H5 HA or H9 HA swine subtypes and H3, H5 and H7 equine subtypes. An immune response can be induced against such H1 HA, H2 HA, H3 HA, H4 HA, H5 HA or H9 HA swine subtypes and H3, H5 and H7 equine subtypes. In certain embodiments, the immune response can protect against influenza virus comprising a different HA. For example an immunogen comprising one or more DNA constructs comprising a swine H3 HA may protect against infection by a swine H1N1 virus.

An “immune response” to an antigen or composition is the development in a subject of a humoral and/or a cellular immune response to an antigen present in the composition of interest. For purposes of embodiments of the present invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+T-cells. In addition, a chemokine response may be induced by various white blood or endothelial cells in response to an administered antigen.

Thus, an immunological response as used herein may be one that stimulates CTLs, and/or the production or activation of helper T-cells. The production of chemokines and/or cytokines may also be stimulated. The antigen of interest may also elicit an antibody-mediated immune response. Hence, an immunological response may include one or more of the following effects: the production of antibodies (e.g., IgA or IgG) by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the phrase “immune response to a plurality of strains of influenza virus such as H5 influenza virus, wherein at least one strain of the plurality of strains is not the same strain of said two divergent HAs such as H5 HAs” has its ordinary meaning as known to those skilled in the art and refers to an additional immune response that is induced, not only, for example, two immune responses expected to be induced by two divergent HAs such as H5 HAs. In certain aspects, the phrase refers to a heterologous challenge, meaning the strain of virus used to challenge the protective effect of the vaccine is different from the strain that contains a gene that can encode HAs such as H5 HAs in the DNA construct.

As used herein, the phrase “immune response to a plurality of strains of influenza virus, wherein at least one strain of the plurality of strains does not encode any of the divergent influenza HAs” has its ordinary meaning as known to those skilled in the art and refers to an immune response that includes a response against an influenza strain not represented by the divergent influenza HAs. In certain aspects, the phrase refers to a heterologous challenge, meaning the strain of virus used to challenge the protective effect of the vaccine is different from the strain that contains a gene that can encode HAs in the DNA construct.

A plurality of strains has its ordinary meaning as known to those skilled in the art and includes, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 47, 48, 49, or 50 strains.

As used herein, the phrase “the lifespan of the subject” has its ordinary meaning as known to those skilled in the art and includes at least a substantial portion of the life of a subject. For example, the examples show immunogenicity in immunized mice with respect to challenge by lethal doses of a virus. The mice were immunized at 20 weeks and unexpectedly survived challenge at 68 weeks. A typical lifespan for a mouse is approximately 100 weeks. Thus, the mice in this experiment were immunized until they were 80 weeks old, essentially the approximate remainder of their lifespan after immunization, and survived challenge of lethal H5N1 influenza virus. Thus, in certain aspects, it is expected that the vaccines and compositions described herein will confer immunogenicity for a lifespan of the subject.

In one embodiment, the vaccine and immunogenic compositions described herein are produced using cell-based production technology, and not using egg-based production technology. This method is more efficient and therefore increases the ability to rapidly respond to new emerging virus strains.

Some embodiments described herein concern DNA immunogens and vaccines that contain nucleic acids encoding for influenza hemagglutinin (HA) proteins. The natural viral HA sequence is used in the preparation of some of these compositions. Codon optimization of the HA genes for the particular recipient of the immunogen and/or vaccine is also desired for some embodiments as it may allow for better expression of the at least one construct in the subject and may minimize the chance of homologous recombination with other strains of influenza, which may generate new strains of the virus that could potentially be more pathogenic. Studies have confirmed the minimal chance of host integration and toxicity with codon-optimized constructs. Sheets R L, et al. (2006) Toxicol Sci 91: 610-619; Epstein J E, et al. (2004) Vaccine 22: 1592-1603; Wang Z, et al. (2004) Gene Ther 11: 711-721; Martin J E, et al. (2005) J Allergy Clin Immunol 115: 892.

In other embodiments, DNA immunogens and vaccines that contain nucleic acids encoding for influenza HA proteins are codon-optimized for human expression. These human codon-optimized constructs can be administered to other animals, including horses, pigs, cats, dogs, and farm animals—especially poultry. As human codon-optimized constructs are capable of inducing an immune response in non-human animals, use of the human codon-optimized immunogens and/or vaccines provides the ability to monitor the safety and efficacy of the immunogens and/or vaccines in animals. These data are useful in the face of viral cross-over into the human population where administration of the construct to humans is necessary. Additionally, the human codon-optimized constructs are available for administration to humans without undue modification to allow for more efficient expression.

In some embodiments, an influenza immunogen and/or vaccine that comprises, consists of, or consists essentially of a nucleic acid that encodes at least one influenza HA peptide induces a protective immune response in the host. In other embodiments, an influenza immunogen and/or vaccine that comprises, consists of, or consists essentially of a nucleic acid that encodes at least one influenza HA such as H5 HA peptide induces a protective immune response in the host against a matching live virus challenge (such as would happen in an outbreak) and also elicits a robust protective immune response against a broad range of homologous and heterologous H5 influenza strains. In certain embodiments, an influenza immunogen and/or vaccine that comprises, consists of, or consists essentially of a nucleic acid that encodes at least one influenza HA, such as H1 HA or H3 HA, induces a protective immune response in the host against a matching live virus challenge and also elicits a robust protective immune response against a broad range of homologous and heterologous H1 or H3 influenza strains. Due to the unpredictable antigenic drift of the influenza virus genome, it has been very difficult to predict the next dominant strain of an avian endemic outbreak. A broadly protective vaccine is desirable for this particular reason.

In some embodiments, nucleic acids encoding immunogenic influenza peptides are inserted into DNA constructs capable of expression in the intended host. In some embodiments, each nucleic acid encoding a specific influenza peptide is inserted into a separate DNA construct. In other embodiments, nucleic acids encoding multiple influenza peptides are inserted into the same expression vector, an example of a DNA construct.

In some embodiments, a DNA construct comprises a nucleic acid encoding at least one influenza HA, such as H1 HA, H3 HA or H5 HA, the expression of which is directed by a CMV or CMV/R vector. Examples of such DNA constructs are provided herein. CMV/R is described in U.S. Pat. No. 7,094,598 B2, issued Aug. 22, 2006.

In other embodiments, nucleic acids encoding immunogenic influenza peptides are inserted into the genome of an adenovirus for efficient delivery to the intended host. In some embodiments, each nucleic acid encoding a specific influenza peptide is inserted into a separate adenoviral vector. In other embodiments, nucleic acids encoding multiple influenza peptides are inserted into the same adenoviral vector.

In a some embodiments, adenovirus-5 is used. In another embodiment, an E-1 deleted version of adenovirus-5 is used. As an example, SEQ ID NO.: 19 depicts an adenovirus-5 containing DNA encoding the HA H5 peptide of A-Indonesia.

A broadly protective murine vaccine can be made by including more H5 HAs from varying strains in a multivalent vaccine (FIG. 1). In some embodiments, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40 or more different HAs are included in a single immunogen and/or vaccine. As it is not always practical to include 10 or even 5 different HAs in a single immunogen and/or vaccine due to the cost and complexity of manufacturing such compositions, some embodiments include an immunogen and/or vaccine that induces broad protection with the inclusion of a minimal number of HAs. In some embodiments, DNA encoding the H5 HA genes presented in Table 1 are used to generate immunogens and/or vaccines that induce an immune response and/or protection in the host against a matching live virus challenge (such as would happen in an outbreak). Some of these embodiments may also elicit a robust protective immune response against a broad range of homologous and heterologous H5 influenza strains:

TABLE 1
H5 HA Sequences

| | | Accession | |
|---|---|---|---|
| Name | Protein Sequence | Number | SEQ ID NO: |
| A/Thailand/ | ICQMEKIVLLFAIVSLVKSDQICIGYHA | AY555150 | SEQ ID NO: 1 |
| 1(KAN-1)/ | NNSTEQVDTIMEKNVTVTHAQDILEKTH | | |
| 2004 | NGKLCDLDGVKPLILRDCSVAGWLLGNP | | |
| | MCDEFINVPEWSYIVEKANPVNDLCYPG | | |
| | DFNDYEELKHLLSRINHFEKIQIIPKSS | | |
| | WSSHEASLGVSSACPYQRKSSFFRNVVW | | |
| | LIKKNSTYPTIKRSYNNTNQEDLLVLWG | | |
| | IHHPNDAAEQTKLYQNPTTYISVGTSTL | | |
| | NQRLVPRIATRSKVNGQSGRMEFFWTIL | | |
| | KPNDAINFESNGNFIAPEYAYKIVKKGD | | |
| | STIMKSELEYGNCNTKCQTPMGAINSSM | | |
| | PFHNIHPLTIGECPKYVKSNRLVLATGL | | |
| | RNSPQRERRRKKRGLFGAIAGFIEGGWQ | | |
| | GMVDGWYGYHHSNEQGSGYAADKESTQK | | |
| | AIDGVTNKVNSIIDKMNTQFEAVGREFN | | |
| | NLERRIENLNKKMEDGFLDVWTYNAELL | | |
| | VLMENERTLDFHDSNVKNLYDKVRLQLR | | |
| | DNAKELGNGCFEFYHKCDNECMESVRNG | | |
| | STYDYPQYEEARLKREEISGVKLESIGI | | |
| | SYQILSIYTVASSLALAIMVAGLSLWMC | | |
| | SNGSLQCRICI | | |
| A/Vietnam/ | MEKIVLLFAIVSLVKSDQICIGYHANNS | AY651334 | SEQ ID NO: 2 |
| 1203/2004 | TEQVDTIMEKNVTVTHAQDILEKKHNGK | | |
| | LCDLDGVKPLILRDCSVAGWLLGNPMCD | | |
| | EFINVPEWSYIVEKANPVNDLCYPGDFN | | |
| | DYEELKHLLSRINHFEKIQIIPKSSWSS | | |
| | HEASLGVSSACPYQGKSSFFRNVVWLIK | | |
| | KNSTYPTIKRSYNNTNQEDLLVLWGIHH | | |
| | PNDAAEQTKLYQNPTTYISVGTSTLNQR | | |
| | LVPRIATRSKVNGQSGRMEFFWTILKPN | | |
| | DAINFESNGNFIAPEYAYKIVKKGDSTI | | |
| | MKSELEYGNCNTKCQTPMGAINSSMPFH | | |
| | NIHPLTIGECPKYVKSNRLVLATGLRNS | | |
| | PQRERRRKKRGLFGAIAGFIEGGWQGMV | | |
| | DGWYGYHHSNEQGSGYAADKESTQKAID | | |
| | GVTNKVNSIIDKMNTQFEAVGREFNNLE | | |
| | RRIENLNKKMEDGFLDVWTYNAELLVLM | | |
| | ENERTLDFHDSNVKNLYDKVRLQLRDNA | | |
| | KELGNGCFEFYHKCDNECMESVRNGTYD | | |
| | YPQYSEEARLKREEISGVKLESIGIYQI | | |
| | LSIYSTVASSLALAIMVAGLSLWMCSNG | | |
| | SLQCR | | |
| A/Hong | MEKTVLLLATVSLVKSDQICIGYHANNS | AAC32088 | SEQ ID NO: 3 |
| Kong/ | TEQVDTIMEKNVTVTHAQDILERTHNGK | | |
| 156/1997 | LCDLNGVKPLILRDCSVAGWLLGNPMCD | | |
| | EFINVPEWSYIVEKASPANDLCYPGNFN | | |
| | DYEELKHLLSRINHFEKIQIIPKSSWSN | | |
| | HDASSGVSSACPYLGRSSFFRNVVWLIK | | |
| | KNSAYPTIKRSYNNTNQEDLLVLWGIHH | | |
| | PNDAAEQTKLYQNPTTYISVGTSTLNQR | | |
| | LVPEIATRPKVNGQSGRMEFFWTILKPN | | |
| | DAINFESNGNFIAPEYAYKIVKKGDSTI | | |
| | MKSELEYGNCNTKCQTPMGAINSSMPFH | | |
| | NIHPLTIGECPKYVKSNRLVLATGLRNT | | |
| | PQRERRRKKRGLFGAIAGFIEGGWQGMV | | |
| | DGWYGYHHSNEQGSGYAADKESTQKAID | | |
| | GVTNKVNSIINKMNTQFEAVGREFNNLE | | |
| | RRIENLNKKMEDGFLDVWTYNAELLVLM | | |
| | ENERTLDFHDSNVKNLYDKVRLQLRDNA | | |
| | KELGNGCFEFYHKCDNECMESVKNGTYD | | |
| | YPQYSEEARLNREEISGVKLESMGTYQI | | |
| | LSIYSTVASSLALAIMVAGLSLWMCSNG | | |
| | SLQCRICI | | |
| A/Hong | MEKIVLLLATVSLVKSDQICIGYHANNS | AAC32099.1 | SEQ ID NO: 4 |
| Kong/ | TEQVDTIMEKNVTVTHAQDILERTHNGK | | |
| 483/1997 | LCDLNGVKPLILRDCSVAGWLLGNPMCD | | |
| | EFINVPEWSYIVEKASPANDLCYPGNFN | | |
| | DYEELKHLLSRISHFEKIQIIPKSSWSN | | |
| | HDASSGVSSACPYLGKSSFFRNVVWLIK | | |
| | KNSTYPTIKRSYNNTNQEDLLVLWGIHH | | |
| | PNDAAEQTKLYQNPTTYISVGTSTLNQR | | |
| | LVPEIATRPKVNGQSGRIEFFWTILKPN | | |
| | DAINFESNGNFIAPEYAYKIVKKGDSTI | | |
| | MKSELEYGNCNTKCQTPMGAINSSMPFH | | |
| | NIHPLTIGECPKYVKSNRLVLATGLRNA | | |
| | PQRERRRKKRGLFGAIAGFIEGGWQGMV | | |
| | DGWYGYHHSNEQGSGYAADQESTQKAID | | |
| | GVTNKVNSIINKMNTQFEAVGREFNNLE | | |
| | RRIENLNKKMEDGFLDVWTYNAELLVLM | | |
| | ENERTLDFHDSNVKNLYDKVRLQLRDNA | | |
| | KELGNGCFEFYHKCDNECMESVKNGTYD | | |
| | YPQYSEEARLNREEISGVKLESMGTYQI | | |
| | LSLYSTVASSLALAIMVAGLSLWMCSNG | | |
| | SLQCRICI | | |
| A/chicken/ | MEKIVLLLAIVSLVKSDQICIGYHANNS | AAV97603.1 | SEQ ID NO: 5 |
| Korea/ | TEQVDTIMEKNVTVTHAQDILEKTHNGK | | |
| ES/2003 | LCDLDGVKPLILRDCSVAGWLLGNPMCD | | |
| | EFINVPEWSYIVEKANPPNDLCYPGNFN | | |
| | DYEELKHLLSRINHFEKIQIIPKSSWSD | | |
| | HEASSGVSSACPYQGRSSFFRNVVWLIK | | |
| | KNSAYPTIKRSYNNTNQEDLLVLWGIHH | | |
| | PNDAAEQTRLYQNPTTYISVGTSTLNQR | | |
| | LVPKIATRSKVNGQSGRMEFFWTILKPN | | |
| | DAISFESNGNFIAPEYAYKIVKKGDSAI | | |
| | MKSELEYGNCNTKCQTPMGAINSSMPFH | | |
| | NIHPLTIGECPKYVKSSRLVLATGLRNS | | |
| | PQREKRKKRGLFGAIAGFIEGGWQGMVD | | |
| | GWYGYHHSNEQGSGYAADKESTQKAIDG | | |
| | VTNKVNSIIDKMNTQFEAVGREFNNLER | | |
| | RIENLNKKMEDGFLDVWTYNAELLVLME | | |
| | NERTLDFHDSNVKNLYDKVRLQLRDNAK | | |
| | ELGNGCFEFYHRCDNECIESVRNGTYGY | | |
| | PQYSEEARLKREEISGVKLESIGTYQIL | | |
| | SIYSTVASSLALAIMVAGLSLWMCSNGS | | |
| | LQCRICI | | |
| A/ | MEKIVLLLAIVSLVKSDQICIGYHANNS | ISDN125873 | SEQ ID NO: 6 |
| Indonesia/ | TEQVDTIMEKNVTVTHAQDILEKTHNGK | | |
| 05/2005 | LCDLDGVKPLILRDCSVAGWLLGNPMCD | | |
| | EFINVPEWSYIVEKANPTNDLCYPGSFN | | |
| | DYEELKHLLSRINHFEKIQIIPKSSWSD | | |
| | HEASSGVSSACPYLGSPSFFRNVVWLIK | | |
| | KNSTYPTIKKSYNNTNQEDLLVLWGIHH | | |
| | PNDAAEQTRLYQNPTTYISIGTSTLNQR | | |
| | LVPKIATRSKVNGQSGRMEFFWTILKPN | | |
| | DAINFESNGNFIAPEYAYKIVKKGDSAI | | |
| | MKSELEYGNCNTKCQTPMGAINSSMPFH | | |
| | NIHPLTIGECPKYVKSNRLVLATGLRNS | | |
| | PQRESRRKKRGLFGAIAGFIEGGWQGMV | | |
| | DGWYGYHHSNEQGSGYAADKESTQKAID | | |
| | GVTNKVNSIIDKMNTQFEAVGREFNNLE | | |
| | RRIENLNKKMEDGFLDVWTYNAELLVLM | | |
| | ENERTLDFHDSNVKNLYDKVRLQLRDNA | | |
| | KELGNGCFEFYHKCDNECMESIRNGTYN | | |
| | YPQYSEEARLKREEISGVKLESIGTYQI | | |
| | LSIYSTVASSLALAIMMAGLSLWMCSNG | | |
| | SLQCRICI | | |
| A/Turkey/ | MEKIVLLLAIVSLVKSDQICIGYHANNS | DQ407519 | SEQ ID NO: 7 |
| Turkey/ | TEQVDTIMEKNVTVTHAQDILEKTHNGK | | |
| 1/2005 | LCDLDGVKPLILRDCSVAGWLLGNPMCD | | |
| | EFLNVPEWSYIVEKINPANDLCYPGNFN | | |
| | DYEELKHLLSRINHFEKIQIIPKSSWSD | | |
| | HEASAGVSSACPYQGRSSFFRNVVWLIK | | |
| | KDNAYPTIKRSYNNTNQEDLLVLWGIHH | | |
| | PNDAAEQTRLYQNPTTYISVGTSTLNQR | | |
| | LVPKIATRSKVNGQSGRMEFFWTILKPN | | |
| | DAINFESNGNFIAPENAYKIVKKGDSTI | | |
| | MKSELEYGNCNTKCQTPIGAINSSMPFH | | |
| | NIHPLTIGECPKYVKSSRLVLATGLRNS | | |
| | PQGERRRKKRGLFGAIAGFIEGGWQGMV | | |
| | DGWYGYHHSNEQGSGYAADKESTQKAID | | |
| | GVTNKVNSIIDKMNTQFEAVGREFNNLE | | |
| | RRIENLNKKMEDGFLDVWTYNAELLVLM | | |
| | ENERTLDFHDSNVKNLYDKVRLQLRDNA | | |
| | KELGNGCFEFYHRCDNECMESVRNGTYD | | |
| | YPQYSEEARLKREEISGVKLESIGTYQI | | |
| | LSIYSTVASSLALAIMVAGLSLWMCSNG | | |
| | SLQCRICI | | |
| A/Egypt/ | ICIGYHANNSTEQVDTIMEKNVTVTHAQ | ABE01046 | SEQ ID NO: 8 |
| 2782- | DILEKTHNGKLCDLDGVKPLILRDCSVA | | |
| NAMRU3/ | GWLLGNPMCDEFLNVPEWSYIVEKINPA | | |
| 2006 | NDLCYPGNFNDYEELKHLLSRINHFEKI | | |
| | QIIPKSSWSDHEASSGVSSACPYQGRSS | | |
| | FFRNVVWLIKKDNAYPTIKRSYNNTNQE | | |
| | DLLVLWGIHHPNDAAEQTRLYQNPTTYI | | |
| | SVGTSTLNQRLVPKIATRSKVNGQSGRM | | |
| | EFFWTILKSNDAINFESNGNFIAPENAY | | |
| | KIVKKGDSTIMKSELEYGNCNTKCQTPI | | |
| | GAINSSMPFHNIHPLTIGECPKYVKSNR | | |
| | LILATGLRNSPQGERRRKKRGLFGAIAG | | |
| | FIEGGWQGMVDGWYGYHHSNEQGSGYAA | | |
| | DKESTQKAIDGVTNKVNSIIDKMNTQFE | | |
| | AVGREFNNLERRIENLNKKMEDGFLDVW | | |
| | TYNAELLVLMENERTLDFHDSNVKNLYD | | |
| | KVRLQLRDNAKELGNGCFEFYHRCDNEC | | |
| | MESVRNGTYDYPQYSEEARLKREEISGV | | |
| | KLESIGTYQILSIYSTVASSLALAIMVA | | |
| | GLFLWMCSNGSLQC | | |
| A/chicken/ | VLLLAIVSLVKSDQICIGYHANNSTEQV | DQ406728 | SEQ ID NO: 9 |
| Nigeria/ | DTIMEKNVTVTHAQDILEKTHNGKLCDL | | |
| 641/2006 | DGVKPLILRDCSVAGWLLGNPMCDEFLN | | |
| | VPEWSYIVEKINPANDLCYPGNFNDYEE | | |
| | LKHLLSRINHFEKIQIIPKSSWSDHEAS | | |
| | SGVSSACPYQGRSSFFRNVVWLIKKDNA | | |
| | YPTIKRSYNNTNQEDLLVLWGIHHPNDA | | |
| | AEQTRLYQNPTTYISVGTSTLNQRLVPK | | |
| | IATRSKVNGQSGRMEFFWTILKPNDAIN | | |
| | FESNGNFIAPENAYKIVKKGDSTIMKSE | | |
| | LEYGNCNTKCQTPIGAINSSMPFHNIHP | | |
| | LTIGECPKYVKSNRLVLATGLRNSPQGE | | |
| | RRRKKRGLFGAIAGFIEGGWQGMVDGWY | | |
| | GYHHSNEQGSGYAADKESTQKAIDGVTN | | |
| | KVNSIIDKMNTQFEAVGREFNNLERRIE | | |
| | NLNKKMEDGFLDVWTYNAELLVLMENER | | |
| | TLDFHDSNVKNLYDKVRLQLRDNAKELG | | |
| | NGCFEFYHRCDNECMESVRNGTYDYPQY | | |
| | SEEARLKREEISGVKLESIGTYQILSIY | | |
| | STVASSLALAIMVAGLSLWMCSNG | | |
| A/Iraq/ | DQICIGYHANNSTEQVDTIMEKNVTVTH | DQ435202 | SEQ ID NO: 10 |
| 207- | AQDILEKTHNGKLCDLDGVKPLILRDCS | | |
| NAMRU3/ | VAGWLLGNPMCDEFLNVPEWSYIVEKIN | | |
| 2006 | PANDLCYPGNFNDYEELKHLLSRINHFE | | |
| | KIQIIPKSSWSDHEASSGVSSACPYQGR | | |
| | SSFFRNVVWLIKKDNAYPTIKRSYNNTN | | |
| | QEDLLVLWGIHHPSDAAEQTRLYQNPTT | | |
| | YISVGTSTLNQRLVPKIATRSKVNGQSG | | |
| | RMEFFWTILKPNDAINFESNGNFIAPEN | | |
| | AYKIVKKGDSTIMKSELEYGNCNTKCQT | | |
| | PIGAINSSMPFHNIHPLTIGECPKYVKS | | |
| | NRLVLATGLRNSPQGERRRKKRGLFGAI | | |
| | AGFIEGGWQGMVDGWYGYHHSNEQGSGY | | |
| | AADKESTQKAIDGVTNKVNSIIDKMNTQ | | |
| | FEAVGREFNNLERRIENLNKKMEDGFLD | | |
| | VWTYNAELLVLMENERTLDFHDSNVKNL | | |
| | YDKVRLQLRDNAKELGNGCFEFYHRCDN | | |
| | ECMESVRNGTYDYPQYSEEARLKREEIS | | |
| | GVKLESIGTYQILSIYSTVASSLALAIM | | |
| | VAGLSLWMCSNGSLQCK | | |
| A/Anhui/ | MEKIVLLLAIVSLVKSDQICIGYHANNS | ABD28180 | SEQ ID NO: 11 |
| 1/2005 | TEQVDTIMEKNVTVTHAQDILEKTHNGK | | |
| | LCDLDGVKPLILRDCSVAGWLLGNPMCD | | |
| | EFINVPEWSYIVEKANPANDLCYPGNFN | | |
| | DYEELKHLLSRINHFEKIQIIPKSSWSD | | |
| | HEASSGVSSACPYQGTPSFFRNVVWLIK | | |
| | KNNTYPTIKRSYNNTNQEDLLILWGIHH | | |
| | SNDAAEQTKLYQNPTTYISVGTSTLNQR | | |
| | LVPKIATRSKVNGQSGRMDFFWTILKPN | | |
| | DAINFESNGNFIAPEYAYKIVKKGDSAI | | |
| | VKSEVEYGNCNTKCQTPIGAINSSMPFH | | |
| | NIHPLTIGECPKYVKSNKLVLATGLRNS | | |
| | PLRERRRKRGLFGAIAGFIEGGWQGMVD | | |
| | GWYGYHHSNEQGSGYAADKESTQKAIDG | | |
| | VTNKVNSIIDKMNTQFEAVGREFNNLER | | |
| | RIENLNKKMEDGFLDVWTYNAELLVLME | | |
| | NERTLDFHDSNVKNLYDKVRLQLRDNAK | | |
| | ELGNGCFEFYHKCDNECMESVRNGTYDY | | |
| | PQYSEEARLKREEISGVKLESIGTYQIL | | |
| | SIYSTVASSLALAIMVAGLSLWMCSNGS | | |
| | LQCRICI | | |

A broadly protective immunogen or vaccine can be made by including at least one H1 HA or H3 HA from varying strains in a multivalent vaccine. In some embodiments, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40 or more different H1 HAs and/or H3 HAs are included in a single immunogen and/or vaccine. As it is not always practical to include 10 or even 5 different HAs in a single immunogen and/or vaccine due to the cost and complexity of manufacturing such compositions, some embodiments include an immunogen and/or vaccine that induces broad protection with the inclusion of a minimal number of H1 HAs and/or H3 HAs. In some embodiments, one or more DNA constructs encoding at least two divergent H1 HAs and/or H3 HAs of the HAs presented in Table 2 are used to generate immunogens and/or vaccines that induce an immune response and/or protection in the host against a matching live virus challenge. Some of these embodiments may also elicit a robust protective immune response against a broad range of homologous and heterologous H5 influenza strains.

TABLE 2
H1 HA and H3 HA Sequences

| Name | Protein Sequence | SEQ ID NO |
|---|---|---|
| A/swine/Ohio/ | MKAILVVLLYTFTTANADTLCIGYH | 30 |
| 51145/2007 | ANNSTDTVDTVLEKNVTVTHSVNLL | |
| (H1N1) | ENRHNGKLCKLRGVAPLHLGKCNIA | |
| | GWLLGNPECESLSTASSWSYIVETS | |
| | NSDNGTCYPGDFINYEELREQLSSV | |
| | SSFERFEIFPKTSSWPNHDTNRGVT | |
| | AACPHAGTNSFYRNLIWLVKKGNSY | |
| | PKINKSYINNKEKEVLVLWAIHHPS | |
| | TSADQQSLYQNADAYVFVGSSRYSR | |
| | KFEPEIATRPKVRDQAGRMNYYNTL | |
| | VEPGDKITFEATGNLVVPRYAFALK | |
| | RNSGSGTITSDTSVHDCDTTCQTPN | |
| | GAINTSLPFQNIHPVTIGECPKYVK | |
| | STKLRMATGLRNIPSIQSRGLFGAI | |
| | AGFIEGGWTGMIDGWYGYHHQNEQG | |
| | SGYAADLKSTQNAIDGITNKVNSVI | |
| | EKMNTQFTAVGKEFSHLERRIENLN | |
| | KKVDDGFLDIWTYNAELLVLLENER | |
| | TLDYHDSNVKNLYEKVRSQLKNNAK | |
| | EIGNGCFEFYHKCDDTCMESVKNGT | |
| | YDYPKYSEEAKLNREEIDGVKLEST | |
| | RIYQILAIYSTVASSLVLVVSLGAI | |
| | SFWMCSNGSLQCRICI | |
| A/swine/North | MKTIIAFSYILCLIFAQKLPGSDNS | 32 |
| Carolina/R08- | MATLCLGHHAVPNGTLVKTITDDQI | |
| 001877/2008 | EVTNATELVQSSSTGRICNSPHQIL | |
| (H3N2) | DGKNCTLIDALLGDPHCDDFQNKEW | |
| | DLFVERSTAYSNCYPYYVPDYASLR | |
| | SLVASSGTLEFTQESFNWTGVAQDG | |
| | SSYACRRKSVNSFFSRLNWLHNLNY | |
| | KYPALNVTMPNNDKFDKLYIWGVHH | |
| | PGTDRDQTNLYVQASGRVTVSTKRS | |
| | QQTVIPNIGSRPWVRGVSSIISIYW | |
| | TIVKPGDILLINSTGNLIAPRGYFK | |
| | IQSGKSSIMRSDAPIGNCNSECITP | |
| | NGSIPNDKPFQNVNRITYGACPRYV | |
| | KQNTLKLATGMRNVPEKQTRGIFGA | |
| | IAGFIENGWEGMVDGWYGFRHQNSE | |
| | GTGQAADLKSTQAAVNQITGKLNRV | |
| | IKKTNEKFHQIEKEFSEVEGRIQDL | |
| | EKYVEDTKIDLWSYNAELLVALENQ | |
| | HTIDLTDSEMNKLFERTRKQLRENA | |
| | EDMGNGCFKIYHKCDNACIGSIRNG | |
| | TYDHDVYRDEALNNRFQIKGVQLKS | |
| | GYKDWILWISFAISCFLLCVVLLGF | |
| | IMWACQKGNIRCNICI | |
| A/swine/ | MKAILVVLLYTFATANADTLCIGYH | 34 |
| California/ | ANNSTDTVDTVLEKNVTVTHSVNLL | |
| 04/09(H1N1) | EDKHNGKLCKLRGVAPLHLGKCNIA | |
| | GWILGNPECESLSTASSWSYIVETP | |
| | SSDNGTCYPGDFIDYEELREQLSSV | |
| | SSFERFEIFPKTSSWPNHDSNKGVT | |
| | AACPHAGAKSFYKNLIWLVKKGNSY | |
| | PKLSKSYINDKGKEVLVLWGIHHPS | |
| | TSADQQSLYQNADTYVFVGSSRYSK | |
| | KFKPEIAIRPKVRDQEGRMNYYWTL | |
| | VEPGDKITFEATGNLVVPRYAFAME | |
| | RNAGSGIIISDTPVHDCNTTCQTPK | |
| | GAINTSLPFQNIHPITIGKCPKYVK | |
| | STKLRLATGLRNIPSIQSRGLFGAI | |
| | AGFIEGGWTGMVDGWYGYHHQNEQG | |
| | SGYAADLKSTQNAIDEITNKVNSVI | |
| | EKMNTQFTAVGKEFNHLEKRIENLN | |
| | KKVDDGFLDIWTYNAELLVLLENER | |
| | TLDYHDSNVKNLYEKVRSQLKNNAK | |
| | EIGNGCFEFYHKCDNTCMESVKNGT | |
| | YDYPKYSEEAKLNREEIDGVKLEST | |
| | RIYQILAIYSTVASSLVLVVSLGAI | |
| | SFWMCSNGSLQCRICI | |
| A/equine/Ohio/ | MKTTIILILLTHWAYSQNPISGNNT | 36 |
| 1/2003(H3N8) | ATLCLGHHAVANGTLVKTISDDQIE | |
| | VTNATELVQSISMGKICNNSYRILD | |
| | GRNCTLIDAMLGDPHCDAFQYENWD | |
| | LFIERSSAFSNCYPYDIPDYASLRS | |
| | IVASSGTLEFTAEGFTWTGVTQNGR | |
| | SGACKRGSADSFFSRLNWLTKSGSS | |
| | YPTLNVTMPNNKNFDKLYIWGIHHP | |
| | SSNQEQTKLYIQESGRVTVSTKRSQ | |
| | QTIIPNIGSRPWVRGQSGRISIYWT | |
| | IVKPGDILMINSNGNLVAPRGYFKL | |
| | KTGKSSVMRSDVPIDICVSECITPN | |
| | GSISNDKPFQNVNKVTYGKCPKYIR | |
| | QNTLKLATGMRNVPEKQIRGIFGAI | |
| | AGFIENGWEGMVDGWYGFRYQNSEG | |
| | TGQAADLKSTQAAIDQINGKLNRVI | |
| | ERTNEKFHQIEKEFSEVEGRIQDLE | |
| | KYVEDTKIDLWSYNAELLVALENQH | |
| | TIDLTDAEMNKLFEKTRRQLRENAE | |
| | DMGGGCFKIYHKCDNACIGSIRNGT | |
| | YDHYIYRDEALNNRFQIKGVELKSG | |
| | YKDWILWISFAISCFLICVVLLGFI | |
| | MWACQKGNIRCNICI | |
| A/equine/Bari/ | MKTTIIFIFILLTHWAYSQNPISDN | 38 |
| 2005 H3N8 | NTATLCLGHHAVANGTLVKTISDDQ | |
| | IEVTNATELVQSISMGKICNNSYRI | |
| | LDGRNCTLIDAMLGDPHCDVFQYEN | |
| | WDLFIERSSAFSNCYPYDIPDYASL | |
| | RSIVASSGTLEFTAEGFTWTGVTQN | |
| | GRSGACKRGSADSFFSRLNWLTKSG | |
| | NSYPTLNVTMPNNKNFDKLYIWGIH | |
| | HPSSNQEQTKLYIQESGRVTVSTKR | |
| | SQQTMIPNIGSRPWVRGQSGRISIY | |
| | WTIVKPGDILMINSNGNLVAPRGYF | |
| | KLKTGKSSVMRSDVPIDICVSECIT | |
| | PNGSISNDKPFQNVNKVTYGKCPKY | |
| | IRQNTLKLATGMRNVPEKQIRGIFG | |
| | AIAGFIENGWEGMVDGWYGFRYQNS | |
| | EGTGQAADLKSTQAAIDQINGKLNR | |
| | VIERTNEKFHQIEKEFSEVEGRIQD | |
| | LEKYVEDTKIDLWSYNAELLVALEN | |
| | QHTIDLTDAEMNKLFEKTRRQLREN | |
| | AEDMGGGCFKIYHKCDNACIGSIRN | |
| | GTYDHYIYRDEALNNRFQIKGVELK | |
| | SGYKDWILWISFAISCFLICVVLLG | |
| | FIMWACQKGNIRCNICI | |
| A/equine/ | METTIILILLTHWVYSQNPISGNNT | 40 |
| Aboyne/1/05 | ATLCLGHHAVANGTLVKTITDDQIE | |
| (H3N8) | VTNATELVESISMGKICNNSYRVLD | |
| | GRNCTLIDAMLGDPHCDDFQYESWD | |
| | LFIERSSASSNCYPYDIPDYASLRS | |
| | IVASSGTLEFTAEGFTWTGVTQNGR | |
| | SGACKRGSADSFFSRLNWLTKSGNS | |
| | YPTLNVTMPNNKNFDKLYIWGIHHP | |
| | SSNKEQTKLYIQESGRVTVSTERSQ | |
| | QTVIPNIGSRPWVRGQSGRISIYWT | |
| | IVKPGDVLMINSNGNLVAPRGYFKL | |
| | RTGKSSVMRSDALIDTCVSECITPN | |
| | GSIPNDKPFQNVNKITYGRCPKYIR | |
| | QNTLKLATGMRNVPEKQIRGIFGAI | |
| | AGFIENGWEGMVDGWYGFRYQNSEG | |
| | TGQAADLKSTQAAIDQINGKLNRVI | |
| | ERTNEKFHQIEKEFSEVEGRIQDLE | |
| | KYVEDTKIDLWSYNAELLVALENQH | |
| | TIDLTDAEMNKLFERTRRQLRENAE | |
| | DMGGGCFKIYHKCDNACIGSIRNGT | |
| | YDHYIYRDEALNNRFQIKGVELKSG | |
| | YKDWILWISFAISCFLICVVLLGFI | |
| | MWACQKGNIRCNICI | |

In certain embodiments, an immunogen or vaccine encodes one or more of the following influenza HA amino acid sequences: SEQ ID NO:30, SEQ ID NO:32, SEQ ID NO:34, SEQ ID NO:36, SEQ ID NO:38, or SEQ ID NO:40. In certain embodiments, an immunogen or vaccine comprises one or more of the following nucleic acid sequences: SEQ ID NO:29, SEQ ID NO:31, SEQ ID NO:33, SEQ ID NO:35, SEQ ID NO:37, or SEQ ID NO:39. In certain embodiments, an immunogen or vaccine comprises one or more of the following nucleic acid sequences: SEQ ID NO:41, SEQ ID NO:42, SEQ ID NO:43, SEQ ID NO:44, SEQ ID NO:45, SEQ ID NO:46.

Immunogens and/or vaccines that comprise, consist of, or consist essentially of a nucleic acid that encodes a desired HA peptide can be prepared in accordance with conventional methods in molecular biology. These compositions can be formulated for administration to animals, including horses, cats, dogs, pigs and other farm animals—especially poultry, as well as humans that have been identified as a subject in need of an immune response to avian, equine, or swine influenza. In one embodiment, such compositions can be formulated such that the same formulation can be administered to multiple susceptible species. Such subjects can be identified as ones in need of an immune response to avian, equine or swine influenza by clinical or farm practices as known in the art, including, but not limited to diagnostic procedures and observation or evaluation by a health care or veterinary practitioner. In preferred embodiments, immunogens and/or vaccines comprising, consisting of, or consisting essentially of a nucleic acid encoding the desired HA peptide can be prepared as described above for administration to animals susceptible to highly pathogenic avian influenza virus of type A of subtype H5N1 (HPAI A(H5N1)), including, but not limited to: chickens (Gallus domesticus); galliformes including turkeys, grouse, chickens, quails, and pheasants; wood ducks (Aix sponsa); falcons; laughing gulls; and geese. In another embodiment vaccines containing nucleic acid encoding the desired HA peptide can be prepared in accordance with conventional methods for administration to animals known to have been infected with highly pathogenic avian influenza H5N1, including, but not limited to, greylag goose (A. anser) (domestic), whooper swan (Cygnus cygnus); eurasian wigeon (A. penelope), mallard (A. platyrhynchos) (domestic and wild), common pochard (Aythya ferina), tufted duck (Aythya fuligula), smew (Mergellus albellus), jungle fowl (Gallus gallus) (domestic), pheasants (Phasianidae), quail (Coturnix coturnix), wild turkey (Meleagris gallopavo) (domestic), gray heron (Ardea cinerea), little egret (Egretta garzetta), black-headed gull (L. ridibundus), parrots (Psittacidae), and rock pigeon (Columba livia) (domestic). In certain embodiments, immunogens and/or vaccines comprising, consisting of, or consisting essentially of a nucleic acid encoding one or more desired HAs can be prepared as described above for administration to subjects susceptible to equine influenza virus. In certain embodiments, immunogens and/or vaccines comprising, consisting of, or consisting essentially of a nucleic acid encoding one or more desired HAs can be prepared as described above for administration to subjects susceptible to swine influenza virus.

An effective amount of the DNA immunogen and/or vaccine can be incorporated into a pharmaceutical composition with or without a carrier. Routes of administration of the vaccine include, but are not limited to, topical, intranasal, intraocular, subcutaneous, intramuscular, transdermal, intradermal, parenteral, gastrointestinal, transbronchial, intra-ovo for poultry (Oshop et al., In Ovo Delivery of DNA to the Avian Embryo. Vaccine, 21: 1275-1281 (2003), expressly incorporated by reference in its entirety) and transalveolar. The embodiments, as described herein, (e.g., compositions that comprise, consist of, consist essentially of nucleic acids that encode SEQ ID NOs: 1, 2, 3, 4, 5, 6, 7, 8, 9, 10, or 11) or an immunogenic fragment thereof (e.g., a fragment that is at least or equal to a nucleic acid encoding 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 250, 300, 350, or 400 consecutive amino acids of SEQ ID NOs: 1, 2, 3, 4, 5, 6, 7, 8, 9, 10, or 11) can be delivered by any modality of DNA vaccination, such as topical, intranasal, intraocular, subcutaneous, transdermal, intradermal, intramuscular, intra-ovo, parenteral, gastrointestinal, transbronchial, transalveolar, in drinking water, eye drop, spray or electroporation (e.g., Medpulsar®). Similarly, compositions that comprise, consist of, or consist essentially of one or more DNA constructs that encode one or more HAs having amino acid sequence SEQ ID NO:30, SEQ ID NO:32, SEQ ID NO:34, SEQ ID NO:36, SEQ ID NO:38, SEQ ID NO:40, or an immunogenic fragment thereof (e.g., a fragment that comprises 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 250, 300, 350, or 400 consecutive amino acids of SEQ ID NO:30, SEQ ID NO:32, SEQ ID NO:34, SEQ ID NO:36, SEQ ID NO:38, or SEQ ID NO:40) can be delivered by any modality of DNA vaccination described herein.

In embodiments, an effective amount of the DNA vaccine can be administered to birds in commercial poultry houses, zoos, or birds in the wilds via drinking water, eye drop or spray.

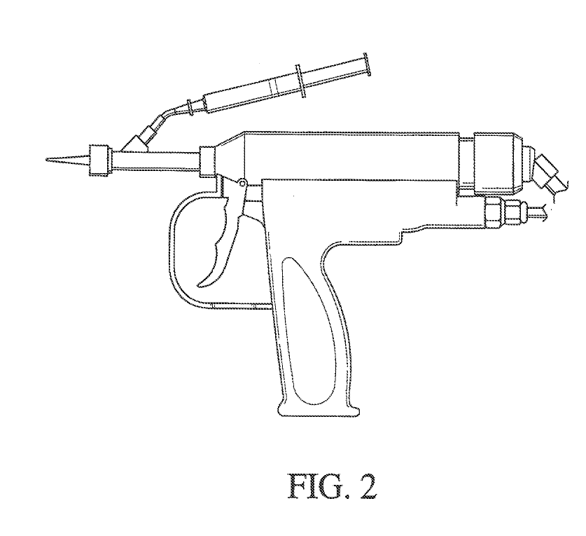

In one embodiment, the immunogens and/or vaccines described herein are provided to the dermis. In some embodiments, the Agro-Jet® needle-free injector is used to deliver an effective amount of the immunogens and/or vaccines described herein to a subject's dermis/subcutaneous tissue so as to provide an intradermal/subcutaneous (ID/SC) inoculation. Agro-Jet® is a needle-free device used for mass delivery of immunogens and/or vaccines and drugs in livestock and poultry (FIG. 2). The device is semi-automatic and uses a small CO₂ tank or compressed air for low-pressure delivery. Upon trigger activation, CO₂ disperses the injectate at a precise dose into the muscle, dermis or subcutaneous tissue depending on the pressure setting of the device. A commercially-available gun is capable of delivering doses ranging from 0.1 ml to 5 ml.

In certain embodiments, other needle-free devices can be used, such as the PHARMAJET® SC/IM Injection System, examples of which are described in the Examples. It is be appreciated that needle-free devices can be used to administer DNA constructs with or without other components, such as adjuvants.

In another preferred embodiment, the immunogens and/or vaccines described herein are sprayed into an area containing the animals in need of an immune response (e.g., a chicken coop, a pen containing farm animals, stable containing horses).

Immunogenic compositions encoding 10 and 5 strain HA proteins in mice were created. In the 5 HA multivalent vaccine testing, protection depended on which 5 HA components were selected. Although both vaccines elicited an immune response that may have been protective heterologously, the animal vaccinated with one of the two 5 multivalent vaccines elicited a better protective immune response against heterologous viral HA. This result is likely attributable to the observation that neutralizing epitopes of different viral strains may vary a great deal. Considering the extensive cost and complications associated with manufacturing a 5 strain version, a trivalent vaccine was developed. Although any HAs disclosed in this application, as well as HAs available in the art, can be used; a trivalent vaccine containing the Vietnam, Indonesia, and Anhui HA was used as the 3-time immunization vaccine candidate in the first chicken homologous virus challenge study. Subsequently, the trivalent DNA HA vaccine including Indonesia, Anhui and Nigeria HAs as a favorable 2-time immunization vaccine was used in a chicken homologous virus challenge study. These three HAs represent a broad range of influenza strains by HA sequence analysis. Bui H H, et al. (2007) Proc Natl Acad Sci USA 104: 246-251.

The disclosure provides immunogens or vaccines that comprise one or more DNA constructs encoding HA from Influenza A/equine/Bari/2005, Influenza A/equine/Ohio/1/2003, Influenza A/equine/Aboyne/1/2005, Influenza A/swine/California/04/2009, Influenza A/swine/Ohio/51145/2007, or Influenza A/swine/North Carolina/R08-001877/2008. Certain embodiments include trivalent equine immunogens or vaccines comprising one or more DNA constructs encoding H3 HA from Influenza A/equine/Bari/2005, Influenza A/equine/Ohio/1/2003, and Influenza A/equine/Aboyne/1/2005. Certain embodiments include trivalent swine immunogens or vaccines comprising one or more DNA constructs encoding H1 HA from Influenza A/swine/California/04/2009, H1 HA from Influenza A/swine/Ohio/51145/2007, and H3 HA from Influenza A/swine/North Carolina/R08-001877/2008.

The disclosure provides immunogens or vaccines that comprise one or more DNA constructs encoding swine H1 HA, H2 HA, H3 HA, H4 HA, H5 HA or H9 HA. Such an HA can be derived from, for example, Influenza A/swine/California/04/2009, Influenza A/swine/Ohio/51145/2007, Influenza A/swine/North Carolina/R08-001877/2008, Influenza A/swine/Illinois/2009, Influenza A/swine/Iowa/03032/2010, Influenza A/swine/Minnesota/03018/2010, Influenza A/swine/England/191973/92, Influenza A/swine/Missouri/4296424/2006, Influenza A/swine/Minnesota/63607-5/2008, Influenza A/swine/Ontario/42729A/01, Influenza A/swine/Anhui/01/2006, Influenza A/swine/Ontario/01911-1/99, Influenza A/swine/Gianyar-Indonesia/07/2006, or Influenza A/swine/Hebei/012/2008.

The disclosure provides immunogens or vaccines that comprise one or more DNA constructs encoding equine H3 HA, H5 HA, or H7 HA. Such an HA can be derived from, for example, Influenza A/equine/Bari/2005, Influenza A/equine/Ohio/1/2003, Influenza A/equine/Aboyne/1/2005, Influenza A/equine/Yokohama/aq19/2009, Influenza A/equine/Egypt/av1/2009, or Influenza A/equine/Newmarket/1/77.

In some embodiments, subjects are provided one or more of the constructs described herein 1, 2, 3, 4, 5, 6, 7, 8, 9, or 10 times so as to elicit an immune response. In a preferred embodiment, the constructs are provided a total of 3 times. In an especially preferred embodiment, the constructs are provided to the subject twice.

In some embodiments, subjects are provided one or more of the constructs provided herein by administration via topical, intranasal, intraocular, subcutaneous, transdermal, intradermal, intramuscular, intra-ovo, parenteral, gastrointestinal, transbronchial, transalveolar, in drinking water, eye drop, spray or electroporation (e.g., Medpulsar) delivery and are subsequently administered with a booster comprising an adenovirus coding for the same immunogens previously administered. In an alternative embodiment, the booster comprises an adenovirus coding for different immunogens than those previously administered.

In certain embodiments, the booster comprises the same immunogen or vaccine as used in the first administration. In certain embodiments, the booster comprises an immunogen or vaccine comprising one or more different DNA constructs than were used in the first administration. Such DNA constructs can be incorporated into a viral vector, such as adenovirus. In certain embodiments, the booster is at least one infectious, inactivated or attenuated influenza virus vaccine.

In other embodiments, subjects are administered with a composition comprising an adenovirus coding for at least one immunogen and are subsequently provided a booster comprising one or more of the constructs provided herein, coding for the same at least one immunogen, by administration via topical, intranasal, intraocular, subcutaneous, transdermal, intradermal, intramuscular, intra-ovo, parenteral, gastrointestinal, transbronchial, transalveolar, in drinking water, eye drop, spray or electroporation (e.g., Medpulsar®) delivery. In an alternative embodiment, the booster comprises one or more of the constructs provided herein, coding for different immunogens than those previously delivered via the adenovirus.

In some embodiments, unhatched eggs are provided one or more of the constructs provided herein by intra-ovo administration and after hatching are subsequently administered with a booster comprising an adenovirus coding for the same immunogens previously administered. In an alternative embodiment, the booster comprises an adenovirus coding for different immunogens than those previously administered.

Two-time DNA inoculation with different doses generated significant immune responses in animals. Unexpectedly, two-time 5 μg trivalent DNA inoculation using the ID/SC route via Agro-Jet® was sufficient to elicit 100% protection against a heterologous viral challenge, whereas the 5 μg trivalent DNA immunization using needle and syringe via IM generated close to 80% protection. Animals vaccinated with the lowest dose, 0.5 μg, showed only 30% protection in IM or ID/SC routes. Thus, lower doses may be used, resulting in a cost savings, for example, for a farmer. Accordingly, Agro-Jet® and traditional needle-syringe routes can both induce similar protective immune responses against a heterologous viral challenge. The use of Agro-Jet® provided better overall protection, however. As such, needle-free immunization is a preferred embodiment for mass immunization programs in humans, horses, cats, dogs, pigs and other farm animals—especially poultry.

The nucleic acids described herein are useful as immunogens and/or vaccines which can be administered alone or in conjunction with an adjuvant. Preferred embodiments include compositions that have as an active ingredient at least one DNA construct that comprise, consist of, or consist essentially of one or more of the nucleic acids described herein with or without an adjuvant. That is, some of the compositions described herein are prepared with or without an adjuvant and comprise, consist, or consist essentially of, as an active ingredient, a nucleic acid encoding an HA protein or a fragment thereof that encodes at least or equal to any number of consecutive amino acids between at least 3-1000 (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 400, 405, 410, 415, 420, 425, 430, 435, 440, 445, 450, 455, 460, 465, 470, 475, 480, 490, 495, 500, 505, 510, 515, 520, 525, 530, 535, 540, 545, 550, 555, 560, 565, 570, 575, 580, 590, 600, 650, 700, 750, 800, 850, 900, 950, or 1000) amino acids in length. Additional compositions are prepared with or without an adjuvant and have an active ingredient that is a DNA that comprises, consists of, or consists essentially of a nucleic acid that encodes one or more HA proteins or a fragment thereof that encodes any number of consecutive amino acids between at least 3-1000 (e.g., 3, 4, 6, 8, 10, 12, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 400, 405, 410, 415, 420, 425, 430, 435, 440, 445, 450, 455, 460, 465, 470, 475, 480, 490, 495, 500, 505, 510, 515, 520, 525, 530, 535, 540, 545, 550, 555, 560, 565, 570, 575, 580, 590, 600, 650, 700, 750, 800, 850, 900, 950, or 1000) amino acids in length. Some of the compositions described herein are prepared with or without an adjuvant and comprise, consist of, or consist essentially of, as an active ingredient, a nucleic acid encoding a mutant HA protein or fragments thereof that encode any number of consecutive amino acids between at least 3-1000 (e.g., 3, 4, 6, 8, 10, 12, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 400, 405, 410, 415, 420, 425, 430, 435, 440, 445, 450, 455, 460, 465, 470, 475, 480, 490, 495, 500, 505, 510, 515, 520, 525, 530, 535, 540, 545, 550, 555, 560, 565, 570, 575, 580, 590, 600, 650, 700, 750, 800, 850, 900, 950, or 1000) amino acids in length. Additional compositions are prepared with or without an adjuvant and comprise, consist of, or consist essentially of, as an active ingredient, a nucleic acid encoding one or more mutant or wild-type HA proteins and fragments thereof that are any number of consecutive amino acids between at least 3-1000 (e.g., at least or equal to 3, 4, 6, 8, 10, 12, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 165, 170, 175, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 400, 405, 410, 415, 420, 425, 430, 435, 440, 445, 450, 455, 460, 465, 470, 475, 480, 490, 495, 500, 505, 510, 515, 520, 525, 530, 535, 540, 545, 550, 555, 560, 565, 570, 575, 580, 590, 600, 650, 700, 750, 800, 850, 900, 950, or 1000) amino acids in length.

In some embodiments, the amino acid and nucleic acid sequences for H5, N1, H1, H3 and other subtypes of influenza, as well as DNA constructs containing such nucleic acid sequences, as disclosed in WO 2007/100584, which was published in English designating the United States, expressly incorporated by reference in its entirety, are used as part of the immunogen and/or vaccine compositions described herein.

The nucleic acid embodiments can also be altered by mutation such as substitutions, additions, or deletions that provide for sequences encoding functionally equivalent molecules. Due to the degeneracy of nucleotide coding sequences, other DNA sequences that encode substantially the same H5 HA amino acid sequence as depicted in SEQ ID NOs: 1-11, or the same H1 HA or H3 HA amino acid sequences as depicted in SEQ ID NO:30, SEQ ID NO:32, SEQ ID NO:34, SEQ ID NO:36, SEQ ID NO:38, or SEQ ID NO:40, or any other HA amino acid sequence can be used in some embodiments. These include, but are not limited to, nucleic acid sequences comprising all or portions of HA peptides or nucleic acids that complement all or part of HA peptides that have been altered by the substitution of different codons that encode a functionally equivalent amino acid residue within the sequence, thus producing a silent change, or a functionally non-equivalent amino acid residue within the sequence, thus producing a detectable change.

The mutant HA nucleic acids used in some of the embodiments described herein also include nucleic acids encoding influenza HA polypeptides or peptides having a non-conservative change that affects the functionality of the molecule. Additional mutants include nucleic acids encoding molecules, wherein the N-terminal region or the C-terminal region is deleted. Further, some mutant nucleic acids encode one or more HA domains combined in a novel fashion so as to create an “HA-like hybrid” molecule, also referred to as a “hybrid”. These hybrids can be used to promote a more robust immune response due to antigenic shuffling for example. Some nucleic acids also encode multimerized HAs or hybrids, which are characterized by a structure having at least two of the same domain (e.g., a hybrid having two epitopes of the regions). Several assays can be employed to evaluate these molecules for their ability to induce an immune response, and many are discussed in detail infra. The HA-like hybrids that are identified for their ability to induce an immune response can be used in biotechnological assays and can be formulated in immunogenic and/or vaccine compositions, as described herein.

The nucleotide sequences encoding the full-length HA proteins, or fragments thereof as described herein, can be modified to generate sequences optimized for expression in human, avian, equine, swine, or other animal cells without altering the encoded polypeptide sequences. Computer algorithms are available for codon optimization. For example, web-based algorithms (e.g., Sharp et al. (1988) Nucleic Acids Res. 16:8207-11, hereby incorporated by reference) can be used to generate a nucleotide sequence with optimized expression in a suitable host (e.g., human, horse, dog, cat, pig, chicken or rodent). As an example, SEQ ID NO.: 17 depicts a human codon-optimized sequence encoding the Influenza H5 HA A-Indonesia peptide and SEQ ID NO.: 18 depicts a chicken codon-optimized sequence encoding the Influenza H5 HA A-Indonesia peptide. Examples of DNA constructs encoding swine H1 HAs that have been codon-optimized include DNA constructs comprising nucleic acid sequences SEQ ID NO:29 and SEQ ID NO:33. Examples of DNA constructs encoding swine or equine H3 HAs that have been codon-optimized include DNA constructs comprising nucleic acid sequences SEQ ID NO:31 (swine), SEQ ID NO:35 (equine), SEQ ID NO:37 (equine) and SEQ ID NO:39 (equine).

Compositions comprising a nucleic acid encoding at least one HA protein or fragment thereof and an adjuvant enhance and/or facilitate an animal's immune response to the antigen. Adjuvant activity is manifested by a significant increase in immune-mediated protection against the antigen, an increase in the titer of antibody raised to the antigen, and an increase in proliferative T cell responses.

Accordingly, compositions (e.g., immunogens, vaccines and other medicaments) that comprise adjuvant and one or more of the nucleic acids described herein are embodiments of the invention. These compositions can vary according to the amount of adjuvant, the form of the adjuvant, as well as the sequence of the nucleic acid. Examples are provided in the Examples section.

Although any adjuvant can be used, preferred embodiments can contain: chemical adjuvants such as aluminum phosphate, benzyalkonium chloride, ubenimex, and QS21; genetic adjuvants such as the IL-2 gene or fragments thereof, the granulocyte macrophage colony-stimulating factor (GM-CSF) gene or fragments thereof, the IL-18 gene or fragments thereof, the chemokine (C-C motif) ligand 21 (CCL21) gene or fragments thereof, the IL-6 gene or fragments thereof, CpG, LPS, TLR agonists, and other immune stimulatory genes; protein adjuvants such IL-2 or fragments thereof, the granulocyte macrophage colony-stimulating factor (GM-CSF) or fragments thereof, IL-18 or fragments thereof, the chemokine (C-C motif) ligand 21 (CCL21) or fragments thereof, IL-6 or fragments thereof, CpG, LPS, TLR agonists and other immune stimulatory cytokines or fragments thereof; lipid adjuvants such as cationic liposomes, N3 (cationic lipid), monophosphoryl lipid A (MPL1); other adjuvants including cholera toxin, enterotoxin, Fms-like tyrosine kinase-3 ligand (Flt-3L), bupivacaine, marcaine, VAXFECTIN (Vical, Inc., San Diego, Calif.), and levamisole. In some embodiments, adenoviruses can be used as adjuvants.

In one preferred embodiment, cationic lipids are used as an adjuvant. In another preferred embodiment, cationic liposomes are used as an adjuvant.

Methods of enhancing or promoting an immune response in an animal including horses, cats, dogs, pigs and other farm animals—especially poultry, as well as humans, to an antigen prepared as described herein are also provided. Such methods can be practiced, for example, by identifying an animal in need of an immune response to influenza and providing said animal a composition comprising one or more of the nucleic acids, as described herein, and, optimally, an amount of adjuvant that is effective to enhance or facilitate an immune response to the antigen/epitope. In some embodiments, the antigen and the adjuvant are administered separately, instead of in a single mixture. Preferably, in this instance, the adjuvant is administered a short time before or a short time after administering the antigen. Preferred methods involve providing the animal in need with a nucleic acid encoding at least one HA, such as H1 HA, H3 HA, or H5 HA peptide, or fragment thereof with or without an adjuvant or a codon-optimized nucleic acid encoding at least one HA, such as H1 HA, H3 HA, or H5 HA peptide, or fragment thereof with or without an adjuvant.

The constructs and methods disclosed herein provide a model for the production of immunogens and/or vaccines against other strains of Influenza, including HA H1, H2, H3, H4, H6, H7, H8, H9, H10, H11, H12, H13, H14, and H15. In some embodiments, nucleic acids encoding at least two distinct peptides from the same strain (e.g., two antigenically distinct HA H1 peptides) are incorporated into DNA constructs as described herein. The immunogen and/or vaccines containing the at least two distinct peptides are administered to an animal and the immune response measured. If the immunogen and/or vaccine coding for at least two distinct peptides elicits an immune response conferring protection against at least three distinct peptides from the same strain (e.g., three antigenically distinct HA H1 peptides), then the immunogen and/or vaccine is considered effective against that particular strain.

Some embodiments include a method of making an immunogenic composition comprising identifying a virus that infects both humans and an animal host, including horses, cats, dogs, pigs, and other farm animals, especially poultry. The animal host is inoculated with an immunogenic composition that comprises a nucleic acid encoding a viral protein or fragment thereof, from the virus. The sera of the animal are analyzed for an immune response against the virus. When an immune response is detected, the nucleic acid encoding the viral protein or fragment thereof, is formulated for introduction into a human.

DNA vaccines offer a generic approach to influenza virus immunization applicable to multiple animal species. In addition, the ability to substitute DNA constructs encoding different strains enables rapid adaptation of the vaccine to newly evolving field isolates. Such DNA constructs can be derived from newly evolving influenza virus or can be the result of reassortment among influenza virus strains. Immunogens and vaccines of the invention have particular utility because they are designed to protect against multiple influenza strains (for example, by including divergent HAs), including strains not represented in the vaccine, thereby allowing for diversified protection, even against new strains that have evolved due to mutation.

The disclosure provides a composition comprising combination immunogens or vaccines. Such a composition includes an immunogen or vaccine comprising one or more DNA constructs that encode at least two divergent influenza HAs as well at least one immunogen or vaccine against another disease. Such an immunogen or vaccine against another disease can comprise at least one nucleic acid construct, can be an active (e.g., attenuated) or inactive (e.g., protein, subunit, inactivated disease-causing agent) immunogen or vaccine, or can be a mixture thereof. Examples of such diseases include other viral diseases, bacterial diseases, parasitic diseases, and the like.

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Standard abbreviations may be used. For example, amino acids can be denoted by either the standard 3-letter or 1-letter code.

### EXAMPLES

DNA vaccines encoding hemagglutinin (HA) proteins from different HPAI H5N1 serotypes were evaluated for their ability to elicit neutralizing antibodies and to protect against homologous and heterologous HPAI H5N1 strain challenge in mice and chickens after DNA immunization by needle and syringe or with a pressure injection device. These vaccines elicited antibodies that neutralized multiple strains of HPAI H5N1 when given in combinations containing up to 10 HAs. The response was dose-dependent, and breadth was determined by the choice of the influenza virus HA in the vaccine. Monovalent and trivalent HA vaccines were tested first in mice and conferred protection against lethal H5N1 A/Vietnam/1203/2004 challenge 68 weeks after vaccination. In chickens, protection was observed against heterologous strains of HPAI H5N1 after vaccination with a trivalent H5 serotype DNA vaccine with doses as low as 5 μg DNA given twice either by intramuscular needle injection or with a needle-free device.

### Example 1

### DNA Constructs

10 different DNA constructs encoding HA from phylogenetically diverse strains of influenza viruses were generated for experiments in mice. DNA constructs encoding different versions of H5 HA protein including SEQ ID NOs: 1-11 were synthesized using human-preferred codons (GeneArt, Regensburg, Germany). Specifically, the H5 HA proteins include (A/Thailand/1(KAN-1)/2004 (clade 1) GenBank AY555150; A/Vietnam/1203/2004 (clade 1) GenBank AY651334; A/Hong Kong/156/1997 (clade 0) GenBank AAC32088; A/Hong Kong/483/1997 GenBank AAC32099.1 (clade 0); A/chicken/Korea/ES/2003 (clade 2.5) GenBank AAV97603.1; A/Indonesia/05/2005 (clade 2.1.3) ISDN125873; A/turkey/Turkey/1/2005 (clade 2.2) GenBank DQ407519; A/Egypt/2782-NAMRU3/2006 (clade 2.2) GenBank ABE01046; A/chicken/Nigeria/641/2006 (clade 2.2) GenBank DQ406728; A/Iraq/207-NAMRU3/2006 (clade 2.2) GenBank DQ435202; A/Anhui/1/2005 (clade 2.3.4) GenBank ABD28180). HA cDNAs from diverse strains of influenza viruses were then inserted into plasmid expression vectors, pCMV/R or pCMV/R 8κB, to form DNA constructs which mediates high level expression and immunogenicity in vivo. For initial trivalent immunizations in chickens, the A/Vietnam/1203/2004, A/Anhui/1/2005 and A/Indonesia/05/2005 strains were used and in the dose response study, the Vietnam strain was replaced with A/chicken/Nigeria/641/2006. The immunogens used in DNA vaccination contained a cleavage site mutation (PQRERRRKKRG (SEQ ID NO.: 12) to PQRETRG (SEQ ID NO.: 13)) as previously described in W. Kong et al., Proc. Natl. Acad. Sci. 103, 15987 (2006) and Yang et al, Science 317, (2007), herein incorporated by reference in their entirety. This mutation was generated by site-directed mutagenesis using a QuickChange kit (Stratagene, La Jolla, Calif.).

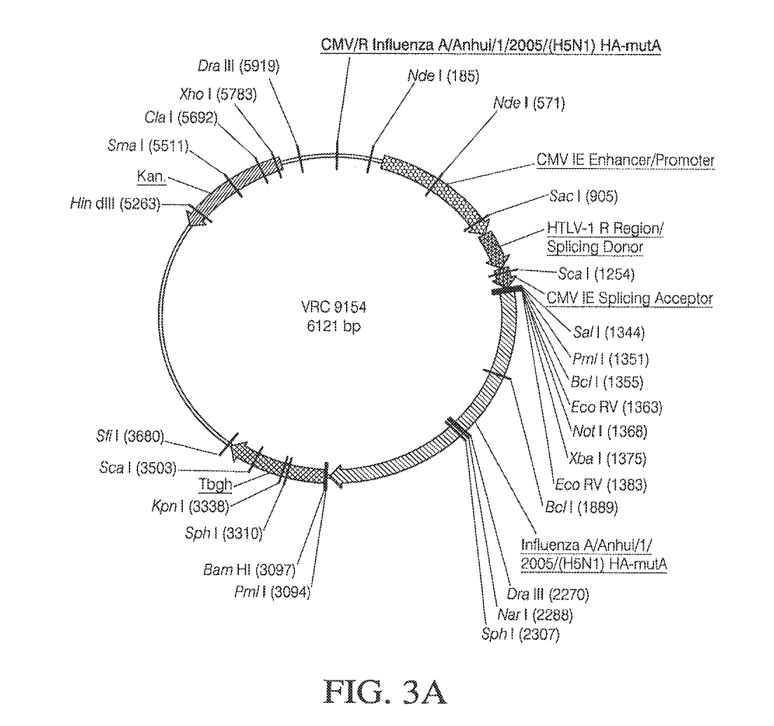

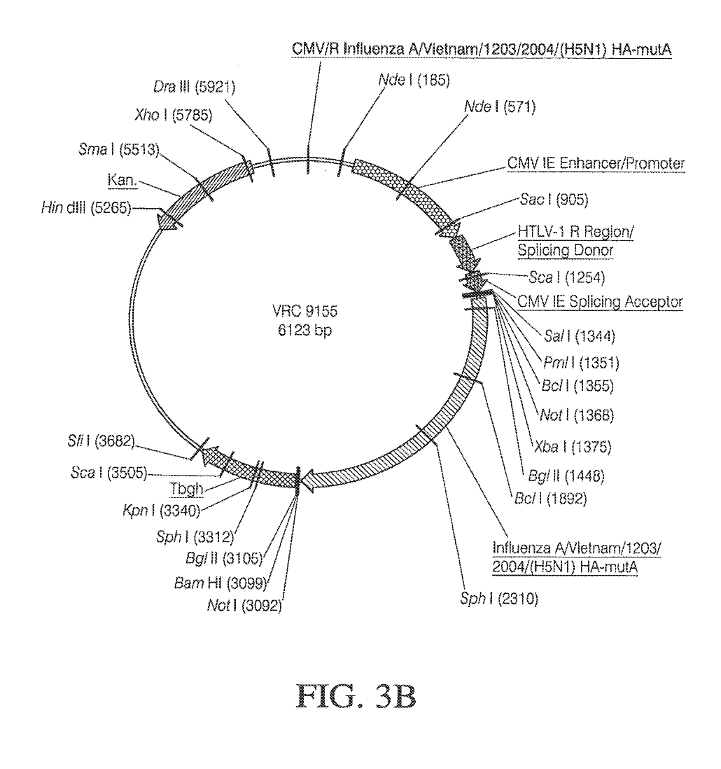

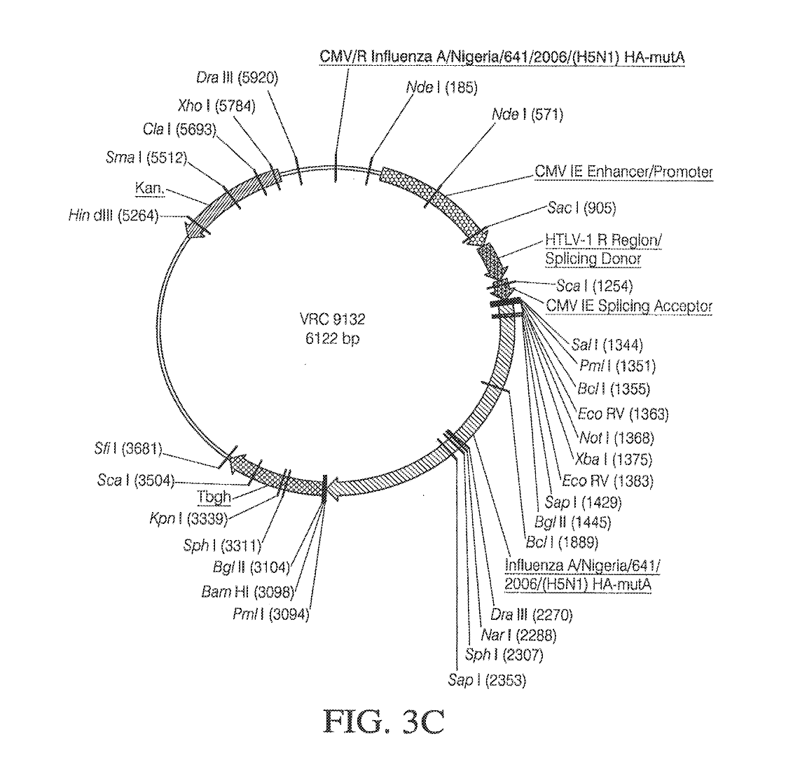

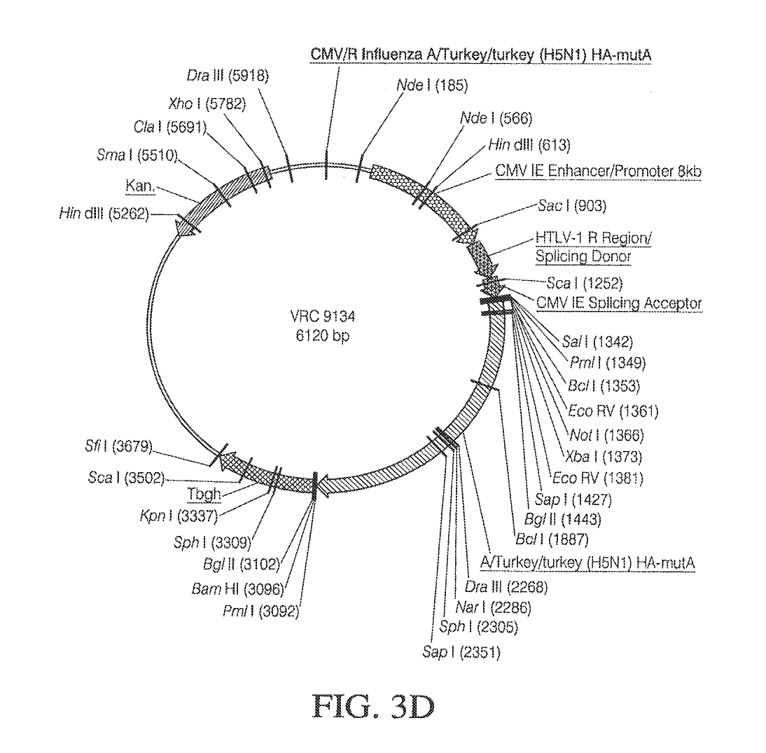

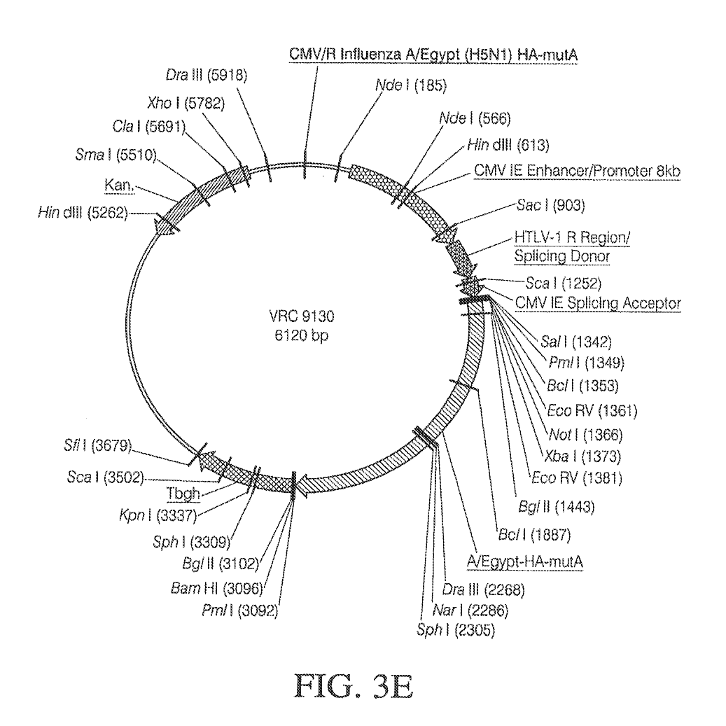

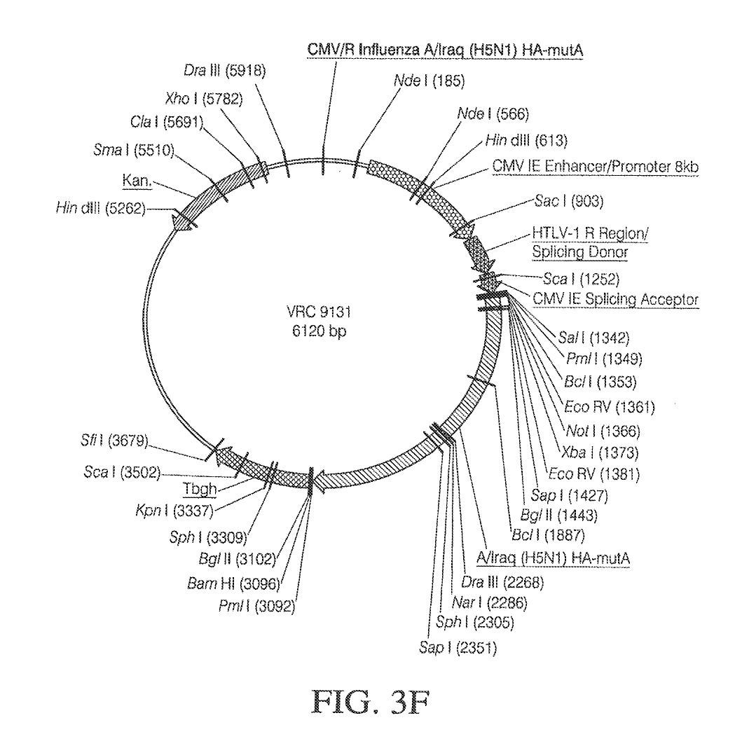

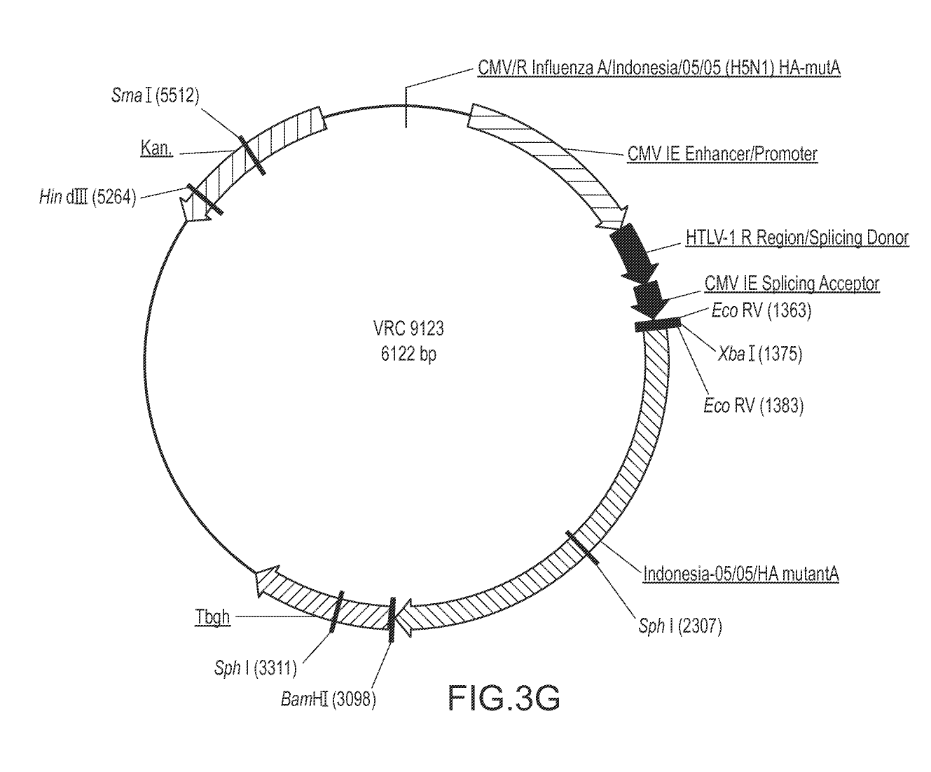

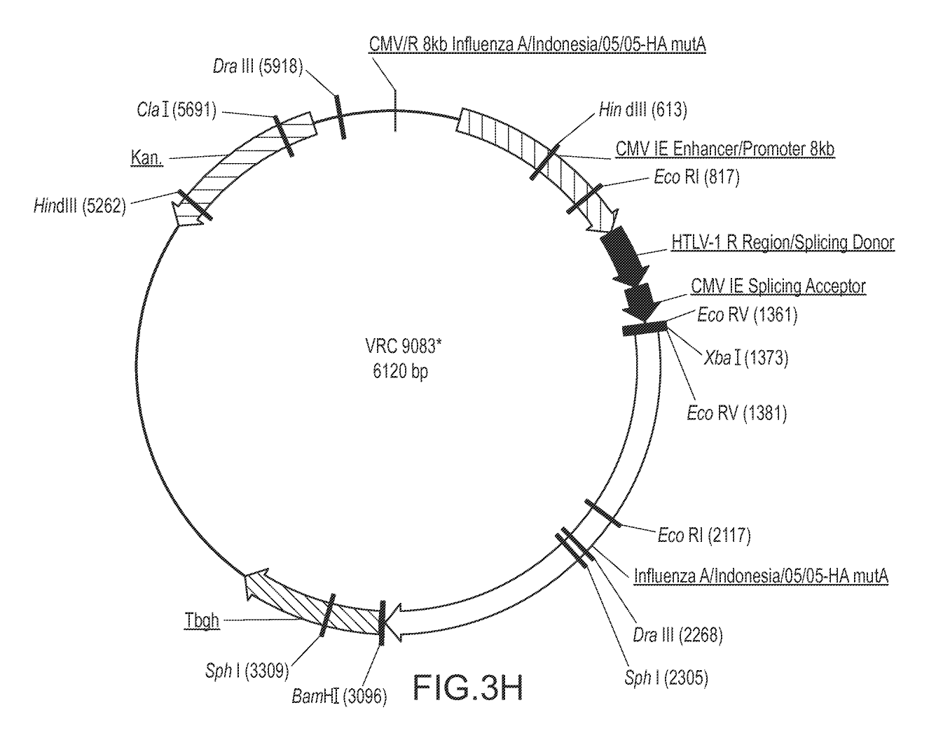

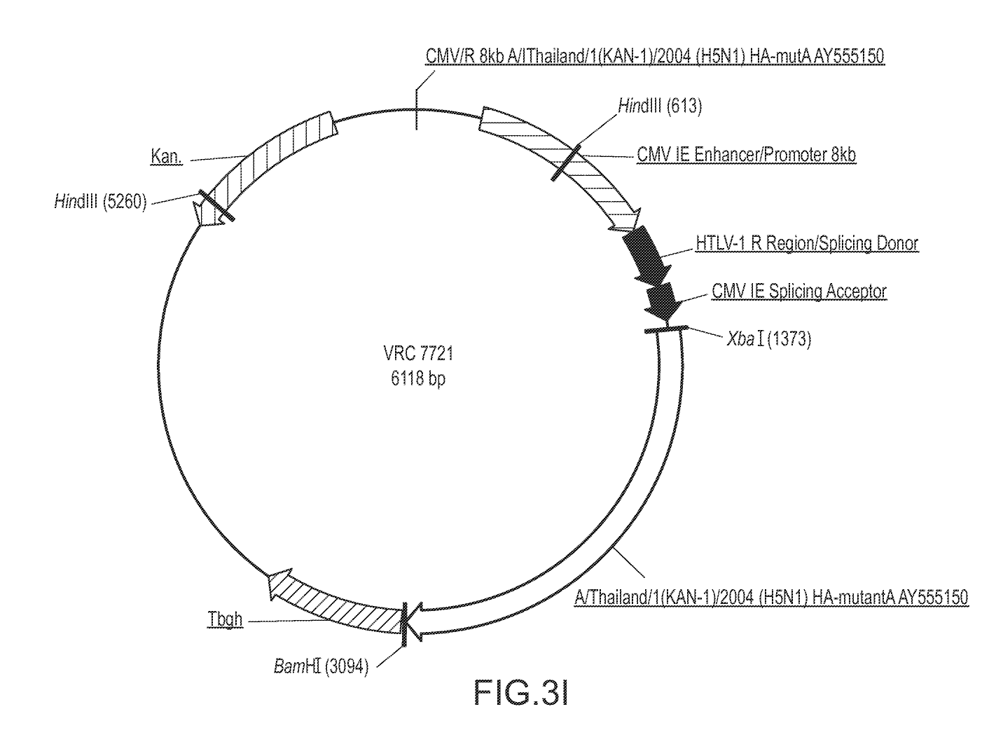

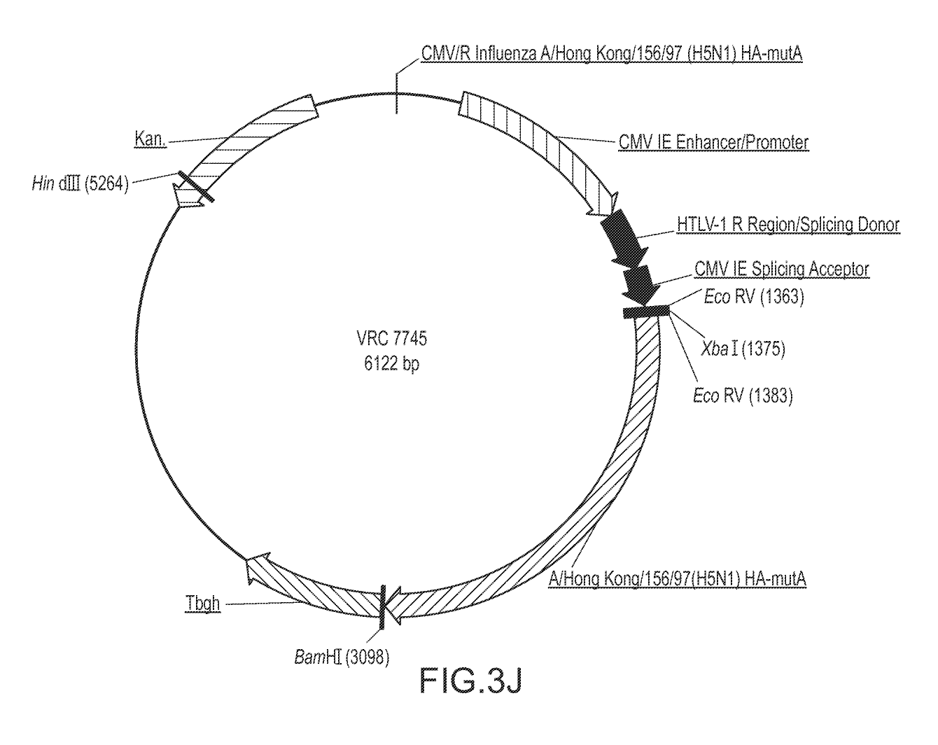

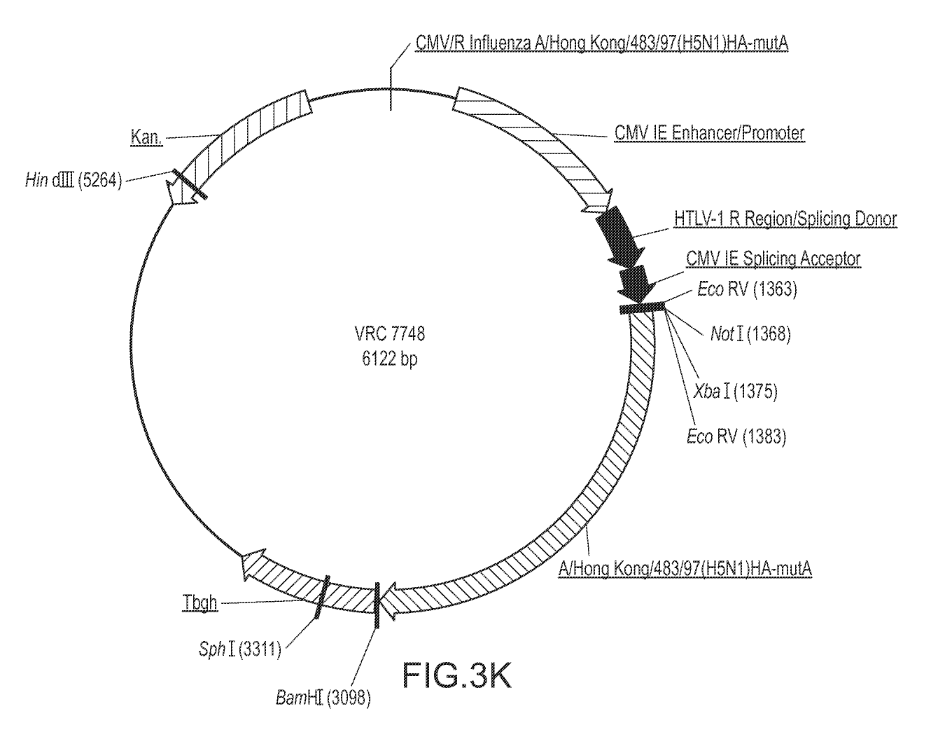

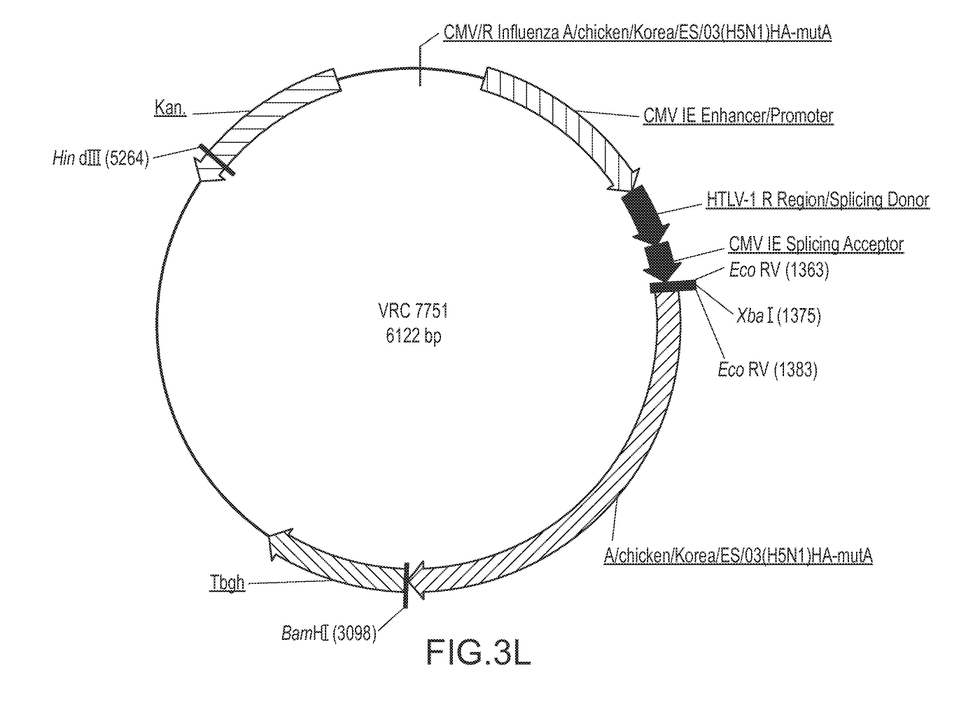

As an example, the DNA construct of the codon-optimized HA mutA of Influenza A/Anhui/1/2005(H5N1) in a CMV/R expression vector is presented in FIG. 3A. The nucleotide sequence of this DNA construct is presented as SEQ ID NO.: 14.

As an example, the DNA construct of the codon-optimized HA mutA of A/Vietnam/1203/2004(H5N1) in a CMV/R expression vector is presented in FIG. 3B. The nucleotide sequence of this DNA construct is presented as SEQ ID NO.: 15.

As an example, the DNA construct of the codon-optimized HA mutA of Influenza A/Nigeria/641/2006(H5N1) in a CMV/R expression vector is presented in FIG. 3C. The sequence of this DNA construct is presented as SEQ ID NO.: 16.

As an example, the DNA construct of the codon-optimized HA mutA of Influenza A/turkey/Turkey/1/2005(H5N1) in a CMV/R expression vector is presented in FIG. 3D. The sequence of this DNA construct is presented as SEQ ID NO.: 20.

As an example, the DNA construct of the codon-optimized HA mutA of Influenza A/Egypt/2782-NAMRU3/2006(H5N1) in a CMV/R expression vector is presented in FIG. 3E. The sequence of this DNA construct is presented as SEQ ID NO.: 21.

As an example, the DNA construct of the codon-optimized HA mutA of Influenza A/Iraq/207-NAMRU3/2006(H5N1) in a CMV/R expression vector is presented in FIG. 3F. The sequence of this DNA construct is presented as SEQ ID NO.: 22.

As an example, the DNA construct of the codon-optimized HA mutA of Influenza A/Indonesia/05/05 (H5N1) in a CMV/R expression vector is presented in FIG. 3G. The sequence of this DNA construct is presented as SEQ ID NO.: 23.

As an example, the DNA construct of the codon-optimized HA mutA of Influenza A/Indonesia/05/05 in a CMV/R 8κB expression vector is presented in FIG. 3H. The sequence of this DNA construct is presented as SEQ ID NO.: 24.

As an example, the DNA construct of the codon-optimized HA mutA of Influenza A/Thailand/1 (KAN-1)/2004 (H5N1) in a CMV/R 8κB expression vector is presented in FIG. 3I. The sequence of this DNA construct is presented as SEQ ID NO.: 25.

As an example, the DNA construct of the codon-optimized HA mutA of Influenza A/Hong Kong/156/97(H5N1) in a CMV/R expression vector is presented in FIG. 3J. The sequence of this DNA construct is presented as SEQ ID NO.: 26.

As an example, the DNA construct of the codon-optimized HA mutA of Influenza A/Hong Kong/483/97(H5N1) in a CMV/R expression vector is presented in FIG. 3K. The sequence of this DNA construct is presented as SEQ ID NO.: 27.

As an example, the DNA construct of the codon-optimized HA mutA of Influenza A/chicken/Korea/ES/03(H5N1) in a CMV/R expression vector is presented in FIG. 3L. The sequence of this DNA construct is presented as SEQ ID NO.: 28.

SEQ ID NOs: 14-16 and 20-28 were used in the examples, in addition to the 8κB versions of SEQ ID NOs: 26-28.

### Example 2

### Univalent HA DNA Vaccination and Response in Mice

Animals were immunized with each of the 10 different DNA constructs via IM route. 6-8 week old Female BALB/c mice were purchased from The Jackson Laboratory and maintained in the AAALAC accredited Vaccine Research Center Animal Care Facility (Bethesda, Md.) under pathogen-free conditions. All experiments were approved by the Vaccine Research Center Animal Care and Use Committee. The mice were immunized as described in Z.-Y. Yang et al., Nature 428, 561 (2004), herein incorporated by reference in its entirety.

Mice (10 animals for all test groups, 20 animals for the negative control group) were immunized three times with total 15 μg DNA construct in 100 μl of PBS (pH 7.4) intramuscularly at weeks 0, 3, 6. For the single DNA construct groups, the DNA construct in a volume of 100 μl was administered to each animal: pCMV/R 8κB, pCMV/R 8κB-HA(A/Indonesia/05/2005), pCMV/R-HA(A/Anhui/1/2005), pCMV/R 8κB-HA(A/Thailand/1(KAN-1)/2004), pCMV/R 8κB-HA(A/Hong Kong/156/1997), pCMV/R 8κB-HA(A/Hong Kong/483/1997), 8κB-pCMV/R HA(A/chicken/Korea/ES/2003), pCMV/R-HA(A/turkey/Turkey/1/2005), pCMV/R-HA(A/Egypt/2782-NAMRU3/2006), pCMV/R-HA(A/chicken/Nigeria/641/2006), and pCMV/R-HA(A/Iraq/207-NAMRU3/2006). The 10 DNA construct combination group (10 HA) received 1.5 μg DNA for each of the 10 HA DNA constructs (total 15 μg) as used in the single DNA construct groups mentioned above. For the two 5 DNA construct combination groups [5 HA (Set 1), 5 HA(Set 2)], 3 μg of each DNA construct were used as total DNA remained the same (15 μg). 5 HA (Set 1) group contained: pCMV/R 8κB-HA(A/Hong Kong/156/1997), pCMV/R 8κB-HA(A/chicken/Korea/ES/2003), pCMV/R HA(A/turkey/Turkey/1/2005), pCMV/R-HA(A/Egypt/2782-NAMRU3/2006), and pCMV/R-HA(A/chicken/Nigeria/641/2006). 5 HA (Set 2) group contained: pCMV/R 8κB-HA(A/Indonesia/05/2005), pCMV/R-HA(A/Anhui/1/2005), pCMV/R 8κB-HA(A/Thailand/1 (KAN-1)/2004), pCMV/R 8κB-HA(A/Hong Kong/483/1997), and pCMV/R-HA(A/Iraq/207-NAMRU3/2006).

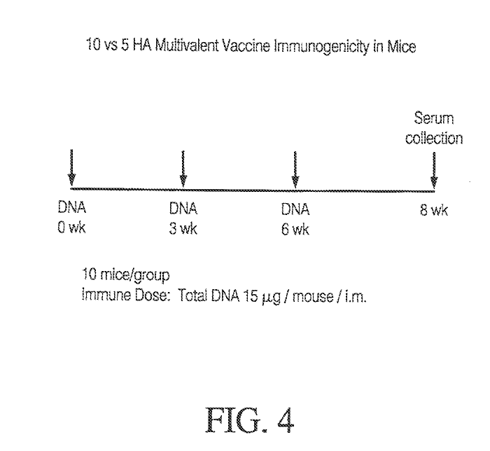

Serum was collected 10 days after the last vaccination. Pool serum from each group was collected 14 days after the third immunization. The immunization scheme is depicted in FIG. 4.

The recombinant lentiviral vectors expressing a luciferase reporter gene were produced as described in Kong, et al., Protective immunity to lethal challenge of the 1918 pandemic influenza virus by vaccination. Proc Natl Acad Sci USA 103: 15987-15991 (2006), herein incorporated by reference in its entirety. A 10 cm dish of 293T cells was cotransfected with 7 ng of pCMVRΔ8.2, 7 ng of pHR'CMV-Luc, 400 ng of CMV/R 8κB H5 HA (KAN1) and 100 ng of CMV/R N1 NA (KAN1) plasmids using ProFection Mammalian Transfection System (Promega, Madison, Wis.). Cells were transfected overnight, washed, and replenished with fresh medium. Forty-eight hours later, supernatants were collected and filtered through a 0.45 μm syringe filter. The filtered supernatants were aliquotted and used immediately or frozen at −80° C. For the neutralization assay, antisera from immunized animals were heat-inactivated at 55° C. for 30 minutes and mixed with 50 μl of pseudovirus at various dilutions. The sera/virus mixture was then added to 293A cells in 96-well B&W TC Isoplates (Wallac, Turku, Finland; 12,000 cells/well). The plates were washed and fresh medium was added 2 hours later. Cells were lysed in mammalian cell lysis buffer (Promega, Madison, Wis.) 24 hrs. after infection and luciferase activity was measured using Luciferase Assay System (Promega, Madison, Wis.).

The following strains were used for the production of pseudotyped viruses: for HA we used A/Thailand/1(KAN-1)/2004; A/Vietnam/1203/2004; A/Hong Kong/156/1997; A/Hong Kong/483/1997; A/chicken/Korea/ES/2003; A/Indonesia/05/2005; A/turkey/Turkey/1/2005; A/Egypt/2782-NAMRU3/2006; A/chicken/Nigeria/641/2006; A/Iraq/207-NAMRU3/2006; A/Anhui/1/2005, and for N1 NA we used A/Thailand/1(KAN-1)/2004.

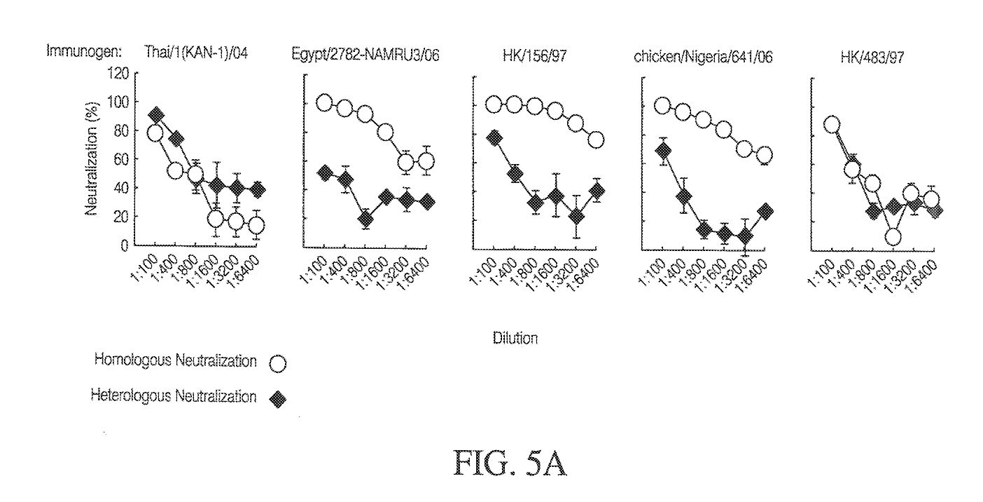

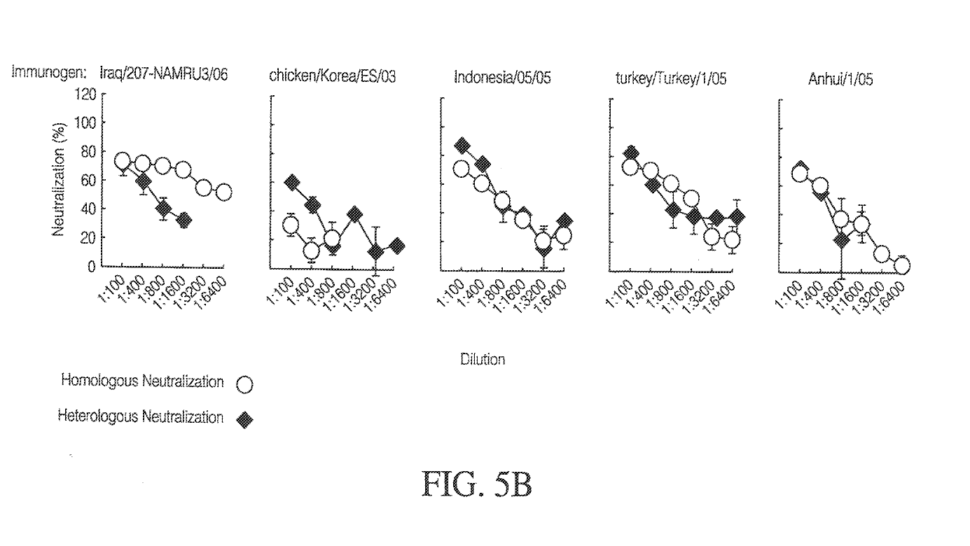

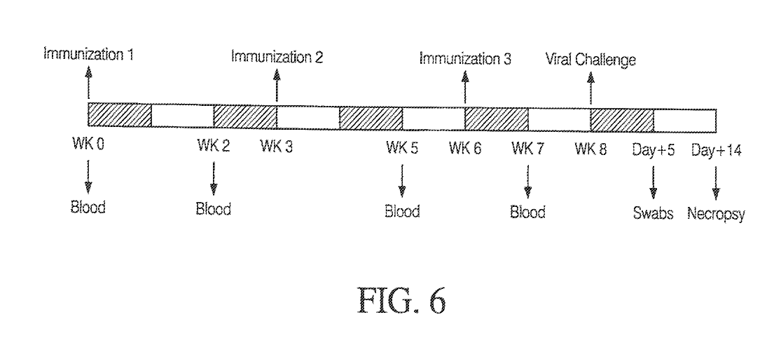

The serum collected from the mice were subject to neutralization of both homologous and heterologous (A/Vietnam/1203/04 as Vietnam 1203/04) H5 HA pseudotyped lentiviral vectors as indicated in FIG. 5. Serum from each group was serially diluted (1:100 to 1:6400). Error bars at each point indicate the standard deviation; each sample was evaluated in triplicate. Different degrees of neutralization among various H5 pseudoviruses were observed among different HA immunized mice. The serum antibody responses were evaluated 14 days after the third immunization, by its ability to neutralize the in house developed H5 HA pseudotyped lentiviral vectors. The pseudotyped lentiviral neutralization is a much more sensitive assay compared to microneutralization and HAI titers.

There were variable, but significant titers and cross reactivity to VN 1203/04 among the response to different immunogens. We noted marked titers with >90% neutralization even at dilutions of 1:6400 against homologous pseudotyped lentivirus for some immunogens (e.g., Nigeria, HK 456) whereas lesser immune response for others (e.g., Korea strain) [FIG. 5]. Heterologous neutralization to Vietnam 1203/04 was variable with significant neutralization at dilutions up to 1:400 [FIG. 5]. The ability of these immunogens to generate robust cross-reactive antibodies, particularly among phylogenetically closely related strains of influenza is consistent with previous observations.

### Example 3

### Multivalent HA Vaccination Response in Mice

In order to evaluate the ability of mice to generate a potent immune response, a combination of 10 immunogens given at a proportionally lower concentration (1.5 μg per immunogen) was administered intramuscularly to mice as described in Example 2. Similar to the univalent experimental schema, the mice were bled 14 days after the 3rd vaccination.

FIGS. 1A-C depict the potency of neutralization after 10 HAs multivalent vaccination in mice. Humoral immunity and potency of neutralization were evaluated after vaccination with DNA constructs expressing H5 HA protein, by HA pseudotyped lentiviral inhibition assay. The DNA vaccine consisted of 10 DNA constructs (1.5 μg each) expressing HA proteins from the following 10 different H5 strains indicated by asterisks in the figure: A/Thailand/1(KAN-1)/2004; A/Hong Kong/156/1997; A/Hong Kong/483/1997; A/chicken/Korea/ES/2003; A/Indonesia/05/2005; A/Turkey/Turkey/1/2005; A/Egypt/2782-NAMRU3/2006; A/chicken/Nigeria/641/2006; A/Iraq/207-NAMRU3/2006; A/Anhui/1/2005. The antisera were tested against 12 pseudoviruses as indicated in FIGS. 1A-C, including 10 homologous pseudoviruses and two non-immunized H5 strains (without asterisk in the key) in varying dilutions. Error bars at each point indicate the standard deviation; each sample was evaluated in triplicate. The 10 HA-immunized mice elicited different degrees of neutralization against homologous or heterologous H5 HA pseudoviruses. In general, the immunized serum can neutralize all tested pseudotyped lentiviruses in low dilutions, with some able to neutralize at significantly higher dilutions.

As shown in FIG. 1B, selection of the immunogen ‘combination’ is important for effective elicitation of a potent immune response measured by the pseudotype neutralization assay. Mice immunized with five different H5 HA DNAs elicited neutralization against only some H5 HA pseudotyped lentiviral vectors. Groups of mice (n=10) were immunized with 15 μg of DNA construct (3 μg each) three times at 3 week intervals. Serum pools from the immunized animals were collected 14 days after the third immunization. The DNA vaccine consisted of 5 DNA constructs expressing HA proteins from 5 different H5 strains: A/Hong Kong/156/1997 as HK156; A/chicken/Korea/ES/2003 as C/Korea; A/Turkey/Turkey/1/2005 as Turkey; A/Egypt/2782-NAMRU3/2006 as Egypt; A/chicken/Nigeria/641/2006 as Nigeria (indicated by asterisks). The serum was tested against the 5 homologous pseudoviruses as their HA was in the 5 DNA construct vaccine (asterisk) and seven non-immunized H5 strains (without an asterisk) in different dilutions. Error bars at each point indicate the standard deviation; each sample was evaluated in triplicate. The 5 HA-immunized mice elicited different degrees of neutralization against homologous or heterologous H5 HA pseudoviruses. Note that the responses are highly variable against the heterologous viruses while the homologous viral neutralizations were as predicted even in low dilutions, with A/Chicken/Korea/ES/2003 being the sole exception.

As depicted in FIG. 1C, mice were immunized with a multivalent (5) H5 HA DNA elicited neutralization against broadly divergent strains of H5 HA pseudotyped lentiviral vectors. The immunization schema and the DNA construct concentration were similar to that in FIG. 1B. The DNA vaccine consists of 5 DNA constructs (3 μg each) expressing HA proteins from 5 different H5 strains: A/Thailand/1(KAN-1)/2004 as KAN-1; A/Hong Kong/483/1997 as HK483; A/Indonesia/05/2005 as Indonesia; A/Iraq/207-NAMRU3/2006 as Iraq; A/Anhui/1/2005 as Anhui The serum was tested against the 5 homologous pseudoviruses (indicated by an asterisk) and seven non-immunized H5 strains (indicated without an asterisk) at different dilutions. Error bars at each point indicate the standard deviation; each sample was evaluated in triplicate. Mice elicited different degrees of neutralization against homologous or heterologous H5 HA pseudoviruses. The immunized mice were found to neutralize all test homologous or heterologous pseudotyped lentiviruses in low dilutions.

Remarkably, there were impressive titers and cross neutralization of the 10 immunogen multivalent vaccine with >80% neutralization against 6 out of 12 H5 HA pseudoviruses at dilutions of up to 1:400. Even at a log lower DNA concentration, the immune response was comparable to a 15 μg vaccine dose (FIG. 1A).

To evaluate the retention of the breadth of immune response with a lower number of immunogens, two different combinations of 5 immunogen multivalent vaccines were selected based on the phylogenetic diversity of HA among the avian influenza viruses and the degree of immune response each immunogen was capable of eliciting in the univalent vaccination schema as described in Example 2.

There is significant variability in the breadth of immune response (measured by the pseudotype neutralization assay as above) between the two sets of 5 immunogen multivalent vaccines (FIGS. 1B and 1C). In set 1, while the homologous pseudotyped lentivirus neutralization was comparable to the univalent and the 10 immunogen multivalent immune response, fewer cross-reactive antibodies were detected, directed most prominently against A/Iraq/207-NAMRU3/2006 and A/Egypt/2782-NAMRU3/2006 (FIG. 1B; 5 HA, Set 1) and there were robust cross reactive antibodies only against Iraq (FIG. 1B). There was minimal to highly variable immune response to the other heterologous pseudotyped lentivirus neutralization. In contrast, set 2 elicited consistent and comparable neutralization against both homologous and heterologous viruses at titers exceeding 1:400 against most of the tested HA strains (FIG. 1C; 5 HA, Set 2), as observed in the 10 component multivalent DNA vaccine. There was a consistent and comparable robust immune response against both homologous and heterologous viruses even at high dilutions (1:800) against some pseudoviruses (FIG. 1C), as observed in set 2.

It was therefore possible to use multivalent DNA immunization and selection based on neutralizing antibody breadth and phylogenetic relationships to reduce the number of components in the vaccine while maintaining substantial breadth of cross neutralization.

### Example 4

Protection of DNA-Vaccinated Mice Against Challenge with Heterologous H5N1 A/Vietnam/1203/2004 Influenza Virus

Sixty-eight weeks after the last immunization, female BALB/c mice were lightly anesthetized with Ketamine/Xylazine and inoculated intranasally with 10 LD₅₀ of A/Vietnam/1203/04 virus diluted in phosphate-buffered saline in a 50 μl volume. Mice were monitored daily for morbidity and measured for weight loss and mortality for 21 days post infection. Any mouse that had lost more than 25% of its body weight was euthanized. All experiments involving the HPAI virus were conducted in an AAALAC accredited facility (BioQual Inc., Gaithersburg, Md.) under BSL 3 conditions that included enhancements required by the USDA and the Select Agent Program.

The control animals, injected with the DNA construct with no insert, died within 10 days of infection. Complete survival was observed in the groups immunized with the 10 component and set 2 of the 5 component multivalent DNA vaccines (FIG. 11). Immunization with HA derived from the A/Indonesia/05/2005 strain or set 1 of the 5 component multivalent DNA vaccine showed survival approaching 90%. In contrast, animals injected with HA DNA construct derived from A/Anhui/1/2005, which has diverged more from A/Vietnam/1203/2004, showed a lower percent survival (70%) after lethal viral challenge.

Survival differences between groups were assessed using a log-rank test and the Gehan-Wilcoxon test on the survival curves for pairs of groups. A test was deemed significant if the p-value was <0.01. Mice injected IM with different HAs, A/Indonesia/5/05, A/Anhui/1/05, 10HA, 5 HA (Set 1), or 5 HA (Set 2) showed a significant difference compared to control (all p values <0.001). Among the HA-immunized groups, there was no significant difference between any two groups (p>0.08 for all comparisons). For example, no significant difference was observed between the A/Anhui/1/05 group, which had the least survival among the HA immunized groups (7 out of 10), and other HA groups: A/Indonesia/5/05 (p=0.377), 10 HA (p=0.082), 5 HA (Set 1) (p=0.101), or 5 HA (Set 2) (p=0.411). Therefore, we cannot exclude the possibility that the 3 deaths in the A/Anhui/1/05 group may have been due to random chance.

### Example 5

### Optimization of Non-Needle (Agro-Jet®) DNA Immunization in Chickens

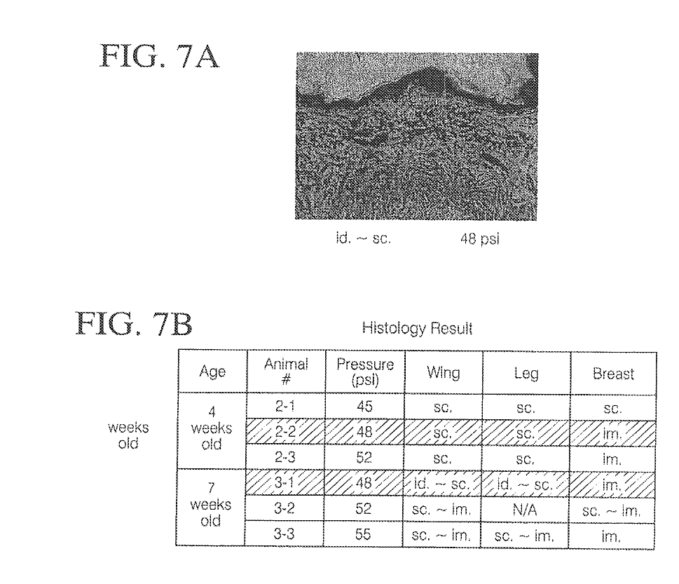

Chickens (4 or 7 weeks old) were injected with India ink in PBS as a probe to determine the best condition to deliver vaccines to chickens. Wing, leg and the breast of the chickens were subjected to different pressure (in psi) settings of the Agro-Jet® device. Shortly after the injections of the India ink solution, tissue autopsy samples from the injected sites were collected and examined under the microscope to determine the location of the India ink probe. Leg skin histology sections indicate that the best pressure for delivery of the vaccine via Agro-Jet® is 48 psi as shown in FIG. 7A for intra-dermal/sub-cutaneous (ID/SC) delivery. Other pressure settings resulted in sub-cutaneous (SC), intra-muscular (IM), or sub-cutaneous/intra-muscular (SC/IM) delivery, as shown in FIG. 7B. Highlighted rows of immunization conditions were selected for use.

The injection conditions were determined by histologic analysis of tissues that received injections of India ink; a pressure of 48 psi was chosen since it provided consistent delivery into intradermal and subcutaneous tissues (Table 4).

### Example 6

### Neutralizing Antibody Responses in Chickens after HA DNA Immunization

Since it is desirable to confer protective immunity in poultry and HA DNA vaccination was effective in mice, we next examined the breadth and potency of single or multiple HA DNA construct immunization in chickens.

The ability of chickens to generate specific antibodies was assessed with three strains that showed broad cross protection in mouse studies (A/Vietnam/1203/2004, A/Anhui/1/2005 and A/Indonesia/05/2005), administered individually or in combination, by different injection methods. In addition to needle injection, a needle-free repetitive injection device, Agro-Jet® (Medical International Technology, Inc., Denver, Colo.), was analyzed. This device disperses the 0.1 to 5 ml injection doses into the dermal, subcutaneous, or intramuscular tissue depending upon the pressure adjustments, powered by a CO₂ gas pressure plunger. Higginson R, et al. (2005) Br J Nurs 14: 632. The injection conditions were determined by histologic analysis of tissues that received injections of India ink; a pressure of 48 psi was chosen since it enabled consistent delivery into intradermal and subcutaneous tissues (FIG. 9).

Immunization of chickens with the control plasmid (CMV/R) without an HA gene insert elicited minimal neutralizing antibody titers compared to HA immunized animals 1 week after 3 DNA immunizations. Birds immunized with either monovalent or multivalent HA DNA vaccines generated significant neutralization titers (FIG. 10 and Table 3). In general, there was a progressive increase in the amount of neutralization after each successive DNA vaccination with maximal response at 1 week after the 3^{rd} DNA immunization, with highest and most consistent levels in the trivalent vaccine group delivered with the AGRO-JET® device.

Neutralization of Indonesia HA strain was the most robust, with neutralization nearing 100% at titers greater than 1:3200. Both the monovalent and multivalent vaccines elicited robust homologous (FIG. 10, A/Indonesia/05/2005) and heterologous HA neutralization (FIG. 10, A/Nigeria/641/05). Similar responses were noted in the other groups, including administration of monovalent HA-A/Indonesia/05/2005 subcutaneously by needle syringe (FIG. 10) and via AGRO-JET® (FIG. 10).

TABLE 3

| | | | | | | LAI Titer | |
|---|---|---|---|---|---|---|---|
| | Animal | HI Titer | | NT Titer | | (VN1203) | |
| Immunogen | ID | Week 5 | Week 7 | Week 5 | Week 7 | Week 5 | Week 7 |
| Agro-Jet x3 | 202 | 40 | 40 | 10 | 10 | <100 | <100 |
| Control | 203 | 40 | 40 | 10 | 10 | <100 | <100 |
| 500 μg | 204 | 20 | 20 | 10 | 10 | <100 | <100 |
| | 205 | 40 | 20 | 10 | 10 | <100 | <100 |
| | 206 | 40 | 20 | 10 | 10 | <100 | <100 |
| | 207 | 20 | 20 | 10 | 10 | <100 | <100 |
| | 208 | 40 | 40 | 10 | 10 | <100 | <100 |
| | 209 | 40 | 20 | 10 | 10 | <100 | <100 |
| Agro-Jet x3 | 211 | 80 | 80 | 160 | 160 | 141 | 884 |
| A/Indonesia/05/05 | 212 | 20 | 640 | 160 | 640 | 156 | >3200 |
| 500 μg | 213 | 40 | 80 | 40 | 160 | 452 | 806 |
| | 214 | 20 | 160 | 20 | 160 | 396 | 670 |
| | 215 | 80 | 40 | 40 | 320 | 696 | 1436 |
| | 216 | 40 | 80 | 80 | 320 | 1284 | >3200 |
| | 217 | 40 | 40 | 20 | 640 | 216 | >3200 |
| | 218 | 20 | 40 | 40 | 160 | 159 | 1088 |
| Agro-Jet x3 | 233 | 40 | 640 | 40 | 640 | 381 | 1836 |
| A/Indonesia/05/05 | 234 | 40 | 1280 | 40 | 1280 | 102 | 2911 |
| A/Vietnam/1203/04 | 235 | 20 | 1280 | 40 | 320 | <100 | >3200 |
| A/Anhui/01/05 | 236 | 40 | 1280 | 20 | 640 | 118 | 3013 |
| 500 μg | 237 | 20 | 1280 | 40 | 1280 | 237 | >3200 |
| | 238 | 20 | 160 | 10 | 10 | <100 | <100 |
| | 239 | 20 | 1280 | 20 | 640 | 145 | >3200 |
| | 240 | 80 | 1280 | 40 | 640 | 127 | 2780 |
| Agro-Jet x2 | 242 | 40 | 40 | 40 | 10 | <100 | <100 |
| A/Indoensia/05/05 | 243 | 40 | 80 | 40 | 10 | <100 | <100 |
| 500 μg | 244 | 40 | 80 | 40 | 10 | <100 | <100 |
| | 245 | 40 | 1280 | 20 | 320 | 100 | 992 |
| | 246 | 40 | 1280 | 40 | 320 | 190 | 2618 |
| | 247 | 40 | 1280 | 40 | 1280 | <100 | 2679 |
| | 248 | 40 | 10 | 40 | 20 | <100 | <100 |
| | 249 | 80 | 640 | 80 | 640 | 183 | 2127 |
| sc N/S x3 | 251 | 80 | 20 | 20 | 40 | 180 | 161 |
| A/Indonesia/05/05 | 252 | 80 | 80 | 320 | 320 | 531 | 764 |
| 500 μg | 253 | 80 | 80 | 40 | 160 | 213 | 857 |
| | 254 | 40 | 160 | 1280 | 80 | 2233 | 985 |
| | 255 | 320 | 320 | 1280 | 640 | 2564 | 2878 |
| | 256 | 40 | 80 | 10 | 10 | 163 | 104 |
| | 257 | 80 | 320 | 80 | 80 | 493 | 476 |
| | 258 | 80 | 80 | 320 | 160 | 1269 | 650 |
| im N/S x3 | 259 | 40 | 80 | 1280 | 80 | 971 | 583 |
| A/Indonesia/05/05 | 260 | 160 | 160 | 320 | 160 | 1521 | 864 |
| 500 μg | 261 | 160 | 160 | 640 | 80 | 1945 | 532 |
| | 262 | 40 | 80 | 40 | 40 | 197 | 254 |
| | 263 | 80 | 40 | 40 | 20 | 631 | 291 |
| | 264 | 320 | 1280 | 640 | 160 | 2366 | 1688 |
| | 265 | 80 | 1280 | 1280 | 320 | 2132 | 1539 |
| | 266 | 40 | 80 | 80 | 80 | 416 | 394 |

### Example 7

### Antibody Response in Chickens After Univalent and Multivalent HA DNA Immunization

Initially, the ability of chickens to generate specific antibodies measurable by HAI and our pseudotype neutralization assays, after immunization with our HA DNA univalent and multivalent vaccines (A/Vietnam/1203/2004, A/Anhui/1/2005 and A/Indonesia/05/2005) using multiple routes and methods of immunization (Table 4A) was evaluated.

The study was carried out in the AAALAC-accredited animal facility at the University of Maryland School of Medicine. Six groups of 8 one-day old male and female SPAFAS White Leghorn Chickens, Gallus domesticus, were obtained from Charles River Laboratories (Connecticut). The animals were housed in brooder and grower cages (McMurray Hatcheries, Iowa). Feed (Teklad Japanese Quail Diet—3050, Harlan-Teklad, WI) and water were provided to the animals ad libitum. The study was performed in strict accordance with the “Guide” after approvals from the Animal Care and Use Committees of the Vaccine Research Center, NIH and the University of Maryland. DNA immunizations were performed at 0, 3 and 6 weeks. The DNA vaccines used in the study include three DNA constructs that encode for the HA protein of the Anhui, Vietnam and Indonesian subtypes of the H5N1 virus. A total dose of 500 μg of one or a combination of the following DNA constructs in a volume of 250 μl was administered to each animal: pCMV/R, pCMV/R-HA(A/Indonesia/05/2005), pCMV/R-HA(A/Anhui/1/2005), and pCMV/R-HA(A/Vietnam/1203/2004). Groups 1-4 received intradermal/subcutaneous immunizations via Agro-Jet® with pCMV/R, with pCMV/R-HA(A/Indonesia/05/2005), with pCMV/R-HA(A/Indonesia/05/2005) plus pCMV/R-HA(A/Anhui/1/2005) plus pCMV/R-HA(A/Vietnam/1203/2004), or with pCMV/R-HA(A/Indonesia/05/2005) respectively; Group 5 received subcutaneous immunizations via needle and syringe with pCMV/R-HA(A/Indonesia/05/2005); and Group 6 received intramuscular immunizations via needle and syringe with pCMV/R-HA(A/Indonesia/05/2005). Blood samples were collected from the alar vein of the birds at various intervals. All groups were challenged at week 8 via nares, mouth, and eyes with 5×10⁶ EID 50 /ml of A/Vietnam 1203/4 H5N1 viruses. For the viral load study, cloacal and tracheal swabs were collected from each animal on days 3 and 5 post challenge and titrated for virus infectivity in embryonated eggs. Chickens were monitored each day for signs of disease or death. Surviving chickens underwent necropsy on day 14 post challenge.

For dose response experiments, five groups were immunized with the trivalent HA vaccine (pCMV/R-HA(A/Anhui/1/2005), pCMV/R-HA(A/Indonesia/05/2005), and pCMV/R-HA(A/chicken/Nigeria/641/2006) using 500 μg (167 μg of each of the three DNA constructs), 50 μg (17 μg of each DNA construct), 5 μg (1.7 μg of each DNA construct), 0.5 μg (0.17 μg of each DNA construct) and a 500 μg control vector administered IM with needle and syringe, and an additional five groups were injected with the same DNA construct doses using the Agro-Jet® injection device.

For the microneutralization assays, neutralizing antibodies were titrated from serum samples collected week 5 and 7 post-vaccination and day 14 post-challenge. The microneutralization assay was performed using a 96-well plate format. Serum was treated with receptor-destroying enzyme (Denka Seiken Co.) and treated at 37° C. per the manufacturer's instructions. After an overnight incubation and subsequent inactivation samples were brought to a final dilution of 1:10 using PBS and each sample was serially diluted and virus, diluted to 100 TCID₅₀, was added to each well. The plates were then incubated at 37° C., 5% CO₂ for 1-2 hours. Following incubation, supernatants were used to infect a second 96-well plate of MDCK cells. Microplates were incubated at 4° C. for 15 minutes and then 37° C., 5% CO₂ for 45 minutes. Supernatants of serum and virus were then discarded and 200 μl of OptiMEM (containing 1× antibiotics/antimycotics, 1 μg/ml TPCK-trypsin) was added and incubated at 37° C., 5% CO₂ for 3 days. After 3 days, 50 μl of the supernatant from each well was transferred into a new 96-well microplate, and an HA assay was performed to calculate the antibody titers. Virus and cell controls were included in the assay.

Two-fold dilutions of heat-inactivated sera were tested in a microneutralization assay as previously described (Hoffman, et al. (2002) Vaccine 20:3165-3170) for the presence of antibodies that neutralized the infectivity of 100 TCID₅₀ (50% tissue culture infectious dose) of the A/Vietnam/1203/2004 H5N1 virus on MDCK cell monolayers by using two wells per dilution on a 96-well plate.

For Hemagglutination (HA) and hemagglutination inhibition (HAI) assays, the HA/HI titers were determined as described in Hoffmann et al., Vaccine 20: 3165-3170 (2002), herein incorporated by reference in its entirety. HA titers were calculated using 50 μl of 0.5% chicken red blood cell suspension in PBS added to 50 μl of two-fold dilutions of virus in PBS. This mix was incubated at room temperature for 30 minutes. The HA titers were calculated as the reciprocal value of the highest dilution that caused complete hemagglutination. HI titers were calculated by titrating 50 μl of receptor-destroying enzyme treated with antiserum and an equivalent amount of A/Vietnam/1203/2004 virus (four hemagglutinating doses) was added to each well. Wells were incubated at room temperature for 30 minutes and 50 μl of a 0.5% suspension of chicken red blood cells was added. HI titers were calculated after 30 minutes as the reciprocal of the serum dilution that inhibited hemagglutination.

In order to evaluate the method of delivery (route of immunization), frequency (time of immunization) and to compare univalent against multivalent vaccine strategies, six groups (Group 1 to 6) of eight animals immunized with different immunogen DNA and conditions as indicated in Table 4A. The multivalent vaccine received a third of the total dose for each of the components. Sera collected from the groups at different time points as indicated were subjected to hemagglutination inhibition (HI) assay. Geometric mean reciprocal end point titers (GMT) of the HI assay and Mean Death Time (MDT) with death value of the groups were calculated. Intra-muscular immunization (IM) by needle and syringe with different doses of total DNA as indicated in Table 4B were compared with Agro-Jet® intra-dermal/sub-cutaneous as Agro-Jet® (ID/SQ) as indicated in Table 4C. Total DNA ranging from 500 μg to 0.5 μg of the trivalent HA was administered to the animals as indicated in Tables 3A-C. Ten groups (Group 11 to 15 for (IM), Group 21 to 25 for (ID/SQ)) of eight animals immunized in different immunogen DNA and conditions as indicated in Tables 3A-C. Sera collected from the groups at different time points as indicated were subjected to hemagglutination inhibition (HI) assay. Geometric mean reciprocal end-point titers (GMT) of the HI assay and Mean Death Time (MDT) with death value of the groups were calculated. Detailed results of the assay with other assays from each individual animal are shown in Tables 4, 5, and 6.

TABLE 4A
Protective efficacy of different vaccines in various routes against highly pathogenic
A/VN/1203/04 (H5N1) challenge in chickens

| | | | | | HI | HI | | HI |
|---|---|---|---|---|---|---|---|---|
| | | | | | positive/ | positive/ | | positive/ |
| | | | | | total | total | | total |
| | | | | | animals | animals | Deaths/total | animals |
| | | | | | (GMT) 5 | (GMT) 7 | animals | (GMT) 2 |
| | Immunogen DNA | Amount | Route of | Time of | weeks | weeks | (MDT in | weeks |
| Group | in CMV/R vector | of DNA | immunization | Immunization | p.v. | p.v. | days) | p.c. |
| 1 | Control vector | 500 μg | Agro-jet | 3 (0, 3, 6 wks) | 0/8 | 0/8 | 8/8 (2) | 0 (all dead) |
| | | | (ID/SQ) | | | | | |
| 2 | HA (IN/05/05) | 500 μg | Agro-jet | 3 (0, 3, 6 wks) | 8/8 (70) | 8/8 (320) | 0/8 | 8/8 (285) |
| | | | (ID/SQ) | | | | | |
| 3 | HA (IN/05/05) | 500 μg | Agro-jet | 3 (0, 3, 6 wks) | 7/8 (34) | 7/8 (7/7) | 0/8 | 8/8 (303.7) |
| | HA (Anhui/01/05) | | (ID/SQ) | | | | | |
| | HA (VN/1203/04) | | | | | | | |
| 4 | HA (IN/05/05) | 500 μg | Agro-jet | 2 (0, 6 wks) | 8/8 (42.5) | 5/8 (516) | 0/8 | 8/8 (532.5) |
| | | | (ID/SQ) | | | | | |
| 5 | HA (IN/05/05) | 500 μg | Needle & | 3 (0, 3, 6 wks) | 8/8 (419) | 7/8 (211) | 0/8 | 8/8 (155) |
| | | | Syringe (SC) | | | | | |
| 6 | HA (IN/05/05) | 500 μg | Needle & | 3 (0, 3, 6 wks) | 8/8 (540) | 8/8 (117.5) | 0/8 | 8/8 (495) |
| | | | Syringe (IM) | | | | | |

p.v. = post vaccination,
p.c. = post challenge,
GMT = geometric mean reciprocal end point titer

TABLE 4B
Protective efficacy of different vaccines in various routes against highly pathogenic
A/VN/1203/04 (H5N1) challenge in chickens

| | | | | | | | | |
|---|---|---|---|---|---|---|---|---|
| | | | | | HI | HI | | HI |
| | | | | | positive/ | positive/ | | positive/ |
| | | | | | total | total | | total |
| | | | | | animals | animals | Deaths/total | animals |
| | | | | | (GMT) 5 | (GMT) 7 | animals | (GMT) 2 |
| | Immunogen DNA in | Amount | Route of | Time of | weeks | weeks | (MDT in | weeks |
| Group | CMV/R vector | of DNA | immunization | Immunization | p.v. | p.v. | days) | p.c. |

| | | | | | | |
|---|---|---|---|---|---|---|
| 11 | Control vector | 500 μg | Needle & | 2 (0, 3 wks) | 7/7 (2.4) | 0 (all dead) |
| | | | Syringe (IM) | | | |
| 12 | HA (IN/05/05) | 500 μg | Needle & | 2 (0, 3 wks) | 0/8 | 8/8 (325) |
| | HA (Anhui/01/05) | | Syringe (IM) | | | |
| | HA (Nigeria/641/05) | | | | | |
| 13 | HA (IN/05/05) | 50 μg | Needle & | 2 (0, 3 wks) | 0/8 | 8/8 (120) |
| | HA (Anhui/01/05) | | Syringe (IM) | | | |
| | HA (Nigeria/641/05) | | | | | |
| 14 | HA (IN/05/05) | 5 μg | Needle & | 2 (0, 3 wks) | 2/8 (4) | 6/8 (197) |
| | HA (Anhui/01/05) | | Syringe (IM) | | | |
| | HA (Nigeria/641/05) | | | | | |
| 15 | HA (IN/05/05) | 0.5 μg | Needle & | 2 (0, 3 wks) | 6/8 (3.8)* | 2/8 (200) |
| | HA (Anhui/01/05) | | Syringe (IM) | | | |
| | HA (Nigeria/641/05) | | | | | |

p.v. = post vaccination,
p.c. = post challenge,
GMT = geometric mean reciprocal end point titer
*5/6 dead by day 4 p.c., 1 died day 7 p.c.

TABLE 4C
Protective efficacy of different vaccines in various routes against highly pathogenic
A/VN/1203/04 (H5N1) challenge in chickens

| | | | | | | | | |
|---|---|---|---|---|---|---|---|---|
| | | | | | HI | HI | | HI |
| | | | | | positive/ | positive/ | | positive/ |
| | | | | | total | total | | total |
| | | | | | animals | animals | Deaths/total | animals |
| | | | | | (GMT) 5 | (GMT) 7 | animals | (GMT) 2 |
| | Immunogen DNA in | Amount | Route of | Time of | weeks | weeks | (MDT in | weeks |
| Group | CMV/R vector | of DNA | immunization | Immunization | p.v. | p.v. | days) | p.c. |

| | | | | | | |
|---|---|---|---|---|---|---|
| 21 | Control vector | 500 μg | Agro-jet | 2 (0, 3 wks) | 7/8 (5.1) | 1/8 (80) |
| | | | (ID/SQ) | | | |
| 22 | HA (IN/05/05) | 500 μg | Agro-jet | 2 (0, 3 wks) | 0/8 | 8/8 (580) |
| | HA (Anhui/01/05) | | (ID/SQ) | | | |
| | HA (Nigeria/641/05) | | | | | |
| 23 | HA (IN/05/05) | 50 μg | Agro-jet | 2 (0, 3 wks) | 0/8 | 8/8 (430) |
| | HA (Anhui/01/05) | | (ID/SQ) | | | |
| | HA (Nigeria/641/05) | | | | | |
| 24 | HA (IN/05/05) | 5 μg | Agro-jet | 2 (0, 3 wks) | 0/8 | 8/8 (182.5) |
| | HA (Anhui/01/05) | | (ID/SQ) | | | |
| | HA (Nigeria/641/05) | | | | | |
| 25 | HA (IN/05/05) | 0.5 μg | Agro-jet | 2 (0, 3 wks) | 6/8 (4.8)* | 2/8 (200) |
| | HA (Anhui/01/05) | | (ID/SQ) | | | |
| | HA (Nigeria/641/05) | | | | | |

p.v. = post vaccination,
p.c. = post challenge,
GMT = geometric mean reciprocal end point titer
*one chicken euthanized day 10 p.c. due to severe neurological problems. All others died by day 6 p.c.

TABLE 5

| | | | | | | | Pseudovirus | |
|---|---|---|---|---|---|---|---|---|
| | | | | | Serum | | (VN1203) | |
| | | | Serum | | NT Titer | | Neutralization | |
| | | Animal | HI Titer | | (MicroNeut) | | (IC50) | |
| Group | | ID | Week 5 | Week 7 | Week 5 | Week 7 | Week 5 | Week 7 |
| 1 | 500 g | 202 | 40 | 40 | 10 | 10 | <100 | <100 |
| | Control vector | 203 | 40 | 40 | 10 | 10 | <100 | <100 |
| | Agro-jet (ID/SQ) | 204 | 20 | 20 | 10 | 10 | <100 | <100 |
| | | 205 | 40 | 20 | 10 | 10 | <100 | <100 |
| | | 206 | 40 | 20 | 10 | 10 | <100 | <100 |
| | | 207 | 20 | 20 | 10 | 10 | <100 | <100 |
| | | 208 | 40 | 40 | 10 | 10 | <100 | <100 |
| | | 209 | 40 | 20 | 10 | 10 | <100 | <100 |
| 2 | 500 g | 211 | 80 | 80 | 160 | 160 | 141 | 884 |
| | HA (IN/05/05) | 212 | 20 | 640 | 160 | 640 | 156 | >3200 |
| | Agro-jet (ID/SQ) | 213 | 40 | 80 | 40 | 160 | 452 | 806 |
| | | 214 | 20 | 160 | 20 | 160 | 396 | 670 |
| | | 215 | 80 | 40 | 40 | 320 | 696 | 1436 |
| | | 216 | 40 | 80 | 80 | 320 | 1284 | >3200 |
| | | 217 | 40 | 40 | 20 | 640 | 216 | >3200 |
| | | 218 | 20 | 40 | 40 | 160 | 159 | 1088 |
| 3 | 500 g | 233 | 40 | 640 | 40 | 640 | 381 | 1836 |
| | HA (IN/05/05) | 234 | 40 | 1280 | 40 | 1280 | 102 | 2911 |
| | HA (Anhui/01/05) | 235 | 20 | 1280 | 40 | 320 | <100 | >3200 |
| | HA (VN/1203/04) | 236 | 40 | 1280 | 20 | 640 | 118 | 3013 |
| | Agro-jet (ID/SQ) | 237 | 20 | 1280 | 40 | 1280 | 237 | >3200 |
| | | 238 | 20 | 160 | 10 | 10 | <100 | <100 |
| | | 239 | 20 | 1280 | 20 | 640 | 145 | >3200 |
| | | 240 | 80 | 1280 | 40 | 640 | 127 | 2780 |
| 4 | 500 g | 242 | 40 | 40 | 40 | 10 | <100 | <100 |
| | HA (IN/05/05) | 243 | 40 | 80 | 40 | 10 | <100 | <100 |
| | Agro-jet (ID/SQ) | 244 | 40 | 80 | 40 | 10 | <100 | <100 |
| | 2X Immunizations | 245 | 40 | 1280 | 20 | 320 | 100 | 992 |
| | | 246 | 40 | 1280 | 40 | 320 | 190 | 2618 |
| | | 247 | 40 | 1280 | 40 | 1280 | <100 | 2679 |
| | | 248 | 40 | 10 | 40 | 20 | <100 | <100 |
| | | 249 | 80 | 640 | 80 | 640 | 183 | 2127 |
| 5 | 500 g | 251 | 80 | 20 | 20 | 40 | 180 | 161 |
| | HA (IN/05/05) | 252 | 80 | 80 | 320 | 320 | 531 | 764 |
| | Needle & Syringe (SC) | 253 | 80 | 80 | 40 | 160 | 213 | 857 |
| | | 254 | 40 | 160 | 1280 | 80 | 2233 | 985 |
| | | 255 | 320 | 320 | 1280 | 640 | 2564 | 2878 |
| | | 256 | 40 | 80 | 10 | 10 | 163 | 104 |
| | | 257 | 80 | 320 | 80 | 80 | 493 | 476 |
| | | 258 | 80 | 80 | 320 | 160 | 1269 | 650 |
| 6 | 500 g | 259 | 40 | 80 | 1280 | 80 | 971 | 583 |
| | HA (IN/05/05) | 260 | 160 | 160 | 320 | 160 | 1521 | 864 |
| | Needle & Syringe (IM) | 261 | 160 | 160 | 640 | 80 | 1945 | 532 |
| | | 262 | 40 | 80 | 40 | 40 | 197 | 254 |
| | | 263 | 80 | 40 | 40 | 20 | 631 | 291 |
| | | 264 | 320 | 1280 | 640 | 160 | 2366 | 1688 |
| | | 265 | 80 | 1280 | 1280 | 320 | 2132 | 1539 |
| | | 266 | 40 | 80 | 80 | 80 | 416 | 394 |

TABLE 6

| | | | | | | Pseudovirus | |
|---|---|---|---|---|---|---|---|
| | | | | | | Neutralization | |
| | | | | Serum | | IC50) | |
| | | Serum | | NT Titer | | VN/1203 | |
| | Animal | HI Titer | | (MicroNeut) | | IN/05/05 | |
| Group | ID | Week 5 | Week 7 | Week 5 | Week 7 | Week 5 | Week 5 |

| | | | | |
|---|---|---|---|---|
| 11 | 500 μg | 27 | 102 | 109 |
| | Control vector | 28 | <100 | <100 |
| | Needle & Syringe (IM) | 29 | <100 | <100 |
| | | 268 | <100 | <100 |
| | | 269 | 150 | 134 |
| | | 270 | <100 | <100 |
| | | 271 | <100 | <100 |
| 12 | 500 μg | 30 | 125 | 167 |
| | HA (IN/05/05) | 31 | <100 | 115 |
| | HA (Anhui/01/05) | 32 | <100 | 142 |
| | HA (Nigeria/641/05) | 33 | <100 | <100 |
| | Needle & Syringe (IM) | 272 | 169 | 181 |
| | | 273 | <100 | 142 |
| | | 274 | <100 | <100 |
| | | 275 | 194 | 614 |
| 13 | 50 μg | 34 | <100 | 131 |
| | HA (IN/05/05) | 35 | <100 | 145 |
| | HA (Anhui/01/05) | 36 | <100 | <100 |
| | HA (Nigeria/641/05) | 37 | <100 | <100 |
| | Needle & Syringe (IM) | 277 | <100 | <100 |
| | | 278 | <100 | 140 |
| | | 279 | <100 | <100 |
| | | 280 | <100 | <100 |
| 14 | 5 μg | 38 | 125 | 149 |
| | HA (IN/05/05) | 39 | <100 | <100 |
| | HA (Anhui/01/05) | 40 | <100 | <100 |
| | HA (Nigeria/641/05) | 41 | <100 | <100 |
| | Needle & Syringe (IM) | 281 | 144 | 111 |
| | | 282 | <100 | <100 |
| | | 283 | <100 | <100 |
| | | 284 | <100 | <100 |
| 15 | 0.5 μg | 43 | <100 | <100 |
| | HA (IN/05/05) | 44 | <100 | <100 |
| | HA (Anhui/01/05) | 50 | <100 | <100 |
| | HA (Nigeria/641/05) | 285 | <100 | <100 |
| | Needle & Syringe (IM) | 286 | <100 | <100 |
| | | 287 | <100 | <100 |
| | | 288 | | <100 |

TABLE 7

| | | | | | | Pseudovirus | |
|---|---|---|---|---|---|---|---|
| | | | | | | Neutralization | |
| | | | | Serum | | IC50) | |
| | | Serum | | NT Titer | | VN/1203 -- | |
| | Animal | HI Titer | | (MicroNeut) | | IN/05/05 | |
| Group | ID | Week 5 | Week 7 | Week 5 | Week 7 | Week 5 | Week 5 |

| | | | | |
|---|---|---|---|---|
| 21 | 500 μg | 46 | <100 | <100 |
| | Control vector | 47 | <100 | <100 |
| | Agro-jet (ID/SQ) | 48 | <100 | <100 |
| | | 49 | <100 | <100 |
| | | 289 | <100 | <100 |
| | | 290 | <100 | <100 |
| | | 291 | <100 | <100 |
| | | 292 | <100 | <100 |
| 22 | 500 μg | 52 | <100 | 163 |
| | HA (IN/05/05) | 53 | 258 | 600 |
| | HA (Anhui/01/05) | 54 | 209 | 510 |
| | HA (Nigeria/641/05) | 55 | 434 | 1360 |
| | Agro-jet (ID/SQ) | 293 | <100 | <100 |
| | | 294 | <100 | 341 |
| | | 295 | <100 | 122 |
| | | 296 | 114 | 378 |
| 23 | 50 μg | 56 | 258 | 547 |
| | HA (IN/05/05) | 57 | <100 | 225 |
| | HA (Anhui/01/05) | 58 | 504 | 2285 |
| | HA (Nigeria/641/05) | 59 | 119 | 405 |
| | Agro-jet (ID/SQ) | 297 | 139 | 1019 |
| | | 298 | 297 | 696 |
| | | 299 | 337 | 2060 |
| | | 300 | <100 | 122 |
| 24 | 5 μg | L47 | <100 | 152 |
| | HA (IN/05/05) | L67 | <100 | <100 |
| | HA (Anhui/01/05) | L69 | 322 | 1526 |
| | HA (Nigeria/641/05) | L71 | 271 | 1284 |
| | Agro-jet (ID/SQ) | 78 | <100 | 819 |
| | | 79 | <100 | <100 |
| | | 80 | <100 | 819 |
| | | 81 | <100 | <100 |
| 25 | 0.5 μg | L72 | <100 | <100 |
| | HA (IN/05/05) | L73 | <100 | 130 |
| | HA (Anhui/01/05) | L74 | <100 | <100 |
| | HA (Nigeria/641/05) | L75 | <100 | <100 |
| | Agro-jet (ID/SQ) | L99 | 157 | 908 |
| | | 26 | <100 | <100 |
| | | 82 | <100 | <100 |
| | | 96 | <100 | <100 |

FIG. 10A depicts neutralization against Indonesia (IN/05/05) pseudovirus. Neutralization by sera pool from chicken groups immunized with no insert vector was included as Control vector (as in Table 3A, Group 1) (first upper row); A/Indonesia/05/2005 as IN/05/05 (as in Table 3A, Group 2) (second upper row); a combination of three DNA constructs expressing A/Indonesia/05/2005 as IN/05/05, A/Anhui/1/2005 as Anhui/01/05, and A/Vietnam/1203/04 as VN/1203/04 HA (as in Table 3A, Group 3) (third row); and A/Indonesia/05/2005 as IN/05/05 (as in Table 3A, Group 6) (last row) was assessed by incubation of chicken sera with A/Indonesia/05/2005 HA NA pseudotyped lentiviral vectors encoding luciferase. Only four groups are shown, but the pattern of immune response was similar and comparable in all six groups (as in Table 3A, Group 1 to 6). There was a progressive increase in the potency of immunization after each DNA vaccination with maximal response at week 7 (shown). 100% neutralization was seen in dilutions up to 1:1600 and >90% neutralization in dilutions up to 1:3200. Bird # 238 consistently showed a ‘low’ level of neutralization in all the groups, but was fully protected against viral challenge. Percent neutralization was calculated by the reduction of luciferase activity relative to the values achieved in the absence of sera.

FIG. 10B depicts neutralization against Vietnam (VN/1203/04) pseudovirus. The sera were incubated with A/Vietnam/1203/2004 HA NA pseudotyped lentiviral vectors encoding luciferase. The potency of the response was high, but not as high as column A (against IN/05/05 pseudovirus). The responses were comparable in dilutions up to 1:800.

FIG. 10C depicts neutralization against Anhui (Anhui/01/05) pseudovirus. The sera were incubated with A/Anhui/1/2005 HA NA pseudotyped lentiviral vectors encoding luciferase. The potency of the response was high, similar to Indonesia (column A), particularly in groups immunized with univalent HA and immunized with trivalent HA.

FIG. 10D depicts neutralization against Nigeria (Nigeria/641/05) pseudovirus. The sera from the indicated 4 groups were incubated with A/chicken/Nigeria/641/2006 HA NA pseudotyped lentiviral vectors encoding luciferase. We noted that the potency of the response was high, but not as high as column A.

Immunization of birds with the control DNA construct (CMV/R) without HA-specific insert generated minimal nonspecific immune responses using the pseudotype neutralization assays and HI titers up to 1:40 measured 1 week after 3 DNA immunizations. In contrast, birds immunized with univalent and multivalent vaccines generated a significant and robust immune response neutralizing up to 100% using the pseudotype lentiviral assays and had high levels of serum HI antibodies (1:1280 HI titers) (FIG. 10A-D and Table 5). In general, there was a progressive increase in the amount of neutralization after each successive DNA vaccination with maximal response at 1 week after the 3rd DNA immunization (FIG. 10A-D), especially in the multivalent vaccine group delivered ID/SC using the Agro-Jet®, univalent vaccine using the IM route and using the Agro-Jet®. Neutralization of Indonesia pseudotype virus was the most robust, with near 100% neutralization in dilutions as high as 1:3200. While homologous virus neutralization was generally comparable to heterologous neutralization (FIGS. 10B, 10C, and 10D), only the multivalent vaccine group presented homologous neutralization; the rest of the groups are heterologous for Vietnam, Anhui and Nigeria. Interestingly, neutralization against heterologous virus Nigeria was better than some homologous neutralization (FIG. 10B). Similar responses were noted in the other groups, including administration of univalent HA-Indonesia subcutaneously by needle syringe and via Agro-Jet® (FIG. 7). In the Multivalent vaccine group only bird # 208 did not produce a high neutralizing antibody titer, although it produced almost the same degree of neutralization at each time point and was protected.

### Example 8

### Protection of Chickens Against Heterologous H5N1 Vietnam 1203/04 Virus Challenge

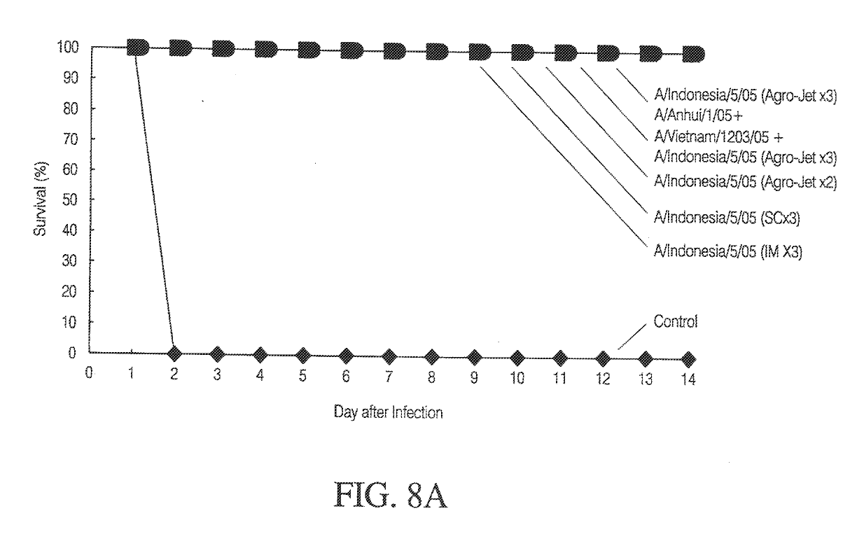

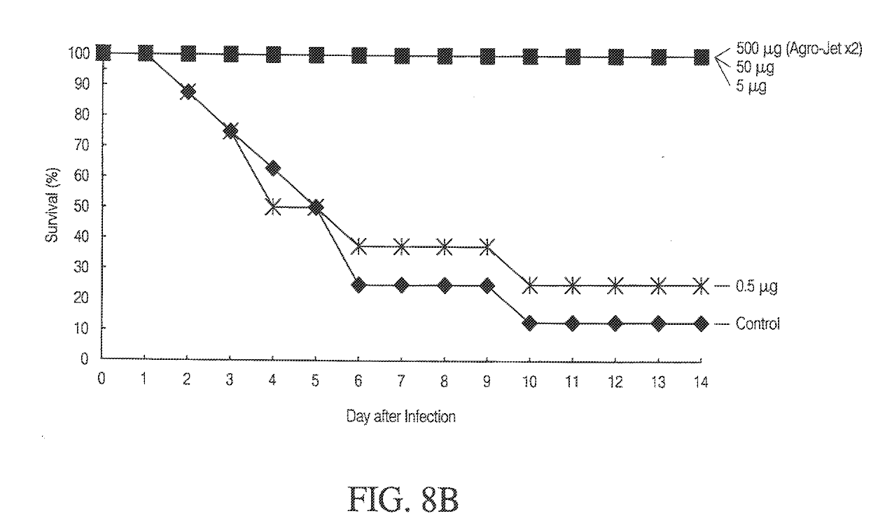

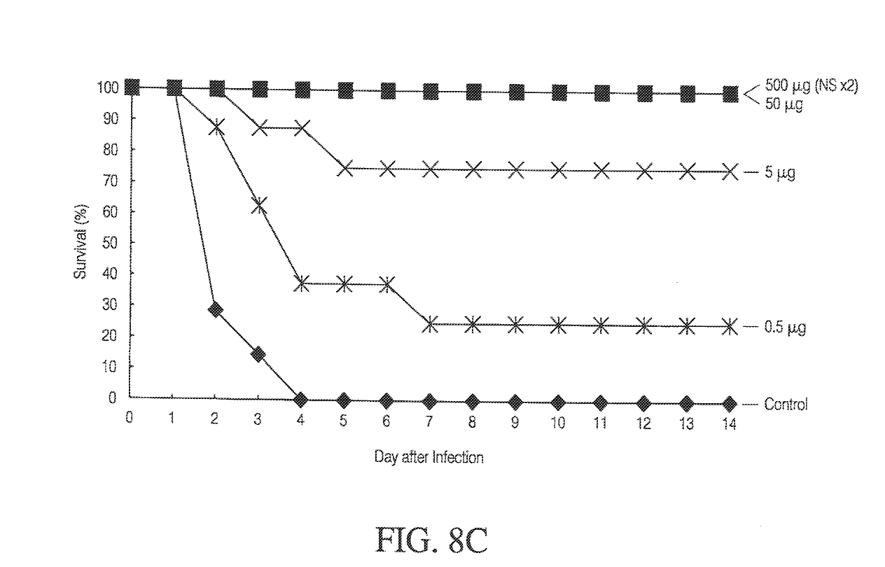

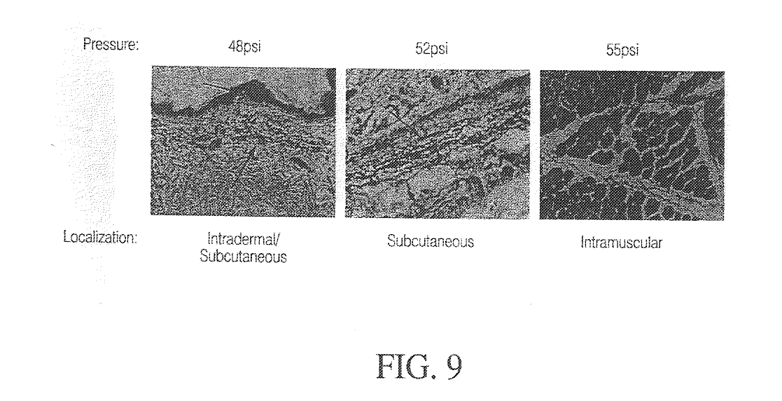

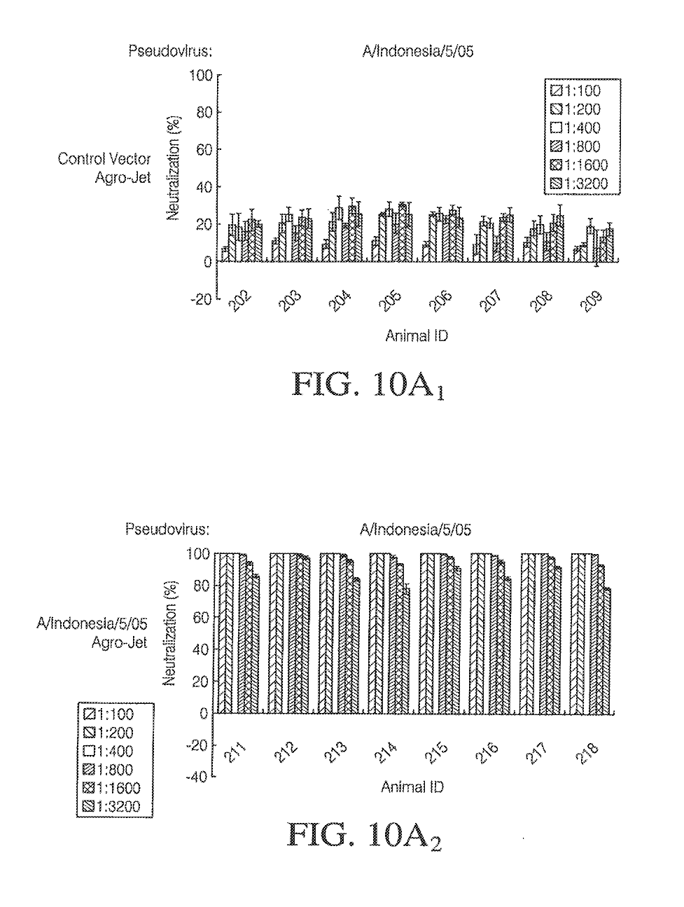

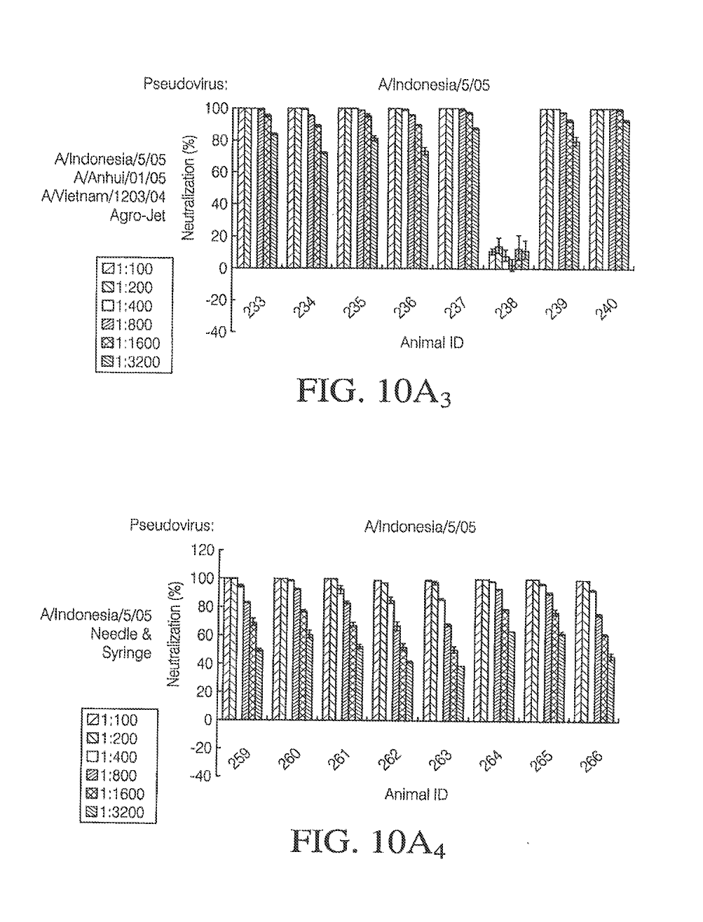

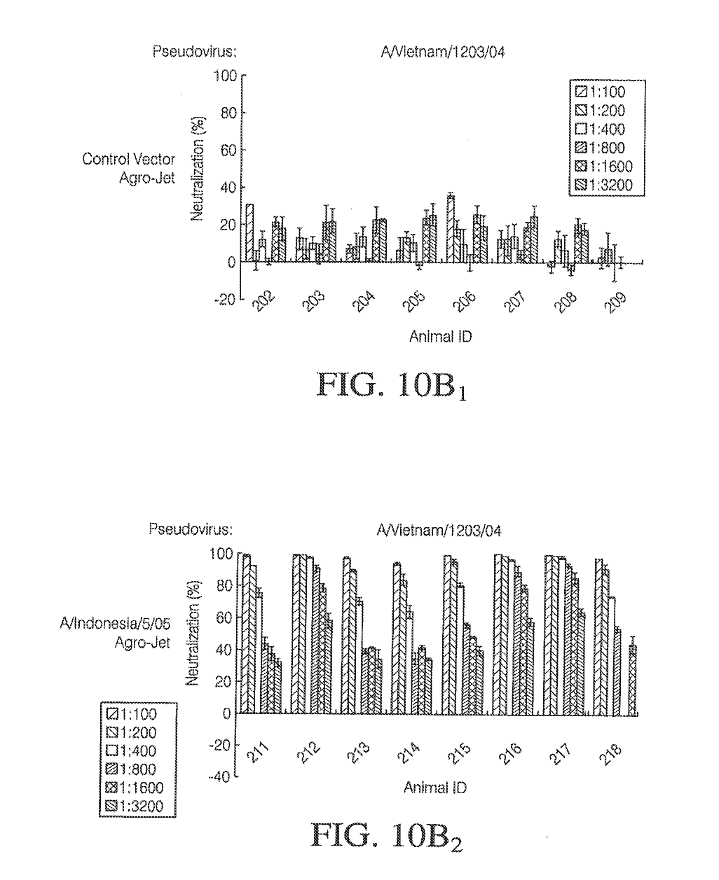

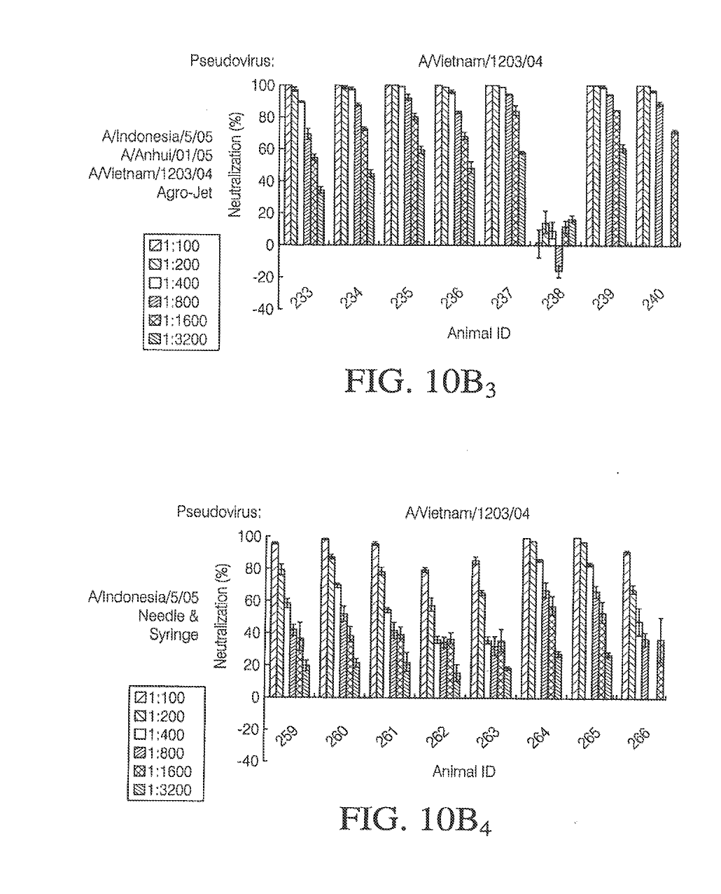

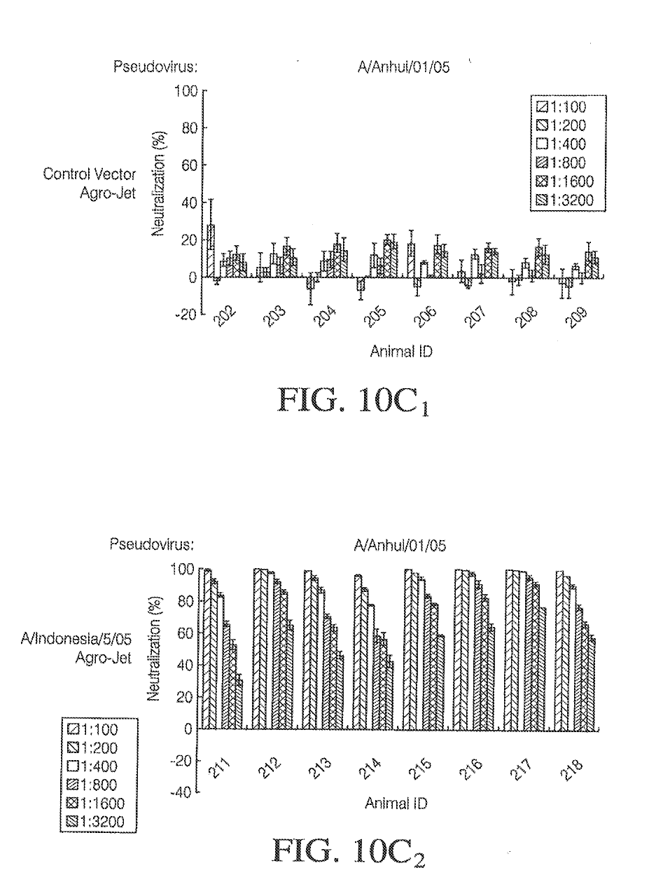

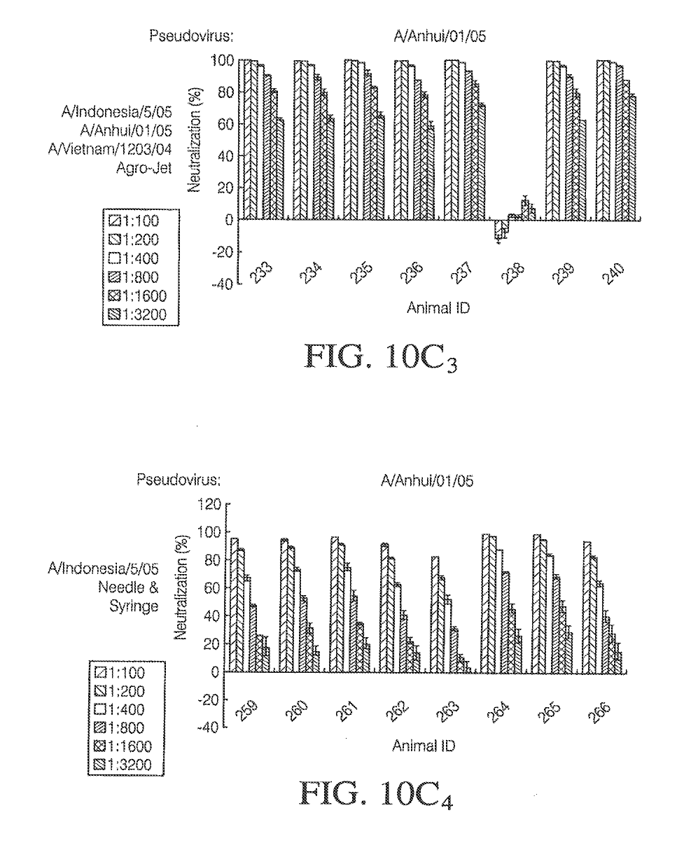

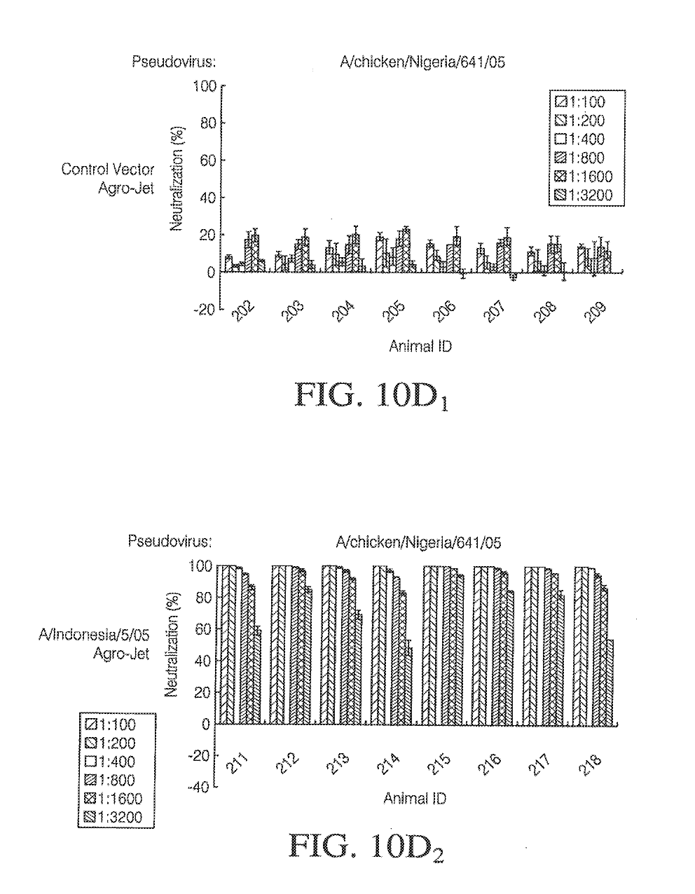

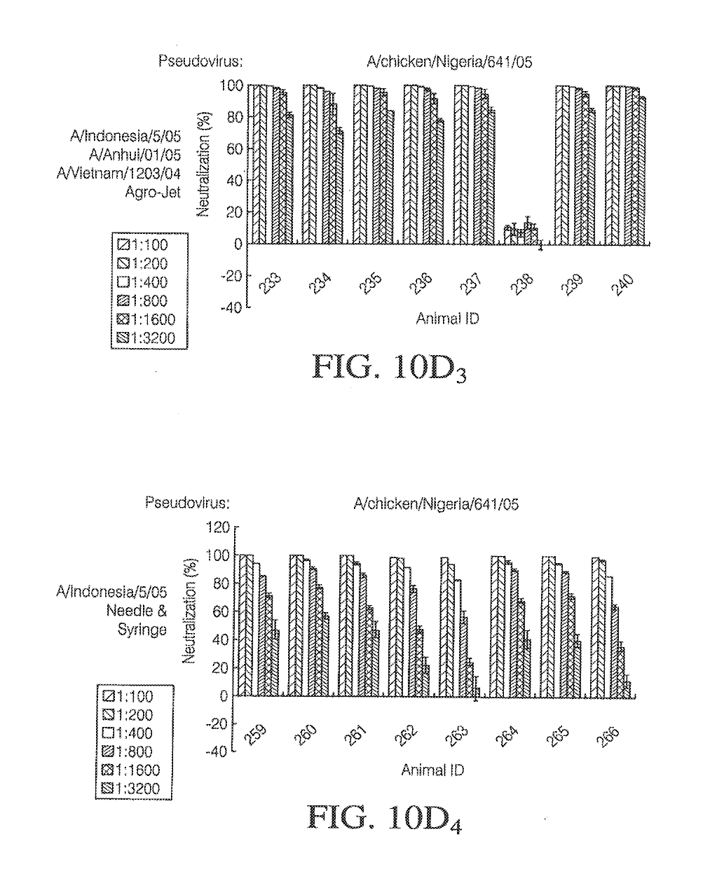

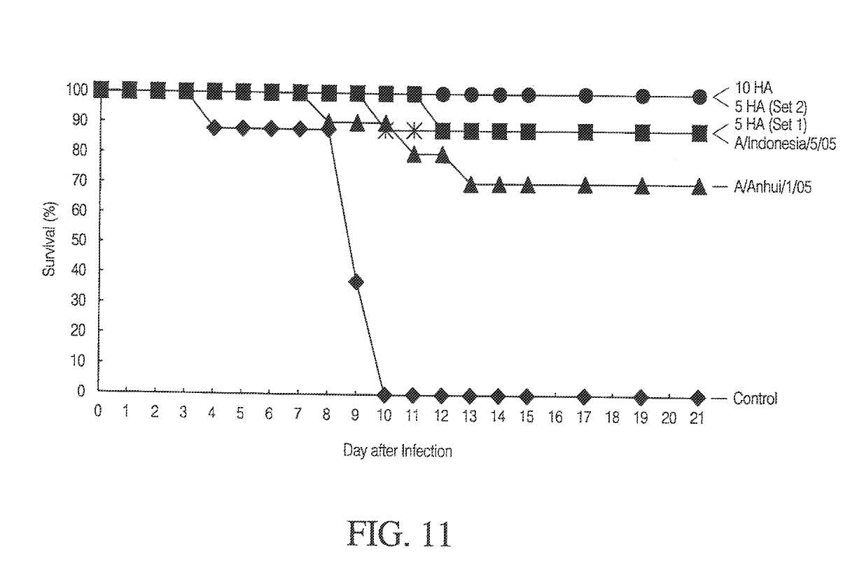

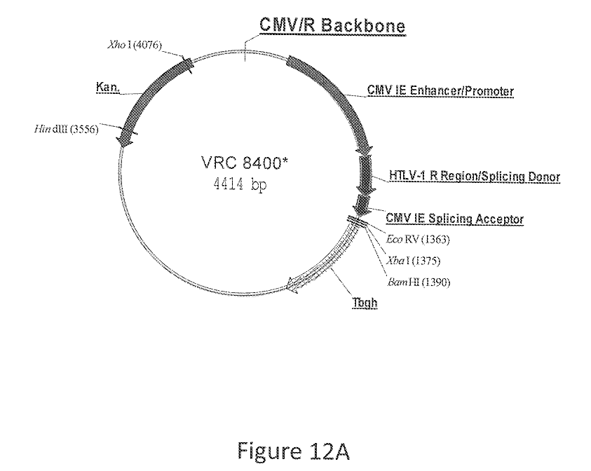

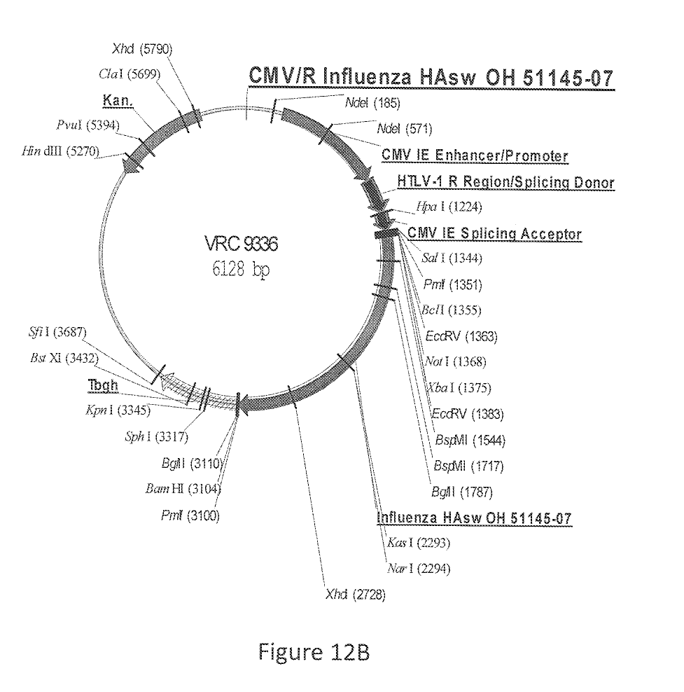

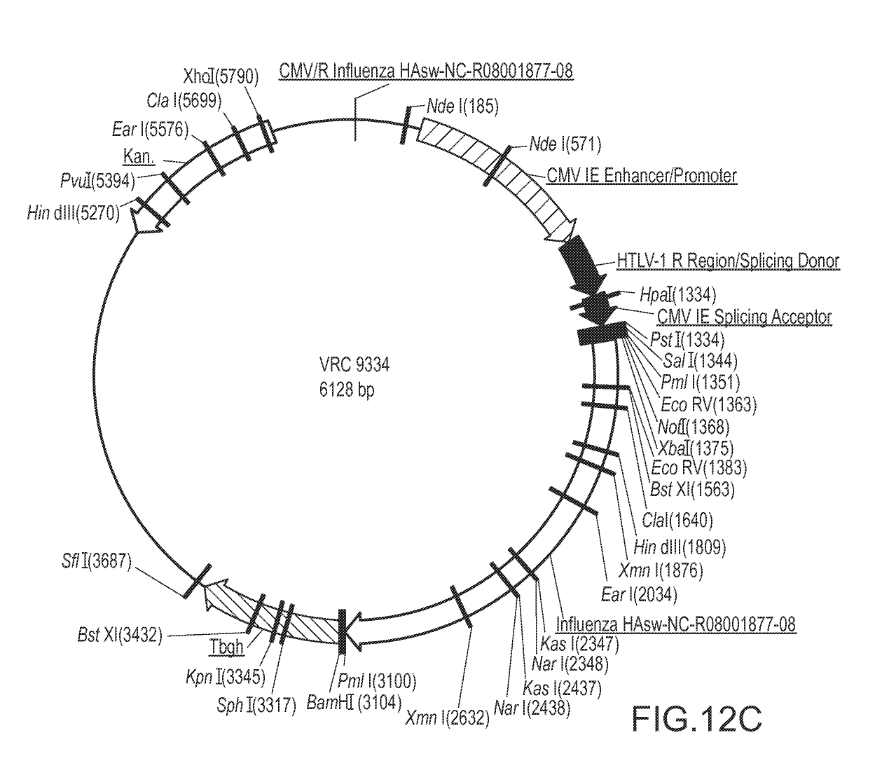

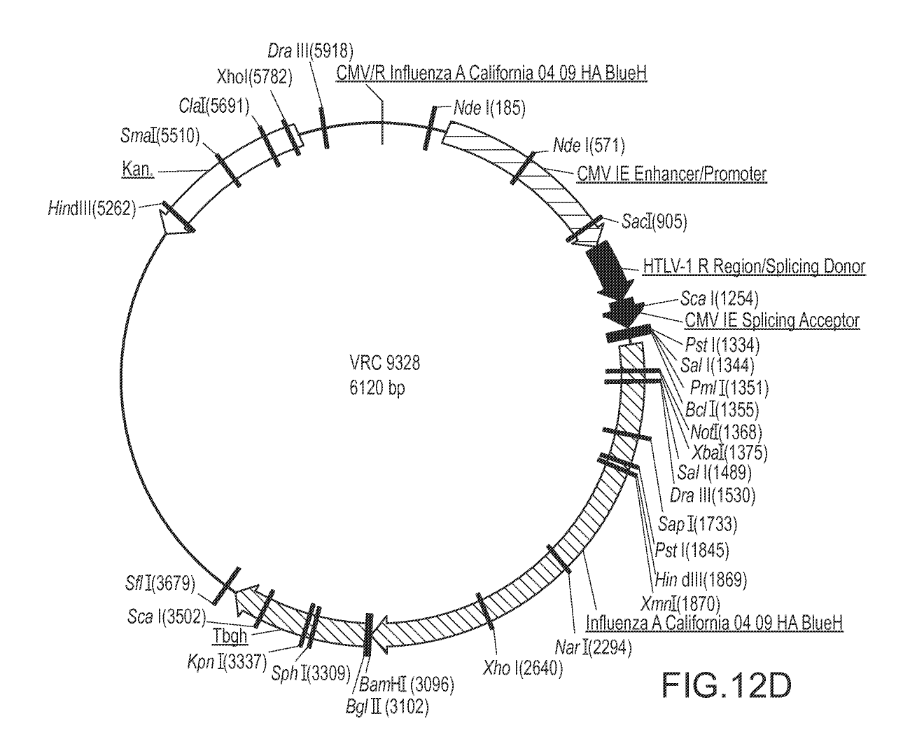

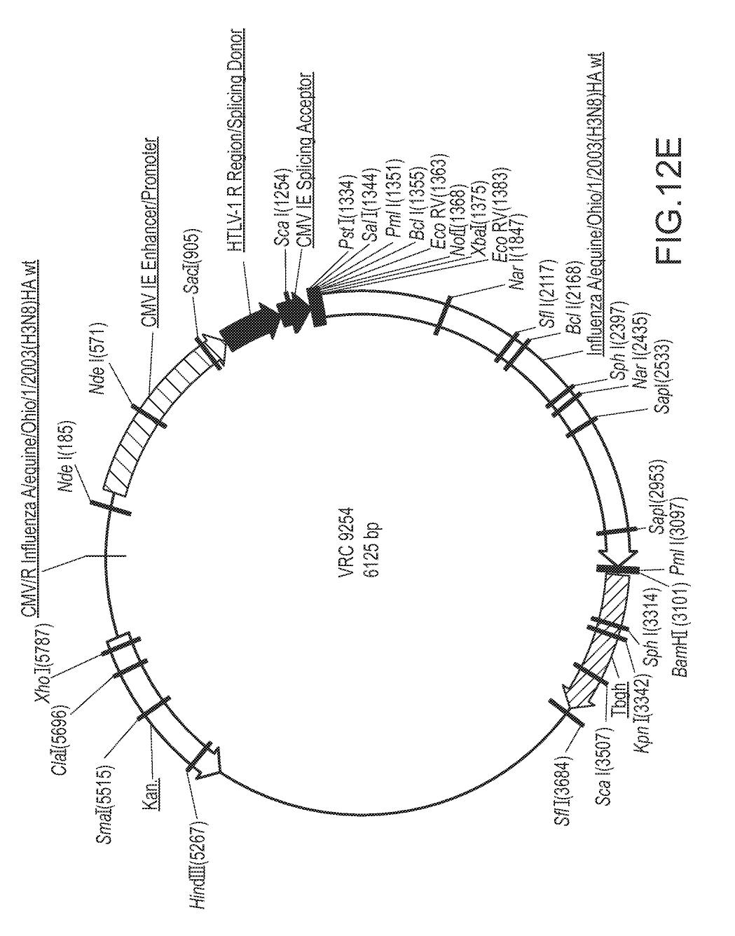

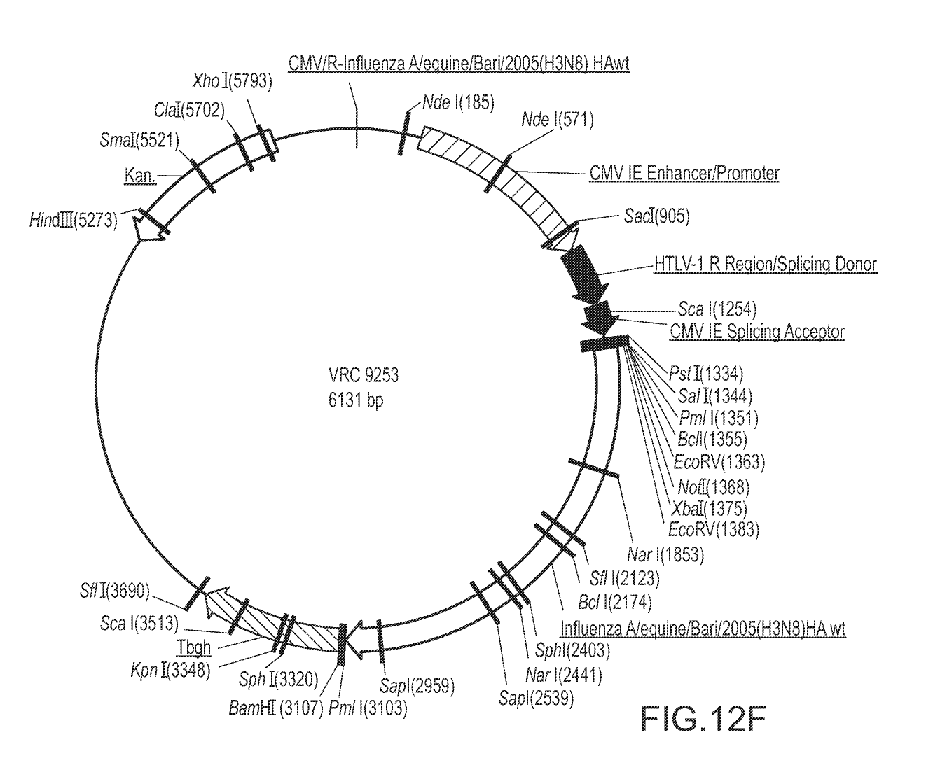

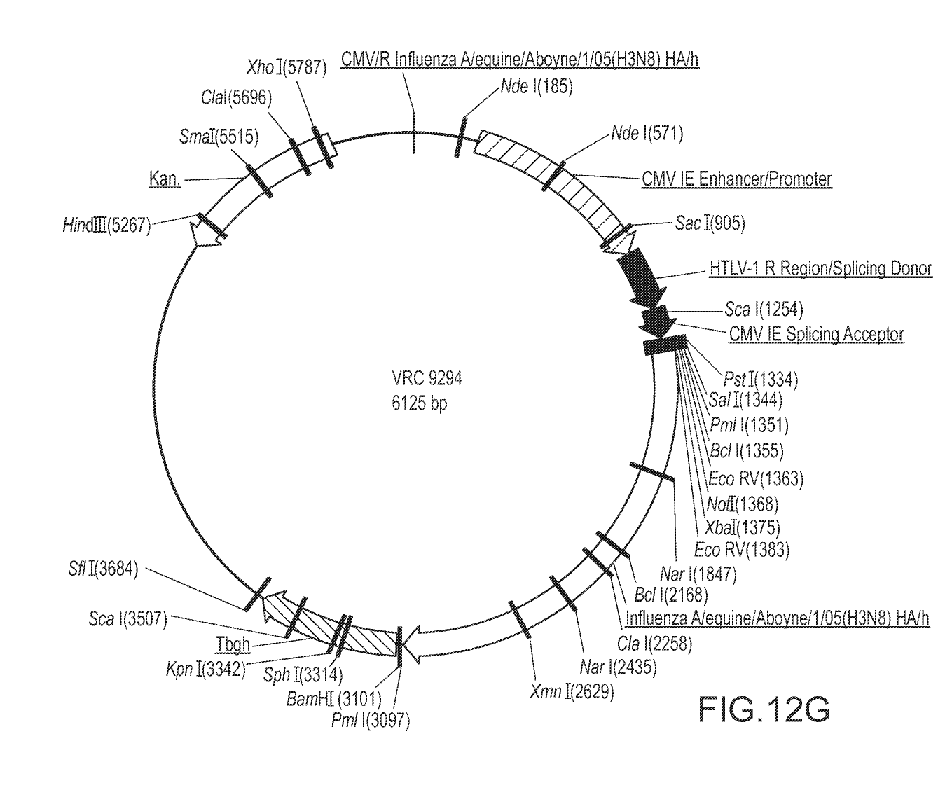

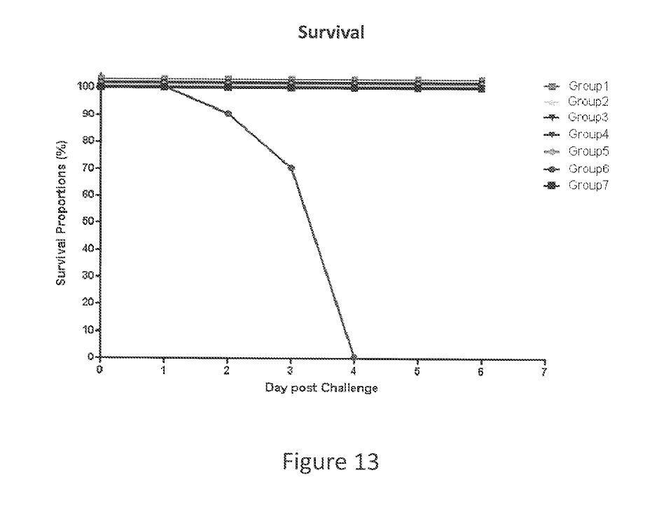

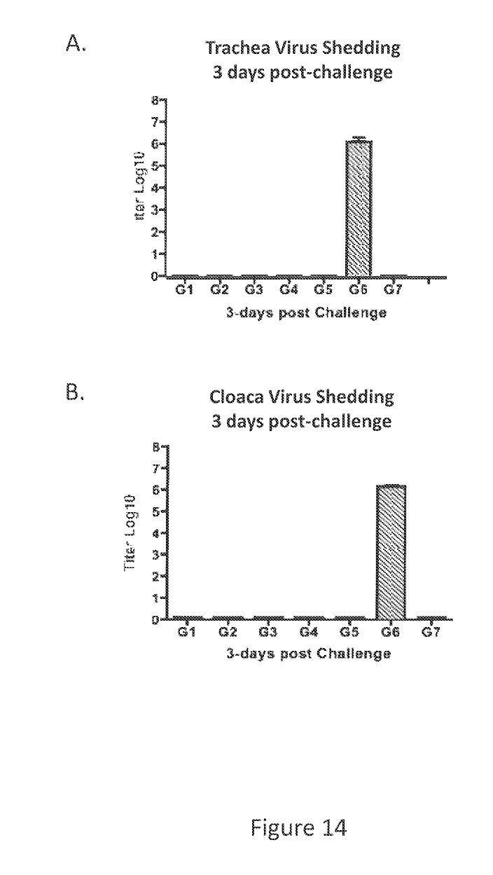

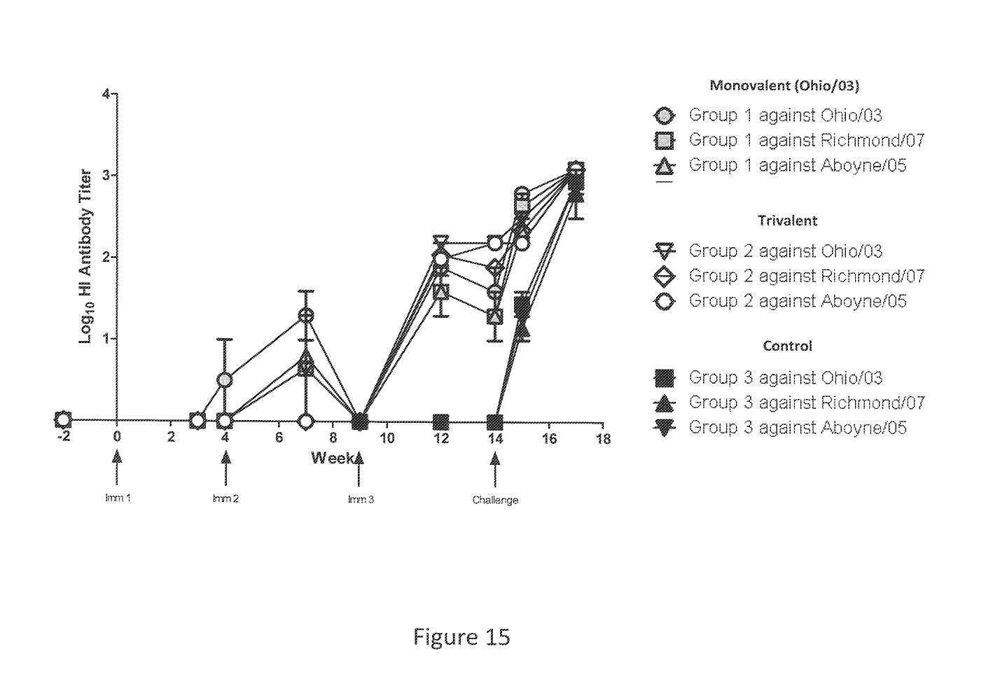

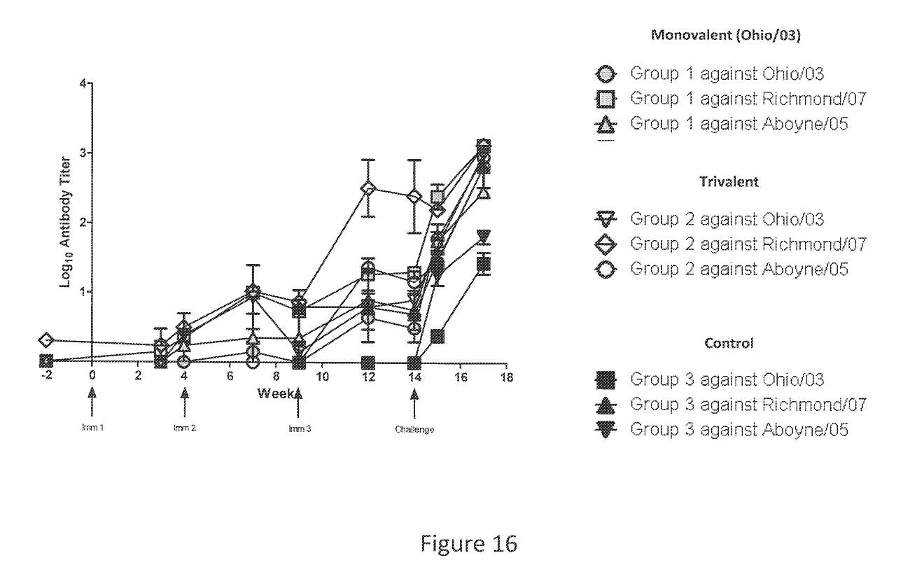

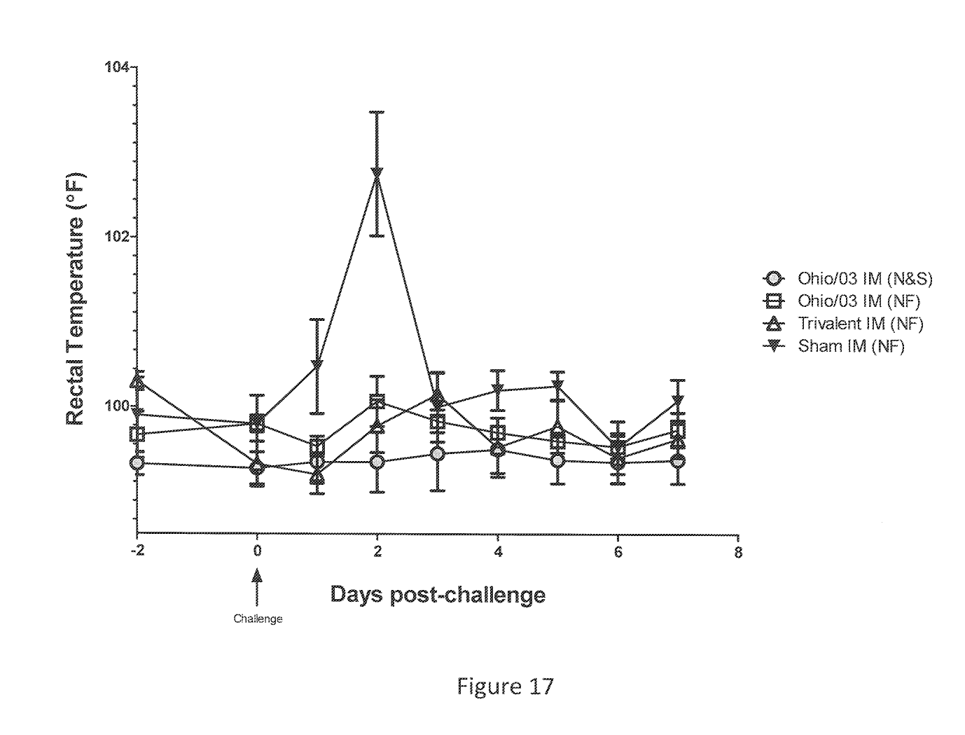

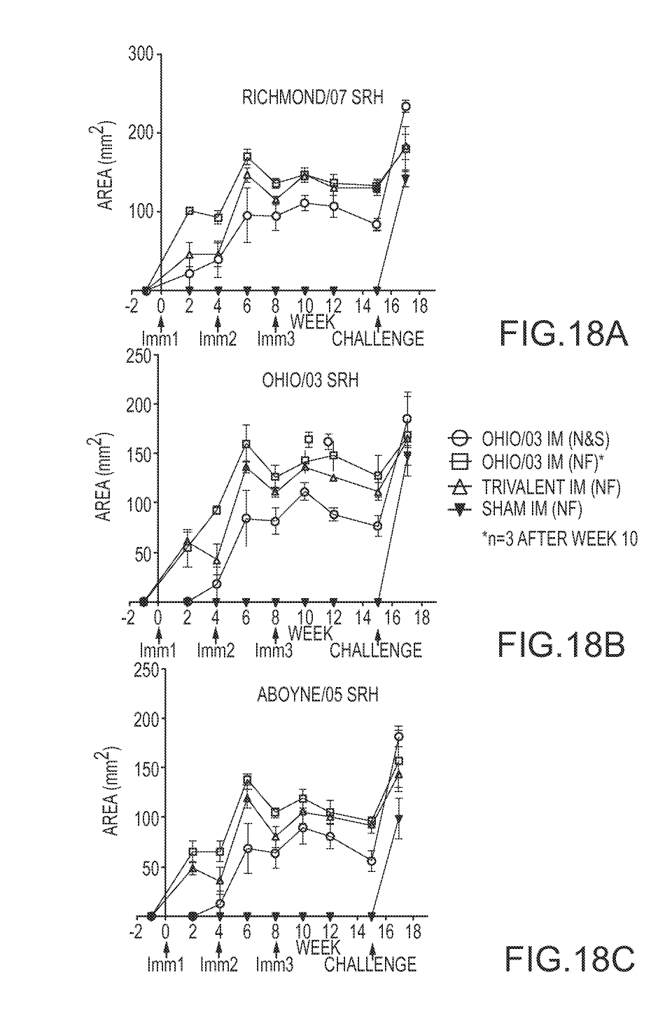

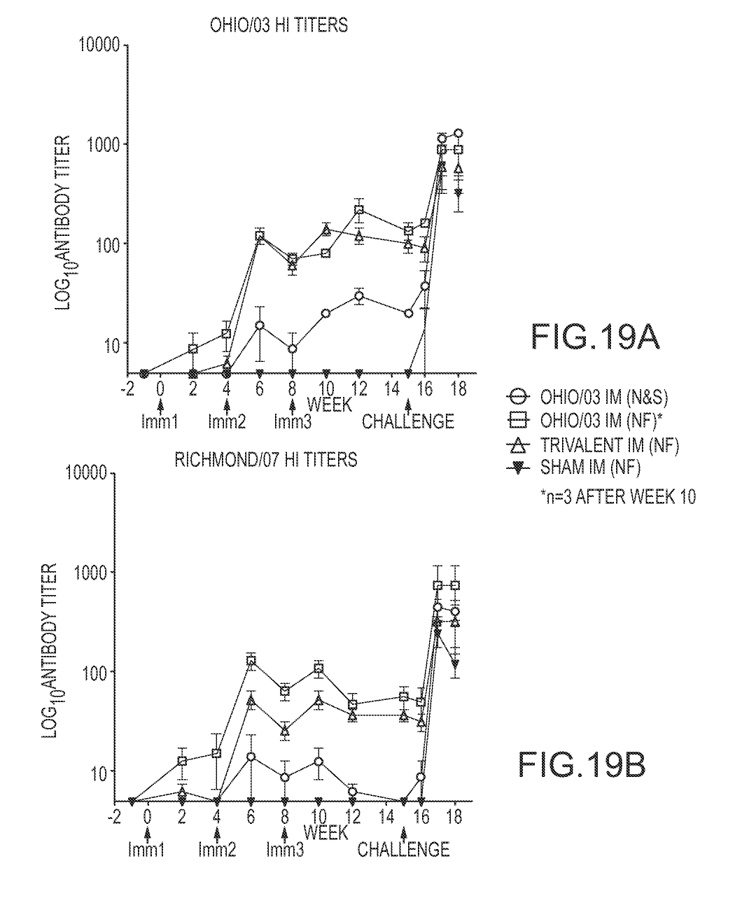

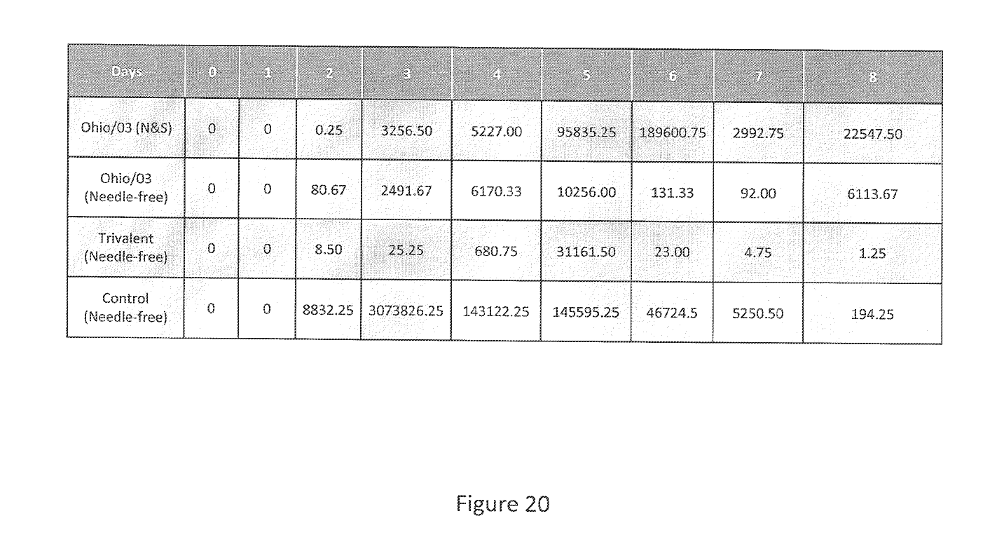

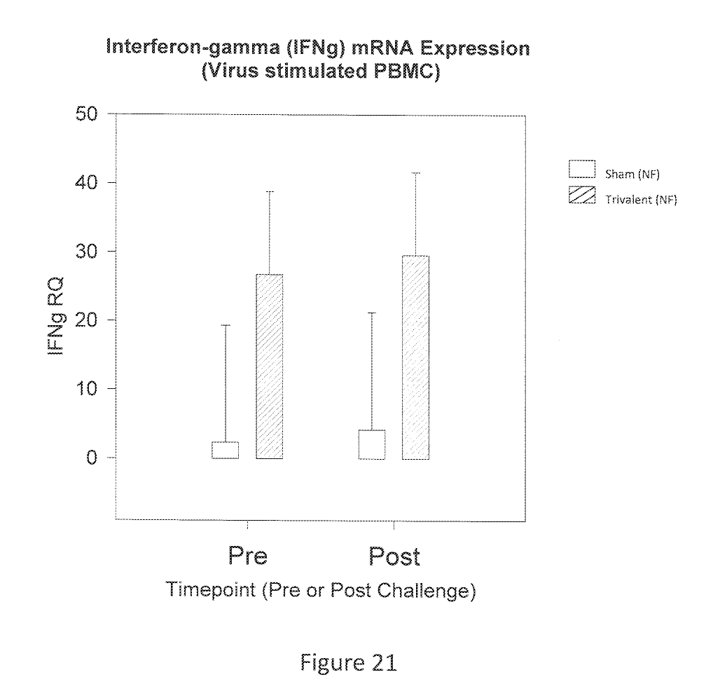

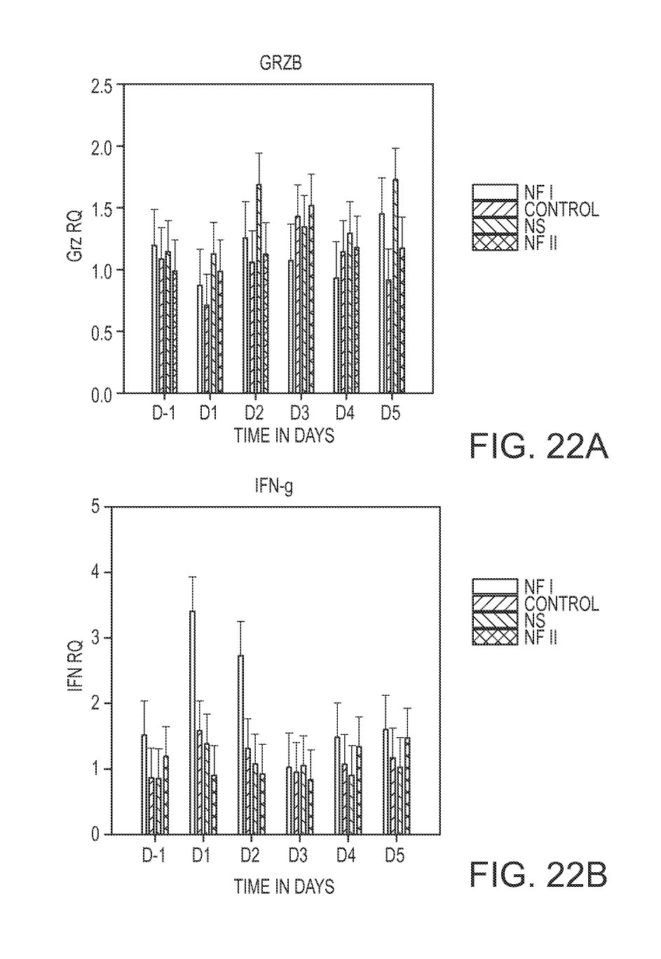

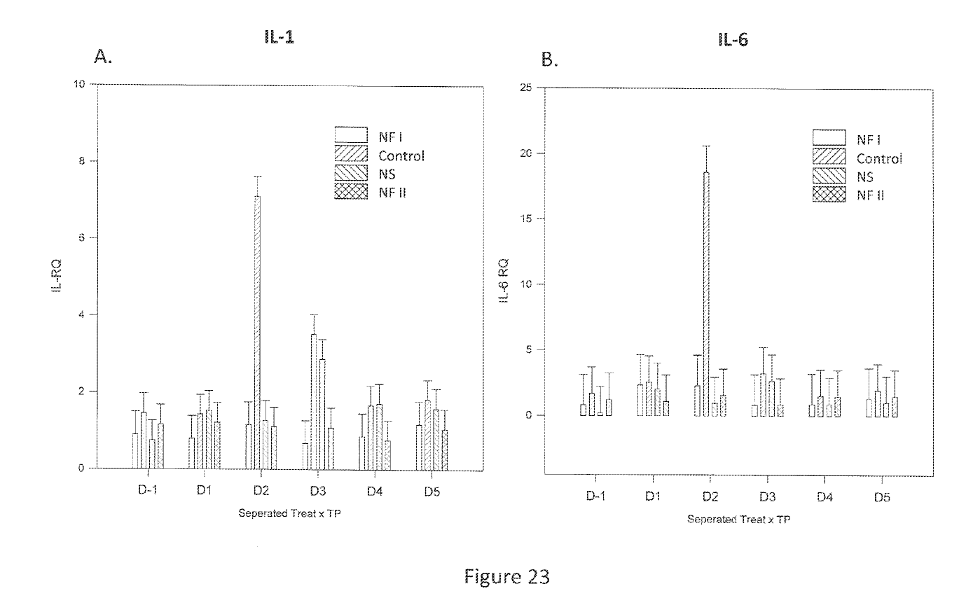

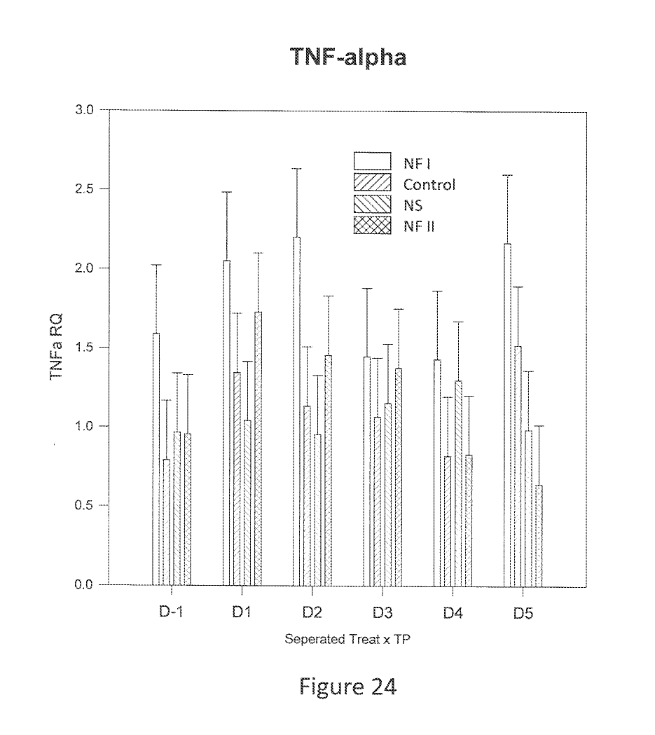

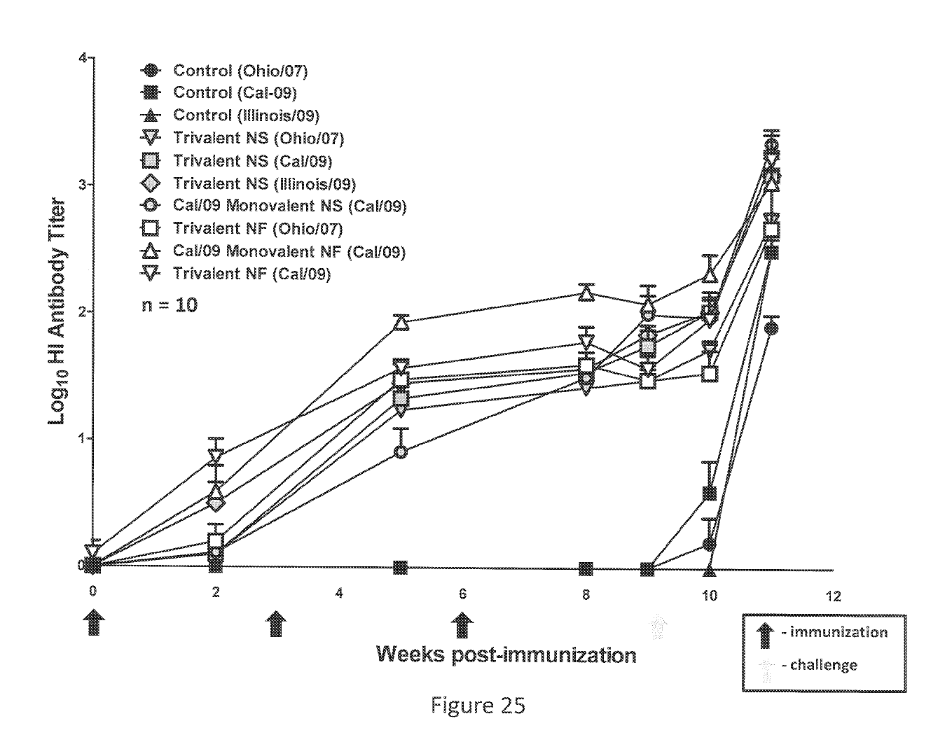

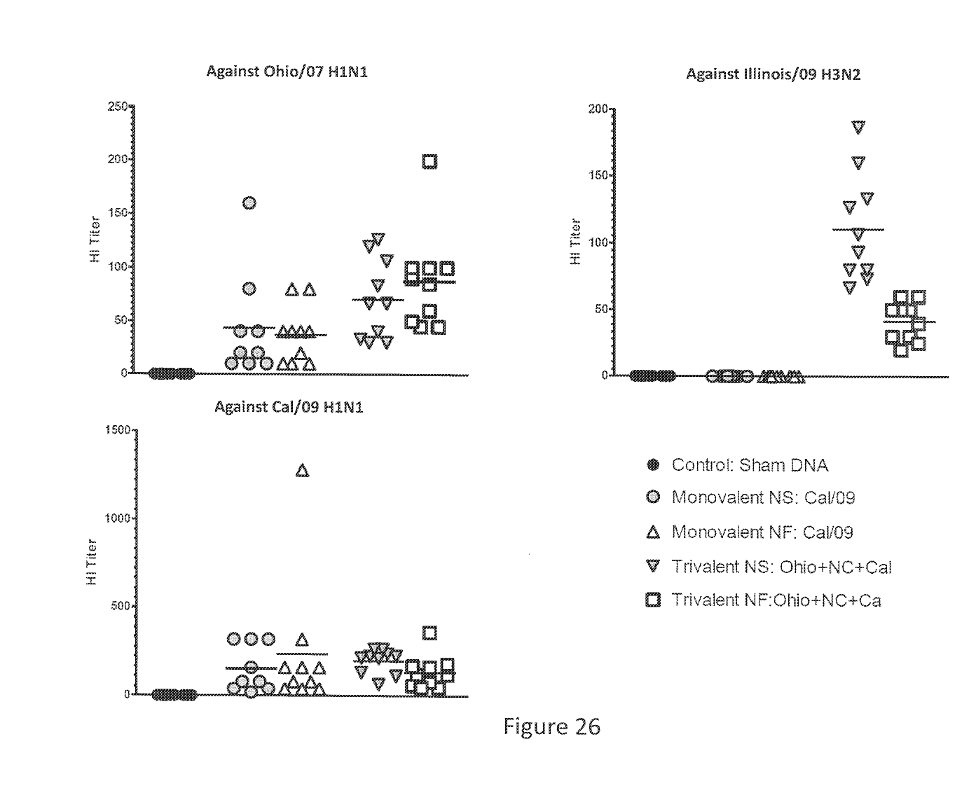

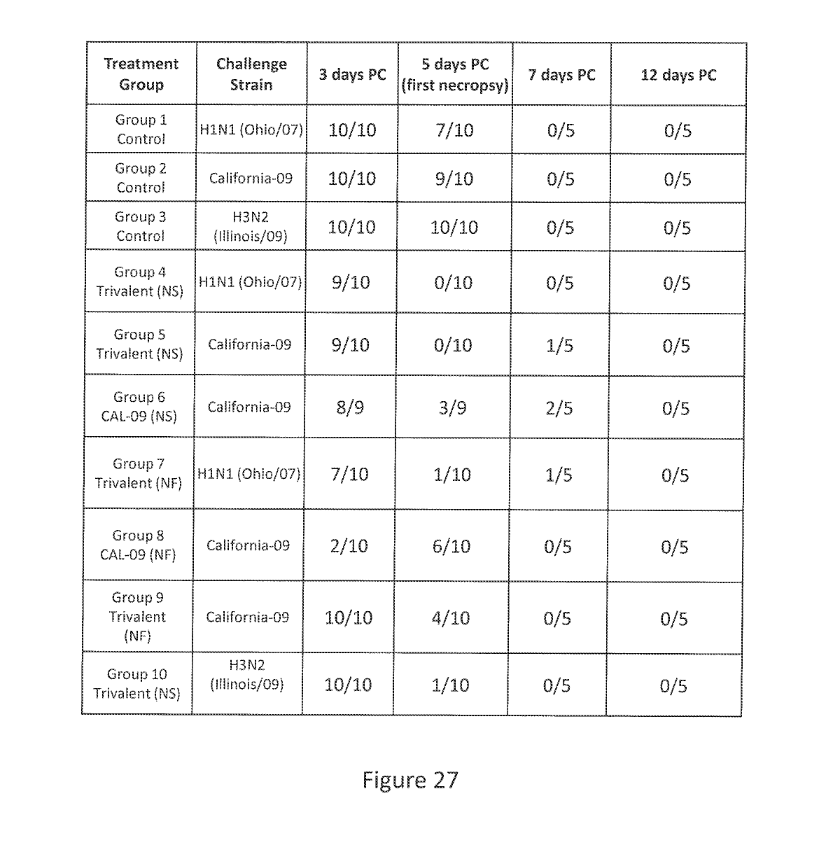

To determine the extent of cross protection by our univalent and multivalent vaccine candidates, immunized chickens were challenged with 100 LD₅₀ of HPAI-H5N1 Vietnam 1203/04 heterologous virus intranasally via choanal slit using standard methods as described in Webster et al., Characterization of H5N1 influenza viruses that continue to circulate in geese in southeastern China. J Virol (2000) 76: 118-126 and Hulse et al., Molecular determinants within the surface proteins involved in the pathogenicity of H5N1 influenza viruses in chickens. J Virol (2000) 78: 9954-9964, herein incorporated by reference in their entirety, and monitored for morbidity, mortality, viral shedding and serum antibodies. A/Vietnam/1203/04 (H5N1) (A/VN/1203/04) was obtained from the repository at the Centers for Disease Control and Prevention (CDC), Atlanta, Ga. The virus was propagated in 10-day old embryonated chicken eggs at 35° C. and stored at −70° C. until use. The virus was titrated by the Reed and Muench method to determine EID₅₀ Reed L J, et al. (1938) Am J Hygiene 27: 493-497. While all the control animals (empty vector vaccine) died within 2 days of infection, 100% survival was noted in the rest of the birds (FIG. 8A). The animals that were healthy (showed no signs of clinical disease or malaise) were euthanized on day 14.

Birds immunized as described in Example 6 were challenged one week after the last immunization via intranasal inoculation of A/Vietnam/1203/2004 strain of H5N1 influenza virus in the BSL-3+ facility (Avrum Gudelsky Center, University of Maryland, College Park) using methods described in Webster et al., Characterization of H5N1 influenza viruses that continue to circulate in geese in southeastern China, J Virol (2002) 76: 118-126; Hulse et al., Molecular determinants within the surface proteins involved in the pathogenicity of H5N1 influenza viruses in chickens, J Virol (2004) 78: 9954-9964; and Makarova et al., Replication and transmission of influenza viruses in Japanese quail, Virology (2003) 310: 8-15, herein incorporated by reference in their entirety.

Vaccinated chickens were inoculated with 20 LD₅₀ of highly pathogenic A/Vietnam/1203/2004 heterologous virus intranasally using standard methods and monitored for morbidity, mortality, viral shedding and serum antibodies.

Specifically, white Leghorn chickens were challenged one week after the last immunization with 20 lethal dose 50 (LD₅₀) of A/Vietnam/1203/04 (H5N1) influenza A virus, equivalent to 2×10⁴ EID₅₀ based on previous challenges. Song H, et al. (2007) J Virol 81: 9238-9248. Chickens were infected with 200 μl virus intranasally. Tracheal and cloacal swabs were collected days 3 and 5 post-challenge and stored in glass vials containing BHI medium (BBL™ Brain Heart Infusion, Becton Dickinson) at −80° C. Blood was collected 14 days post-challenge and serum was titered by microneutralization assay. Chickens were observed and scored daily for clinical signs of infection, morbidity and mortality. Chickens that survived the study were bled and humanely euthanized at day 14 post-challenge. Lungs, heart, intestine and kidney were collected and samples were stored in formalin for histopathology. Experiments were carried out under BSL3+ conditions with investigators wearing appropriate protective equipment and compliant with all Institutional Animal Care and Use Committee-approved protocols and under Animal Welfare Act regulations at the University of Maryland, College Park, Md.

While all the control animals died within 2 days of infection, 100% survival was noted in the rest of the birds (FIG. 8). The animals that were healthy, showing no signs of clinical disease or malaise, were euthanized on day 14. Representative tracheal and cloacal swabs were chosen to run an EID₅₀ assay for comparison and virus titers were determine by the method of Reed and Meunch. Briefly, swabs were used to infect 10 day-old embryonated chicken eggs in 10-fold dilutions. Three eggs were inoculated per dilution and incubated for 48 hours before titration. There was no evidence for viral shedding monitored via tracheal and cloacal swabs of infected birds 2-14 days after infection.

### Example 9

### Relative Potency of DNA Vaccines Delivered by Needle or Needle-Free Injection Devices

To compare the relative efficacy of DNA vaccines delivered intramuscularly by needle and syringe versus the needle-free Agro-Jet® device injection, a dose-response study was performed with decreasing amounts of DNA vaccine ranging from 500 to 0.5 μg with two inoculations.

The HA derived from A/chicken/Nigeria/641/2006 was substituted for A/Vietnam/1203/2004 since it represented a more contemporary isolate. Five groups were immunized with the trivalent HA vaccine (A/Anhui/1/2005, A/Indonesia/05/2005, and A/chicken/Nigeria/641/2006) using 500 μg (167 μg of each of the three DNA constructs), 50 μg (17 μg of each DNA construct), 5 μg (1.7 μg of each DNA construct), 0.5 μg (0.17 μg of each DNA construct) and a 500 μg control vector administered intramuscularly with needle and syringe. Another five groups were given the same DNA vaccine doses by the ID/SC route using Agro-Jet®.

The observed rate of protection was higher among the animals receiving 5 μg by Agro-Jet (8/8) than by IM injection (6/8) (FIG. 8, B vs. C). Both modes provided complete protection for all animals at doses higher than this, and 25% protection for the animals receiving 0.5 μg doses (FIGS. 8B and 8C). Survival differences between consecutive doses were assessed using a log-rank test on the survival curves for pairs of groups. A test was deemed significant if the p-value was <0.01, and marginally significant if the p-value was <0.05 but >0.01. Chickens injected IM showed a marginally significant difference between 0.5 and 5 μg (p=0.047). In the same group there was a significant difference between control and 5, 50 and 500 μg (p<0.001 for all comparisons) and the difference between control and 0.5 μg was marginally significant (p=0.016). Chickens that were injected using Agro-Jet® showed a significant difference between 0.5 and 5 μg (p=0.004) and between control and 5, 50, and 500 μg (p<0.001 for all comparisons). There were no differences between control and 0.5 μg or between 5, 50, and 500 μg. Lastly, the survival differences between Agro-Jet® and IM for each dose group were not significant. The neutralizing antibody response to homologous and heterologous HAs corresponded with protection and correlated with dose, with higher titers elicited by injection with Agro-Jet® compared to needle (Table 8). We assessed viable viral shedding after inoculation by chick embryo inoculation three days after virus challenge (Week 8). While we noted some embryonic lethality at the 0.5 μg dose, there was no embryonic lethality at 5, 50 or 500 μg groups.

TABLE 8

| | LAI Titer (Week 5) | | | | | |
|---|---|---|---|---|---|---|
| | Agro-Jet ® | | | Needle & Syringe | | |
| Immunogen | Animal ID | VN/1203 | IN/05/05 | Animal ID | VN/1203 | IN/05/05 |
| 500 μg | 46 | <100 | <100 | 27 | 102 | 109 |
| Control | 47 | <100 | <100 | 28 | <100 | <100 |
| | 48 | <100 | <100 | 29 | <100 | <100 |
| | 49 | <100 | <100 | 268 | <100 | <100 |
| | 289 | <100 | <100 | 269 | 150 | 134 |
| | 290 | <100 | <100 | 270 | <100 | <100 |
| | 291 | <100 | <100 | 271 | <100 | <100 |
| | 292 | <100 | <100 | | | |
| 500 μg | 52 | <100 | 163 | 30 | 125 | 167 |
| A/Indonesia/05/05 | 53 | 258 | 600 | 31 | <100 | 115 |
| A/Anhui/01/05 | 54 | 209 | 510 | 32 | <100 | 142 |
| A/c/Nigeria/641/05 | 55 | 434 | 1360 | 33 | <100 | <100 |
| | 293 | <100 | <100 | 272 | 169 | 181 |
| | 294 | <100 | 341 | 273 | <100 | 142 |
| | 295 | <100 | 122 | 274 | <100 | <100 |
| | 296 | 114 | 378 | 275 | 194 | 614 |
| 50 μg | 56 | 258 | 547 | 34 | <100 | 131 |
| A/Indonesia/05/05 | 57 | <100 | 225 | 35 | <100 | 145 |
| A/Anhui/01/05 | 58 | 504 | 2285 | 36 | <100 | <100 |
| A/c/Nigeria/641/05 | 59 | 119 | 405 | 37 | <100 | <100 |
| | 297 | 139 | 1019 | 277 | <100 | <100 |
| | 298 | 297 | 696 | 278 | <100 | 140 |
| | 299 | 337 | 2060 | 279 | <100 | <100 |
| | 300 | <100 | 122 | 280 | <100 | <100 |
| 5 μg | L47 | <100 | 152 | 38 | 125 | 149 |
| A/Indonesia/05/05 | L67 | <100 | <100 | 39 | <100 | <100 |
| A/Anhui/01/05 | L69 | 322 | 1526 | 40 | <100 | <100 |
| A/c/Nigeria/641/05 | L71 | 271 | 1284 | 41 | <100 | <100 |
| | 78 | <100 | 819 | 281 | 144 | 111 |
| | 79 | <100 | <100 | 282 | <100 | <100 |
| | 80 | <100 | 819 | 283 | <100 | <100 |
| | 81 | <100 | <100 | 284 | <100 | <100 |
| 0.5 μg | L72 | <100 | <100 | 43 | <100 | <100 |
| A/Indonesia/05/05 | L73 | <100 | 130 | 44 | <100 | <100 |
| A/Anhui/01/05 | L74 | <100 | <100 | 50 | <100 | <100 |
| A/c/Nigeria/641/05 | L75 | <100 | <100 | 285 | <100 | <100 |
| | L99 | 157 | 908 | 286 | <100 | <100 |
| | 26 | <100 | <100 | 287 | <100 | <100 |
| | 82 | <100 | <100 | 288 | <100 | <100 |
| | 96 | <100 | <100 | | | |

### Example 10

This Example demonstrates the effect of adjuvant on the immune response resulting from DNA vaccination of chickens using DNA constructs of the present invention.

This study was conducted using the methodology described in Example 7. Briefly, seven groups of ten chickens each were vaccinated using a DNA vaccine with or without adjuvant. The DNA vaccine was a multivalent vaccine consisting of the DNA constructs CMV/R IN-HA, CMV/R NIG-HA and CMV/R AN-HA. The control vaccine consisted of the CMV/R vector without any insert. In vaccine doses comprising adjuvant, the DNA constructs were mixed with VAXFECTIN™ (Vical, Inc., Sand Diego, Calif.) according to the manufacturer's instructions. All vaccines were administered using a needle-free device (e.g., AGRO-JET® needle-free injector (Medical International Technologies, Montreal, Quebec, Canada)). A summary of the study design is shown in Table 9.

TABLE 9
Adjuvant Study Design

| | No. of | Treatment/Dose | |
|---|---|---|---|
| Group | Chickens | (100 ul bilateral-200 ul total) | |
| 1 | 10 | Trivalent vaccine + VAXFECTIN ™ | 50 ug |
| 2 | 10 | Trivalent DNA | 50 ug |
| 3 | 10 | Trivalent vaccine + VAXFECTIN ™ | 5 ug |
| 4 | 10 | Trivalent DNA | 5 ug |
| 5 | 10 | Trivalent vaccine + VAXFECTIN ™ | 0.5 ug |
| 6 | 10 | Trivalent vaccine | 0.5 ug |
| 7 | 10 | Monovalent control DNA | 50 ug |

Trivalent vaccine comprised the DNA constructs CMV/R Influenza A/Indonesia/05/05 (H5N1) HA-mutA+CMV/R Influenza A/Nigeria/641/2006/(H5N1) HA-mutA+CMV/R Influenza A/Anhui/1/2005/(H5N1) HA-mutA

### Control DNA=CMV/R

Vaccines were administered on Day 0 and 5 weeks
At eight weeks post-vaccination, all groups were challenged intranasally with 5×10⁶ EID₅₀/ml of A/Vietnam/1203/2004 H5N1 virus. On day 3 post challenge, cloacal and tracheal swabs were collected and titrated for virus infectivity in embryonated chicken eggs. The survival data from this study is shown in FIG. 13, while the tracheal and cloacal virus shedding data are shown in FIG. 14A, and 14 B, respectively. The results demonstrate that vaccination using the specified, adjuvanted DNA constructs fully protects chickens against lethal H5N1 challenge.

### Example 11

This example demonstrates the ability of DNA constructs of the present invention to generate a protective immune response in horses. All procedures used in this study were approved by the Institutional Animal Care and Use Committees of the University of Kentucky (Lexington, Ky.) and the Vaccine Research Center, NIAID, NIH (Bethesda, Md.). Animals were maintained according to the animal care guidelines of the Maxwell H. Gluck Equine Research Foundation and were housed from birth at the Veterinary Science Research Farm of the University of Kentucky. Prior to the start of the study, baseline blood chemistry, CBC, other health data and immunologic parameters were determined and ponies of both sexes (1-4 years of age) were randomly assigned to experimental groups. All ponies were seronegative for detectable antibody to equine influenza virus and had no history of signs of influenza infection prior to the start of the study. Ponies were held in individual isolation stalls during immunizations and were group-housed in pastures between immunizations. Animals were identified by microchip.

### Immunogen and Plasmid Construction

Plasmids encoding HA from A/equine/Ohio/1/2003 (H3N8) (GenBank #ABA39846) and A/equine/Bari/2005 (H3N8) (GenBank #ABM47075) were synthesized by GeneArt (Regensburg, Germany). Amino acids 1-345 of A/equine/Aboyne/1/2005 (H3N8) (GenBank #ABP35601) HA were fused with amino acids 346-565 of A/Hong Kong/1/1992 (H3N8) (GenBank #AAA62470) to generate full-length HA gene as the conserved portion of HA2 of A/equine/Aboyne/1/2005 is not available in the NCBI protein database. All HA genes were synthesized using mammalian preferred codons as described by Kong, W. P. et al. (2006, PNAS 103:15987-15991) and cloned into CMV/R expression vector for efficient expression in mammalian cells.

### Influenza Virus Preparation

Influenza A/equine/Ohio/03 challenge virus was isolated in embryonated eggs from a nasopharyngeal swab taken from a sick horse. Stocks of virus were prepared for use in various assays by inoculating 10 day old chicken embryos with a 1:1000 dilution of virus. Eggs were incubated at 37° C. for 48 hr and then transferred to 4° C. for 24 hr. Allantoic fluids were harvested and virus content titrated by hemagglutinin (HA), Egg Infectious Dose (EID₅₀), or Tissue Culture Infectious Dose (TCID₅₀) assays as needed.

### Experimental Design

Male and female ponies were randomly assigned to one of three groups consisting of two animals each. Each animal was vaccinated using a vaccine comprising 4 mg of the specified DNA construct(s) in 1 ml PBS, followed by a boost with the same vaccine at four weeks and eight weeks after the initial vaccination. Animals in Group 1 were immunized with a monovalent vaccine comprising the DNA construct CMV/R-Influenza A/equine/Ohio/1/03 (H3N8) HA wt (SEQ ID NO:35), which expresses the HA gene of influenza A/Equine/Ohio/03. Group 2 ponies were immunized with a trivalent vaccine comprising a mixture of the following DNA constructs: CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37). The trivalent DNA vaccine contained 1.33 mg of each of the 3 DNA constructs for a total of 4 mg of DNA. Group 3 control animals did not receive any vaccination. All injections were given intramuscularly (IM) in the pectoral muscle using a conventional needle and syringe. The design of this study is summarized in Table 10.

TABLE 10
Equine Immunogenicity Study Design

| | No. of | | | | | | | Challenge |
|---|---|---|---|---|---|---|---|---|
| Group | Animals | DNA in Vaccine | Dose | Route | Week 0 | Week 4 | Week 8 | Virus |
| 1 | 2 | CMV/R-HA- | 4 mg | IM (needle) | DNA | DNA | DNA | A/Equine/ |
| | | (Ohio/03) | | | | | | Ohio/03 |
| | | (SEQ ID NO: 35) | | | | | | (H3N8) |
| 2 | 2 | CMV/R-HA- | 4 mg | IM (needle) | DNA | DNA | DNA | A/Equine/ |
| | | (Ohio/03)(SEQ ID | | | | | | Ohio/03 |
| | | NO: 35) + CMV/R- | | | | | | (H3N8) |
| | | HA-(Aboyne/05) | | | | | | |
| | | (SEQ ID NO: 39) + | | | | | | |
| | | CMV/R-HA-(Bari-05) | | | | | | |
| | | (SEQ ID NO: 37) | | | | | | |
| 3 | 2 | Not Immunized | N/A | N/A | — | — | — | A/Equine/ |
| | | | | | | | | Ohio/03 |
| | | | | | | | | (H3N8) |

For Groups 1 and 2, 0.5 mL of vaccine was administered at two separate injection sites, one in each pectoral muscle, for a total volume of 1 mL.

### Challenge

Five weeks following the last boost, vaccinated and control ponies were challenged with wild-type Influenza A/equine/Ohio/03 H3N8 virus as described by Chambers T. M. (2009, Equine Vet. Journal 41:87-92). Challenge virus (2×10⁸ egg infectious dose (EID₅₀) units in 7 ml allantoic fluid plus 25 ml PBS) was nebulized using a DeVillbis Ultra-Neb 99 nebulizer, and pumped into a 21.5 m³ tented stall where it was inhaled for 45 minutes (Townsend H. G., 2001 Equine Vet. Journal 33:637-643; Mumford, J. A., et al., 1990 Equine Vet. Journal 22:93-98). Each such group of ponies included both vaccinates and controls. The dose administered was equivalent to approximately 10⁶ EID₅₀ per pony, or per cubic meter of tented stall volume. This dose reliably induces typical clinical signs of disease and active virus shedding for 5-8 days post-infection. Blood was collected periodically throughout the experiment, and the sera tested for the presence of hemagglutination inhibiting antibodies to the Ohio/03 and Aboyne/05 components of the multivalent vaccine, as well as heterologous H3N8 strain A/eq/Richmond/2007 using previously described methods (OIE Manual of Diagnostic Tests and Vaccines for Terrestrial Animals. 2004 Equine Influenza, p. 686-697. Office of International des Epizooties, Paris). The results of this assay, which are shown in FIG. 15, demonstrate that both vaccines induced antibodies that inhibit virus-induced hemagglutination of blood cells.

Sera collected from the animals in this study were also tested for the presence of neutralizing antibodies. Briefly, sera were serially diluted 2-fold using infection medium (Medium 199 (GIBCO)+1 μg/ml TPCK Trypsin) and virus added to the diluted sera to yield final concentrations of 200 TCID50 units of virus per well. Serum-virus mixtures were incubated at room temperature for 60 min and then added to washed MDCK cells, with each serum dilution plated in quadruplicate. Media controls (no virus), and virus controls (no serum) were included on each plate. Plates were incubated at 37° C. for 2 hr, then washed twice with PBS, filled with infection medium, and incubated for a further 48 hr. Results were read by both HA assay of supernatant from each well, and also staining wells with crystal violet to visualize lysis of the monolayers. SNT titers were calculated as 50% endpoints for the serum dilution giving complete inhibition of virus growth. The results of this assay, which are shown FIG. 16, demonstrate that both the monovalent and trivalent vaccine induced the production of antibodies capable of neutralizing H3N8 influenza virus.

### Example 12

This example compares the efficacy of vaccinating horses with DNA constructs of the present invention, using either a needle and syringe or a needle-free system. Vaccines used in this study were prepared as described in Example 11. Similarly, influenza virus used in this study was prepared and titered as described in Example 11. This study used four groups of ponies, each group consisting of four animals. Animals in the first two groups were immunized with a monovalent vaccine comprising the DNA construct CMV/R-Influenza A/equine/Ohio/1/03 (H3N8) HA wt (SEQ ID NO:35) expressing the HA gene of A/Equine/Ohio/03. Delivery of the vaccine was accomplished using either a conventional needle and syringe (Group 1) or a needle-free device (Group 2). Group 3 ponies were immunized with a trivalent vaccine comprising a mixture of the following DNA constructs: CMV/R-Influenza A/equine/Ohio/1/03 (H3N8) HA wt (SEQ ID NO:35), CMV/R-Influenza A/equine/Aboyne/1/05 (H3N8) Ha/h (SEQ ID NO:39), and CMV/R-Influenza A/equine/Bari/2005 (H3N8) HA wt (SEQ ID NO:37) using a needle-free device. The trivalent vaccine contained 1.33 mg of each of the 3 DNA constructs for a total of 4 mg of DNA. Group 4 control animals received vector DNA (CMV/R; SEQ ID NO:41) using a needle-free device at the same total dose and volume as the experimental groups. All injections were given intramuscularly (IM) in the trapezius muscle via conventional needle and syringe or a needle-free device, the PharmaJet® (PharmaJet®, PharmaJet, Inc., Golden, Colo.). The PharmaJet® 0.5 ml SC/IM Injection System was used in accordance with manufacturer's instructions. For all groups, 0.5 mL of vaccine was administered at two separate injection sites for a total volume of 1 mL. Each of the two injection sites were on the same side of the shaved lateral neck approximately 3 inches apart. Each site was marked with a permanent marker to observe reactogenicity, if any. Following injection, the site was monitored at 24 and 48 hours for adverse reactions. The design of this experiment is summarized in Table 11.

TABLE 11
Needle and syringe vs. needle-free system study design

| | No. of | | | Route/Injection | Challenge |
|---|---|---|---|---|---|
| Group | Animals | DNA in Vaccine | Dose | Method | Virus |
| 1 | 4 | CMV/R-HA-(Ohio/03) | 4 mg | IM | A/Equine/ |
| | | (SEQ ID NO: 35) | | (needle/syringe) | Ohio/03 |
| | | | | | (H3N8) |
| 2 | 4 | CMV/R-HA-(Ohio/03) | 4 mg | IM | A/Equine/ |
| | | (SEQ ID NO: 35) | | (needle-free) | Ohio/03 |
| | | | | | (H3N8) |
| 3 | 4 | CMV/R-HA-(Ohio/03) (SEQ ID | 4 mg | IM | A/Equine/ |
| | | NO: 35) + CMV/R-HA-(Bari/05) (SEQ | | (needle-free) | Ohio/03 |
| | | ID NO: 37) + CMV/R-HA-(Aboyne/05) | | | (H3N8) |
| | | (SEQ ID NO: 39) | | | |
| 4 | 4 | CMV/R (SEQ ID NO: 41) | 4 mg | IM | A/Equine/ |
| | | | | (needle-free) | Ohio/03 |
| | | | | | (H3N8) |

One of the ponies sustained a cecocolic intussusception with peritonitis not attributed to vaccination and was humanely euthanized prior to challenge.

### Challenge and Clinical Monitoring

At 15 weeks post-vaccination, animals were challenged with wild-type Influenza A/equine/Ohio/03 H3N8 virus as described in Example 11. To assess clinical protection following experimental challenge with virus, physical examinations were performed on all horses daily from 1 day before (Day −1) to 8 days after (Day +8) virus administration. Examinations included measurements of rectal temperature (FIG. 17), respiratory rate, auscultation of lung sounds, palpation of submandibular and parotid lymph nodes, general demeanor, and presentation of nasal discharge or spontaneous coughing. A clinical scoring system (Shown below in Table 12) was used which was similar to that previously described by Chamber T. M. (Equine Vet. Journal 33:630-636). Examinations and clinical scoring were done by a veterinarian blinded to the vaccination status of the horses.

TABLE 12
Clinical signs scoring index

| Clinical Sign | Degree | Score |
|---|---|---|
| Coughing | No coughing | 0 |
| | Coughed once | 1 |
| | Coughed twice or more | 2 |
| Nasal discharge | No discharge | 0 |
| | Abnormal serous | 1 |
| | Abnormal mucopurulent | 2 |
| | Abnormal profuse | 3 |
| Respiration | Normal <36/min | 0 |
| | Abnormal (dyspnea/tachypnea) >36/min | 1 |
| Demeanor | No depression | 0 |
| | Depression present (lethargy, inappetence) | 1 |
| Lymph nodes | Normal | 0 |
| | Enlarged | 1 |
| | Draining | 2 |

Σ quadrant scores/10, rounded to nearest whole number

| | | |
|---|---|---|
| Lung Score | Normal | 0 |
| | Slight insp wheeze | 1 |
| | Strong insp wheeze | 2 |
| | Insp/exp wheeze | 3 |
| | Crackles | 4 |
| | Pleuropneumonia | 5 |

### Serological Analysis

Venous blood samples were collected via jugular venipuncture prior to the first vaccination and weekly or bi-weekly thereafter. Other blood samples were drawn on Days 0, 7, 14, and 21 post-challenge for serological analyses. Immediately prior to blood collection, the area over the jugular vein was shaved, wiped clean with alcohol, and allowed to dry. All sera were tested for the presence of antibody to influenza A H3N8 strains A/eq/Ohio/03, A/eq/Richmond/1/07, and A/eq/Aboyne/05 (all American lineage) using the single radial hemolysis (SRH) (OIE Manual of Diagnostic Tests and Vaccines for Terrestrial Animals. 2004 Equine Influenza, p. 686-697. Office of International des Epizooties, Paris; Wood, J. M. et al., 1983 J. Hyg. (Loud) 90:371-384). For this analysis, sheep erythrocytes were used and zones of hemolysis were measured after a 20 hr incubation period. All sera were tested for non-specific lysis. SRH antibody levels were expressed as the area of hemolysis (mm²). The results of these assays, which are shown in FIG. 18, demonstrate that both the monovalent and trivalent vaccines are capable of eliciting antibody responses against all three strains of influenza that were tested, and that vaccination using a needle-free system results in higher titers of such antibody.

The sera obtained from the horses were also tested for anti-influenza antibodies using the previously described hemagglutination-inhibition (HI) assay (OIE Manual of Diagnostic Tests and Vaccines for Terrestrial Animals. 2004 Equine Influenza, p. 686-697. Office of International des Epizooties, Paris). For HI analysis, all sera were pre-treated with trypsin-periodate and assays were done using viruses diluted to 1:8 and 0.5% chicken erythrocytes. The results of these assays, which are shown in FIG. 19, demonstrate that both the monovalent and trivalent vaccines elicit antibodies against influenza A/Equine/Ohio/03 and influenza A/Equine/Richmond/1/07. The results also demonstrate that vaccination using a needle-free system results in higher titers of anti-viral antibodies.

### Viral Shedding and Quantitation

Nasopharyngeal swabs were collected on the day prior to challenge, and daily for 8 days post challenge. Dacron swabs were inserted 10-15 cm up the nasal meatus, immersed in 1 ml of PBS/5% glycerol/1% antibiotic solution and stored at 4° C. until testing. Viral shedding was detected by inoculation into embryonated hen eggs followed by 3 days incubation at 35° C. and testing by hemagglutination assay (OIE Manual of Diagnostic Tests and Vaccines for Terrestrial Animals. 2004 Equine Influenza, p. 686-697. Office of International des Epizooties, Paris; OIE Manual of Diagnostic Tests and Vaccines for Terrestrial Animals. 2000 Equine Influenza. Office of International des Epizooties, World Organization for Animal Health, Paris). Virus RNA content was quantified by quantitative RT-PCR (qRT-PCR) (Lu, Z. T., et al., 2009 J. Clin. Microbiol. 47:3907-3913). One step real time RT-PCR was performed using TaqMan rRT-PCR, using in vitro-transcribed (IVT) RNA as a standard. The IVT-RNA concentration was determined mathematically and the genome copy number was calculated (Lu, Z. et al., 2009 J. Clin. Microbiol. 47:3907-3913). Viral loads as determined by RT-PCR are shown in FIG. 20.

### Inflammatory Cytokine Response Post-Challenge

To measure the levels of inflammatory cytokines, venous blood samples were taken on the day prior to challenge, and then daily for 6 days post-challenge. The samples were collected into PAXgene (Qiagen, Valencia, Calif.) tubes, which contain a stabilizing additive to preserve the RNA expression profile (Rainen, L., et al. 2002. Clin. Chem. 48:1883-1890). RNA was isolated from PAXgene blood as per manufacturer's recommendations and quantified by OD₂₆₀. Quantitative RT-PCR was carried out on the Applied Biosystems 7900 HT Fast Real-Time PCR System and data were analyzed as a relative quantification study as previously described (Livak, K. J., et al., 2001. CT Method. Methods 25; 402-408; Chambers, T. M., et al., 2009. Equine Vet. Journal 41:87-92). The sample taken prior to challenge (Day −1) was chosen as the calibrator sample. Relative quantification was then used to compare gene expression levels post-challenge to Day −1. Equine β-glucuronidase (β-GUS) was used as an endogenous control to normalize for differences in RNA and cDNA (Aerts, J., et al. 2004 Biotechniques 36:84-86, 88, 90; Dheda, K, et al., 2004. Biotechniques 37:112-114, 116, 118; Shipley, J. M., 1991. Genomics 10:1009-1018). Reverse transcription conditions and FAM-labeled primer probes for IFNγ, IL-1β, IL-6, TNF-α and β-GUS were as previously described (Dheda, K, et al., 2004. Biotechniques 37:112-114, 116, 118). The 2^{-ΔΔCT} method for analyzing relative gene expression from real-time quantitative PCR experiments was employed for analysis of data (Chambers, T. M., 2009. Equine Vet. Journal 41:87-92; Livak, K. J., et al., 2001. CT Method. Methods 25; 402-408). Each sample was tested in duplicate for each of the cytokine targets and the endogenous control. The results of these analyses are shown in FIG. 21 (interferon-gamma), FIG. 22A (granzyme B), FIG. 22B (interferon gamma), FIG. 23A (interleukin-1), FIG. 23B (interleukin-6), and FIG. 24 (TNF-alpha). The results of these tests indicate that all vaccinated animals were protected against peak inflammatory responses on days 2 and 3 post-challenge compared to controls. In addition, needle-free delivery to the trivalent vaccine results in higher IFN-expression compared to controls.

The results of this study demonstrate that IM injection of DNA constructs of the present invention elicits neutralizing antibodies while reducing inflammatory responses in horses challenged with H3N8 virus. This is true of both monovalent and trivalent vaccine compositions, whether delivered by needle and syringe or by needle-free methods. The results also indicate that use of a needle-free injection system results in the production of a greater immune response as compared to the response resulting from injection using a needle and syringe.

### Example 13

This example compares the efficacy of DNA vaccination of pigs with DNA constructs of the present invention, using either a needle and syringe or a needle-free system. All procedures were approved by and in compliance with the Institutional Animal Care and Use Committees of the National Animal Disease Center, USDA (Ames, Iowa) and the Vaccine Research Center, NIAID, NIH (Bethesda, Md.). One hundred three-week old cross-bred pigs were obtained from a herd free of SIV and porcine reproductive and respiratory syndrome virus (PRRSV) and treated with ceftiofur crystalline free acid according to label (Pfizer Animal Health, New York, N.Y.) to reduce bacterial contaminants prior to the start of the study. Pigs were housed in biosafety level 2 (BSL2) containment during the vaccine phase of the study. On the day of challenge, pigs were transferred to ABSL3 containment for the remainder of the experiment.

### Immunogen and Plasmid Construction

Plasmids encoding HA from A/swine/Ohio/2007 (H1N1) (GenBank #EU604689), A/swine/North Carolina/2008 (H3N2) (GenBank #ACS92895), and A/swine/California/09 were synthesized by GeneArt (Regensburg, Germany). All HA genes were synthesized using mammalian preferred codons (Kong, W. P., 2006. PNAS 103:15987-15991) and cloned into CMV/R expression vector for efficient expression in mammalian cells.

### Immunizations

One hundred pigs were randomly assigned to one of 10 groups, each group consisting of 10 animals. The animals in each group were vaccinated with a prime and 2 homologous boosts 3 and 6 weeks post-prime with 4 mg of the specified DNA construct(s) in 1 ml PBS. All animals were immunized intramuscularly in the post-auricular region of the neck, either with conventional needle and syringe, or by the Pharmajet® needle-free injection device (PHARMAJET®, PharmaJet, Inc., Golden, Colo.). The PHARMAJET® 0.5 ml SC/IM Injection System was used in accordance with manufacturer's instructions. Animals in the first 6 groups were immunized using needle and syringe, with groups 1-3 receiving vector DNA (CMV/R; SEQ ID NO:41) lacking an insert as controls, groups 4 and 5 receiving a trivalent vaccine comprising the DNA constructs CMV/R-Influenza A/swine/Ohio/51145/2007 (H1N1) Ha/h (SEQ ID NO:29), CMV/R-Influenza A/swine/North Carolina/R08-001877/2008 (H3N2) Ha/h (SEQ ID NO:31), and CMV/R-Influenza A/swine/California/04/09 (H1N1) Ha/h (SEQ ID NO:33), and group 6 receiving a monovalent DNA vaccine comprising the DNA construct CMV/R-Influenza A/swine/California/04/09 (H1N1) Ha/h; SEQ ID NO:33), that encodes HA from only A/swine/California/09 H1N1. Groups 7-9 were immunized using the PHARMAJET® needle-free device, with groups 7 and 8 receiving the trivalent DNA vaccine identical to groups 4 and 5, and group 9 receiving the monovalent DNA vaccine identical to group 6. Group 10 received the same trivalent vaccine via needle and syringe. Blood samples were collected 2 weeks after each immunization and immediately prior to challenge. A summary of the study design is listed in Table 13.

TABLE 13
needle and syringe vs needle-free vaccination in pigs study design

| | | | | Route/ | |
|---|---|---|---|---|---|
| Group | # of Animals | DNA in Vaccine | Dose | Injection Method | Challenge virus |
| 1 | 10 | Control (CMV/R; SEQ ID | 4 mg | IM | A/swine/Ohio/2007 |
| | | NO: 41) | | (Needle) | H1N1 |
| 2 | 10 | Control (CMV/R; SEQ ID | 4 mg | IM | A/swine/California/ |
| | | NO: 41) | | (Needle) | 09 H1N1 |
| 3 | 10 | Control (CMV/R; SEQ ID | 4 mg | IM | A/swine/Illinois/ |
| | | NO: 41) | | (Needle) | 09 H3N2 |
| 4 | 10 | H1N1 (SEQ ID | 4 mg | IM | A/swine/Ohio/2007 |
| | | NO: 29) + H3N2 (SEQ ID | | (Needle) | H1N1 |
| | | NO: 31) + CMV/R-HA-Cal- | | | |
| | | 09 (SEQ ID NO: 33) | | | |
| 5 | 10 | H1N1 (SEQ ID | 4 mg | IM | A/swine/California/ |
| | | NO: 29) + H3N2 (SEQ ID | | (Needle) | 09 H1N1 |
| | | NO: 31) + CMV/R-HA-Cal- | | | |
| | | 09 (SEQ ID NO: 33) | | | |
| 6 | 10 | CMV/R-HA-(Cal-09) | 4 mg | IM | A/swine/California/ |
| | | (SEQ ID NO: 33) + CMV/R | | (Needle) | 09 H1N1 |
| | | (SEQ ID NO: 41) | | | |
| 7 | 10 | H1N1 (SEQ ID | 4 mg | Needle free | A/swine/Ohio/2007 |
| | | NO: 29) + H3N2 (SEQ ID | | | H1N1 |
| | | NO: 31) + CMV/R-HA-Cal- | | | |
| | | 09 (SEQ ID NO: 33) | | | |
| 8 | 10 | CMV/R-HA-(Cal-09) | 4 mg | Needle-free | A/swine/California/ |
| | | (SEQ ID NO: 33) + CMV/R | | | 09 H1N1 |
| | | (SEQ ID NO: 41) | | | |
| 9 | 10 | H1N1 (SEQ ID | 4 mg | Needle free | A/swine/California/ |
| | | NO: 29) + H3N2 (SEQ ID | | | 09 H1N1 |
| | | NO: 31) + CMV/R-HA-Cal- | | | |
| | | 09 (SEQ ID NO: 33) | | | |
| 10 | 10 | H1N1 (SEQ ID | 4 mg | IM | A/swine/Illinois/ |
| | | NO: 29) + H3N2 (SEQ ID | | (Needle) | 09 H3N2 |
| | | NO: 31) + CMV/R-HA-Cal- | | | |
| | | 09 (SEQ ID NO: 33) | | | |

### Challenge

Three weeks after the final boost, all pigs were challenged intranasally with 2×10⁶ TCID₅₀/pig of either A/swine/Ohio/2007 H1N1, A/swine/Illinois/09 H3N2, or A/swine/California/09 H1N1. Challenge viruses were prepared in MDCK cells. Prior to intranasal challenge, pigs were anesthetized with an intramuscular injection of a cocktail of ketamine (8 mg/kg), xylazine (4 mg/kg), and Telazol (6 mg/kg, Fort Dodge Animal Health, Fort Dodge, Iowa). Groups 1,4, and 7 were challenged with A/swine/Ohio/2007 H1N1, groups 3 and 10 were challenged with A/swine/Illinois/09 H3N2, and groups 2,5,6,8, and 9 were challenged with A/swine/California/09 H1N1. Pigs were observed daily for clinical signs of disease. Nasal swabs were taken and placed in 2 ml minimal essential medium (MEM) on 0, 3, 5, and 7 dpi or days post contact (dpc) to evaluate nasal shedding. Five pigs/group were humanely euthanized with a lethal dose of pentobarbital (Sleepaway, Fort Dodge Animal Health, Fort Dodge, Iowa) on day 5 post-challenge to evaluate lung lesions and viral load in the lung. The remaining challenged pigs were euthanized on day 12. Blood samples were taken immediately prior to euthanasia.

### Hemagglutination Inhibition (HI) Assay

Pig serum was collected 2 weeks after each immunization, immediately prior to challenge, and immediately prior to euthanasia at 5 days, and 12 days post challenge. For each of these time points, a hemagglutination inhibition assay was performed against homologous virus strains to assess antibody responses to vaccine treatments as previously described (Vincent, A. L., 2010. Vaccine 28:2782-2787). Briefly, sera were heat inactivated at 56° C. for 30 min, then treated to remove non-specific hemagglutinin inhibitors and natural serum agglutinins by treatment with a 20% suspension of kaolin (Sigma Aldrich, St. Louis, Mo.) and adsorption with 0.5% turkey red blood cells (RBC). The HI assays were then performed using virus strains homologous to the challenge strain for each group. An additional HI assay was performed on sera collected prior to challenge against all three challenge strains to measure heterologous antibody responses. The results of this study, which are shown in FIG. 25, indicate that all vaccinated animals produced a homologous antibody response. They also demonstrate that infection with challenge virus results in a robust anamnestic response in vaccinated groups, indicating adaptive memory conferred by vaccines comprising DNA constructs of the present invention (shown in FIG. 25). Furthermore, as HI assay using a heterologous virus indicates that the monovalent vaccine elicits heterologous antibody responses against other H1N1 strains, while the trivalent vaccine elicits heterosubtypic antibody responses against both H1N1 and H3N2 influenza strains (shown in FIG. 26)

### Viral Shedding and Quantitation

Nasal swabs (Fisherbrand Dacron swabs, Fisher Scientific, Pittsburgh, Pa.) were taken on 0, 4, 6, 8, and 12 days post-infection (dpi) to evaluate nasal virus shedding by dipping the swab in minimal essential medium (MEM) and inserting the swab approximately 2.5 cm into each nares. The presence of swine virus was detected using a real-time PCR assay developed to detect avian influenza A viruses (Spackman, E., et al., 2008. Methods Mol. Biol. 436:19-26), but modified to detect the pandemic H1N1 virus. (Lorusso, A., et al., 2010. J. Virol. Methods 164:83-87). The result of this analysis is shown in FIG. 27.

Although the invention has been described with reference to embodiments and examples, it should be understood that various modifications can be made without departing from the spirit of the invention. Accordingly, the invention is limited only by the following claims. All references cited herein are hereby expressly incorporated by reference.

1. An influenza immunogen comprising one or more DNA constructs encoding at least two divergent influenza HAs, wherein each of said one or more DNA constructs encodes one or more of said at least two divergent influenza HAs, wherein an immune response is induced to a plurality of strains of influenza virus upon administration of said influenza immunogen to a subject, wherein at least one strain of said plurality of strains does not encode any of said divergent influenza HAs.

2. The influenza immunogen of claim 1, wherein said at least two divergent influenza HAs are selected from the group consisting of influenza H1 HAs, influenza H3 HAs, and a mixture of H1 and H3 influenza HAs.

3. The influenza immunogen of claim 1, wherein-said one or more DNA constructs encode at least three divergent influenza HAs selected from the group consisting of influenza H1 HAs, influenza H3 HAs, and a mixture of H1 and H3 influenza HAs.

4. The influenza immunogen of claim 1, wherein at least one of said one or more DNA constructs encode H3 HA from Influenza A/equine/Bari/2005, Influenza A/equine/Ohio/1/2003, or Influenza A/equine/Aboyne/1/2005.

5. The influenza immunogen of claim 1, wherein said one or more DNA constructs encode H3 HA from Influenza A/equine/Bari/2005, Influenza A/equine/Ohio/1/2003, and Influenza A/equine/Aboyne/1/2005.

6. The influenza immunogen of claim 1, wherein at least one of said one or more DNA constructs encodes H1 HA from Influenza A/swine/California/04/2009 or Influenza A/swine/Ohio/51145/2007.

7. The influenza immunogen of claim 1, wherein at least one of said one or more DNA constructs encodes H3 HA from Influenza A/swine/North Carolina/R08-001877/2008.

8. The influenza immunogen of claim 1, wherein said one or more DNA constructs encode H1 HA from Influenza A/swine/California/04/2009, H1 HA from Influenza A/swine/Ohio/51145/2007, and H3 HA from Influenza A/swine/North Carolina/R08-001877/2008.

9. The influenza immunogen of claim 1, wherein at least one of said DNA constructs encodes an influenza HA having an amino acid sequence selected from the group consisting of SEQ ID NO:30, SEQ ID NO:32, SEQ ID NO:34, SEQ ID NO:36, SEQ ID NO:38, and SEQ ID NO:40.

10. The influenza immunogen of claim 1, wherein at least one of said DNA constructs comprises a nucleic acid sequence selected from the group consisting of SEQ ID NO:29, SEQ ID NO:31, SEQ ID NO:33, SEQ ID NO:35, SEQ ID NO:37, and SEQ ID NO:39.

11. The influenza immunogen of claim 1, wherein said at least one strain of said plurality of strains comprises an influenza virus having an H1 HA or an H3 HA.

12. A method of inducing an immune response to a plurality of strains of influenza virus in a subject in need thereof, said method comprising administering to said subject one or more DNA constructs encoding at least two divergent influenza HAs, wherein each of said one or more DNA constructs encodes one or more of said at least two divergent influenza HAs, wherein said immune response to the plurality of strains of influenza virus is induced upon said administration to said subject, and wherein at least one strain of said plurality of strains does not encode any of said influenza HAs.

13. The method of claim 12, wherein said at least two divergent influenza HAs are selected from the group consisting of influenza H1 HAs, influenza H3 HAs, and a mixture of H1 and H3 influenza HAs.

14. The method of claim 12, wherein said one or more DNA constructs encode at least three divergent influenza HAs selected from the group consisting of influenza H1 HAs, influenza H3 HAs, and a mixture of H1 and H3 influenza HAs.

15. The method of claim 12, wherein at least one of said one or more DNA constructs encodes H3 HA from Influenza A/equine/Bari/2005, Influenza A/equine/Ohio/1/2003, or Influenza A/equine/Aboyne/1/2005.

16. The method of claim 12, wherein said one or more DNA constructs encode H3 HA from Influenza A/equine/Bari/2005, Influenza A/equine/Ohio/1/2003, and Influenza A/equine/Aboyne/1/2005.

17. The method of claim 12, wherein at least one of said one or more DNA constructs encodes H1 HA from Influenza A/swine/California/04/2009 or Influenza A/swine/Ohio/51145/2007.

18. The method of claim 12, wherein at least one of said one or more DNA constructs encodes H3 HA from Influenza A/swine/North Carolina/R08-001877/2008.

19. The method of claim 12, wherein said one or more DNA constructs encode H1 HA from Influenza A/swine/California/04/2009, H1 HA from Influenza A/swine/Ohio/51145/2007, and H3 HA from Influenza A/swine/North Carolina/R08-001877/2008.

20. The method of claim 12, wherein at least one of said one or more DNA constructs encodes an influenza HA having an amino acid sequence selected from the group consisting of SEQ ID NO:30, SEQ ID NO:32, SEQ ID NO:34, SEQ ID NO:36, SEQ ID NO:38, and SEQ ID NO:40.

21. The method of claim 12, wherein at least one of said one or more DNA constructs comprises a nucleic acid sequence selected from the group consisting of SEQ ID NO:29, SEQ ID NO:31, SEQ ID NO:33, SEQ ID NO:35, SEQ ID NO:37, and SEQ ID NO:39.

22. The method of claim 12, wherein said at least one strain of said plurality of strains comprises an influenza virus having an H1 HA or an H3 HA.

23. The method of claim 22, wherein said influenza virus is an equine influenza virus or a swine influenza virus.

24. The method of claim 12, wherein said subject is selected from the group consisting of a human, horse, pig, bird, cat, and dog.

25. The method of claim 12, wherein said one or more DNA constructs are administered by a route selected from the group consisting of topical, intranasal, intraocular, subcutaneous, transdermal, intradermal, intramuscular, parenteral, gastrointestinal, transbronchial, and transalveolar routes.

26. The method of claim 25, wherein said route is intramuscular.

27. The method of claim 25, wherein said one or more DNA constructs are administered by a needle-free injector.

28. The method of claim 12, wherein said one or more DNA constructs are administered in a formulation comprising an adjuvant.

29. A DNA construct comprising a nucleic acid sequence selected from the group consisting of SEQ ID NO:41, SEQ ID NO:42, SEQ ID NO:43, SEQ ID NO:44, SEQ ID NO:45, SEQ ID NO:46, SEQ ID NO:29, SEQ ID NO:31, SEQ ID NO:33, SEQ ID NO:35, SEQ ID NO:37, and SEQ ID NO:39.

30. The DNA construct of claim 29, wherein said DNA construct comprises a nucleic acid sequence selected from the group consisting of SEQ ID NO:29, SEQ ID NO:31, SEQ ID NO:33, SEQ ID NO:35, SEQ ID NO:37, and SEQ ID NO:39.


SEQUENCE LISTING (47 sequences)

SEQ ID NO: 1 - protein, 571 aa, Influenza virus
ICQMEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 2 - protein, 565 aa, Influenza virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCR

SEQ ID NO: 3 - protein, 568 aa, Influenza virus
MEKTVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 4 - protein, 568 aa, Influenza virus
MEKIVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRISHFEKIQIIPKSSWSNHDASSGVSSACPYLGKSSFFRNVVWLIKKNSTYPTIKRSYNNTNQEDLLVLWGIHHPNDAAEQTKLYQNPTTYISVGTSTLNQRLVPEIATRPKVNGQSGRIEFFWTILKPNDAINFESNGNFIAPEYAYKIVKKGDSTIMKSELEYGNCNTKCQTPMGAINSSMPFHNIHPLTIGECPKYVKSNRLVLATGLRNAPQRERRRKKRGLFGAIAGFIEGGWQGMVDGWYGYHHSNEQGSGYAADQESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSLYSTVASSLALAIMVAGLSLWMCSNGSLQCRICI

SEQ ID NO: 5 - protein, 567 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPPNDLCYPGNFNDYEELKHLLSRINHFEKIQIIPKSSWSDHEASSGVSSACPYQGRSSFFRNVVWLIKKNSAYPTIKRSYNNTNQEDLLVLWGIHHPNDAAEQTRLYQNPTTYISVGTSTLNQRLVPKIATRSKVNGQSGRMEFFWTILKPNDAISFESNGNFIAPEYAYKIVKKGDSAIMKSELEYGNCNTKCQTPMGAINSSMPFHNIHPLTIGECPKYVKSSRLVLATGLRNSPQREKRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECIESVRNGTYGYPQYSEEARLKREEISGVKLESIGTYQILSIYSTVASSLALAIMVAGLSLWMCSNGSLQCRICI

SEQ ID NO: 6 - protein, 568 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 7 - protein, 568 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASAGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSSRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 8 - protein, 546 aa, Influenza virus
ICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKSNDAINFESNGNFIAPENAYKIVKKGDSTIMKSELEYGNCNTKCQTPIGAINSSMPFHNIHPLTIGECPKYVKSNRLILATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLFLWMCSNGSLQC

SEQ ID NO: 9 - protein, 556 aa, Influenza virus
VLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNG

SEQ ID NO: 10 - protein, 549 aa, Influenza virus
DQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPSDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCK

SEQ ID NO: 11 - protein, 567 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIVKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 12 - protein, 11 aa, Artificial Sequence
  Influenza non-cleavage site
PQRERRRKKRG

SEQ ID NO: 13 - protein, 7 aa, Artificial Sequence
  Cleavage site
PQRETRG

SEQ ID NO: 14 - DNA, 6121 bp, Artificial Sequence
  CMV/R Influenza A/Anhui/1/2005 (H5N1) HA mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccgccaacgatctgtgttaccccggcaacttcaacgattacgaggaactgaagcacctgctgtcccggatcaaccacttcgagaagatccagatcatccccaagtcctcttggagcgatcacgaagcctctagcggagtgtctagcgcctgtccttaccagggcactcccagcttcttcagaaacgtggtgtggctgatcaagaagaacaacacctaccccaccatcaagagaagctacaacaacaccaaccaggaagatctgctgatcctgtggggaatccaccacagcaatgatgccgccgagcagaccaagctgtaccagaaccccaccacctatatcagcgtgggcaccagcaccctgaatcagagactggtgcccaagatcgccaccagatccaaggtgaacggccagagcggcaggatggacttcttctggaccatcctgaagcccaacgacgccatcaacttcgagagcaacggcaactttatcgcccctgagtacgcctacaagatcgtgaagaagggcgacagcgccatcgtgaagagcgaggtggaatacggcaactgcaacaccaagtgccagacacctatcggcgccatcaacagcagcatgcccttccacaacatccaccctctgaccatcggcgagtgccctaagtacgtgaagagcaacaagctggtgctggccacaggcctgagaaatagccccctgcgggagaccaggggcctgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcgtccggaacggcacctacgactaccctcagtacagcgaggaagccaggctgaagagggaagagatcagcggcgtgaaactggaatccatcggcacctaccagatcctgagcatctacagcacagtggcctcttctctggccctggccattatggtcgccggactgagcctgtggatgtgcagcaatggcagcctgcagtgcaggatctgcatctgatgaacacgtggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 6123 bp, Artificial Sequence
  CMV/R Influenza A/Vietnam/1203/2004 (H5N1) HA mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagaaacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagggcaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 6122 bp, Artificial Sequence
  CMV/R Influenza A/Nigeria/641/2006 (H5N1) HA mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccattgtgagcctggtgaagagcgaccagatctgcattggctaccacgccaacaatagcacagagcaggtggacaccatcatggagaaaaacgtgaccgtgacccacgcccaggatattctggagaaaacccacaacggcaagctgtgtgatctggacggcgtgaagcctctgatcctgagagattgtagcgtggccggatggctgctgggaaaccccatgtgcgacgagtttctgaacgtgcccgagtggagctatatcgtggagaagatcaaccccgccaacgatctgtgttaccccggcaacttcaacgattacgaggagctgaagcacctgctgtcccggatcaaccacttcgagaagatccagatcatccccaagagcagctggagcgatcacgaagccagctctggagtgtctagcgcctgtccttatcagggccggagcagcttcttcagaaacgttgtgtggctgatcaagaaggacaacgcctaccccaccatcaagcggagctacaacaacaccaaccaggaggatctgctggtcctgtggggaatccaccaccctaatgatgccgccgagcagaccagactgtaccagaaccccaccacctacatctctgtgggcaccagcaccctgaatcagagactggtgcccaagatcgccaccagatccaaggtgaacggccagagcggcagaatggagttcttctggaccatcctgaagcccaacgacgccatcaacttcgagagcaacggcaatttcatcgcccccgagaacgcctataagatcgtgaagaagggcgacagcaccatcatgaagtccgagctggagtacggcaactgcaacaccaagtgccagacacctatcggcgccatcaacagcagcatgcccttccacaacatccaccctctgaccatcggcgagtgccctaagtacgtgaagagcaacagactggtgctggccacaggcctgagaaatagccctcagagagagaccagaggcctgtttggagccatcgccggctttatcgaagggggatggcagggaatggtcgatggctggtacggctaccaccacagcaatgagcagggctctggatatgccgccgataaggagtctacccagaaggccatcgacggcgtcaccaacaaggtgaacagcatcatcgacaagatgaacacccagtttgaggctgtgggcagggagttcaacaacctggagcggaggatcgagaacctgaacaagaagatggaggacggcttcctggatgtgtggacctacaatgccgaactgctggtgctgatggagaacgagaggaccctggacttccacgacagcaacgtgaagaacctgtacgacaaagtgcggctgcagctgagagataacgccaaggagctgggcaacggctgcttcgagttctaccacagatgcgacaacgagtgcatggagtctgtgaggaacggcacctacgactaccctcagtatagcgaggaggccaggctgaagagagaggagatcagcggcgtcaaactggagagcatcggcacctatcagatcctgagcatctacagcacagtggcctcttctctggccctggccattatggtggccggactgagcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 1705 bp, Artificial Sequence
  Human codon optimized Influenza HA H5 A-Indonesia
atggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgagcagaccagactgtaccagaaccccaccacctatatcagcatcggcaccagcaccctgaatcagagactggtgcccaagatcgccaccagatccaaggtgaacggccagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctgatgaacacgtg

SEQ ID NO: 18 - DNA, 1692 bp, Artificial Sequence
  Chicken codon optimized Influenza HA H5 A-Indonesia
atggagaagatcgtgctgctgctggccatcgtgagcctggtgaagagcgatcagatctgcatcggctaccacgccaacaacagcaccgagcaggtggataccatcatggagaagaacgtgaccgtgacccacgcccaggatatcctggagaagacccacaacggcaagctgtgcgatctggatggcgtgaagcccctgatcctgagagattgcagcgtggccggctggctgctgggcaaccccatgtgcgatgagttcatcaacgtgcccgagtggagctacatcgtggagaaggccaaccccaccaacgatctgtgctaccccggcagcttcaacgattacgaggagctgaagcacctgctgagcagaatcaaccacttcgagaagatccagatcatccccaagagcagctggagcgatcacgaggccagcagcggcgtgagcagcgcctgcccctacctgggcagccccagcttcttcagaaacgtggtgtggctgatcaagaagaacagcacctaccccaccatcaagaagagctacaacaacaccaaccaggaggatctgctggtgctgtggggcatccaccaccccaacgatgccgccgagcagaccagactgtaccagaaccccaccacctacatcagcatcggcaccagcaccctgaaccagagactggtgcccaagatcgccaccagaagcaaggtgaacggccagagcggcagaatggagttcttctggaccatcctgaagcccaacgatgccatcaacttcgagagcaacggcaacttcatcgcccccgagtacgcctacaagatcgtgaagaagggcgatagcgccatcatgaagagcgagctggagtacggcaactgcaacaccaagtgccagacccccatgggcgccatcaacagcagcatgcccttccacaacatccaccccctgaccatcggcgagtgccccaagtacgtgaagagcaacagactggtgctggccaccggcctgagaaacagcccccagagagagaccagaggcctgttcggcgccatcgccggcttcatcgagggcggctggcagggcatggtggatggctggtacggctaccaccacagcaacgagcagggcagcggctacgccgccgataaggagagcacccagaaggccatcgatggcgtgaccaacaaggtgaacagcatcatcgataagatgaacacccagttcgaggccgtgggcagagagttcaacaacctggagagaagaatcgagaacctgaacaagaagatggaggatggcttcctggatgtgtggacctacaacgccgagctgctggtgctgatggagaacgagagaaccctggatttccacgatagcaacgtgaagaacctgtacgataaggtgagactgcagctgagagataacgccaaggagctgggcaacggctgcttcgagttctaccacaagtgcgataacgagtgcatggagagcatcagaaacggcacctacaactacccccagtacagcgaggaggccagactgaagagagaggagatcagcggcgtgaagctggagagcatcggcacctaccagatcctgagcatctacagcaccgtggccagcagcctggccctggccatcatgatggccggcctgagcctgtggatgtgcagcaacggcagcctgcagtgcagaatctgcatc

SEQ ID NO: 19 - DNA, 7907 bp, Artificial Sequence
  Synthetic Adenovirus-5 construct with HA H5 A-Indonesia insert
ttaattaaccgcaattctcatgtttgacagcttatcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtaccagtgcactggcctagagcggccccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgtcaccgtcgtcgacacgtgtgatcagatatcgcggccgctctagagatatcgccaccatggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgagcagaccagactgtaccagaaccccaccacctatatcagcatcggcaccagcaccctgaatcagagactggtgcccaagatcgccaccagatccaaggtgaacggccagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctgatgaacacgtggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccagggccgcataacttcgtataatgtatgctatacgaagttataagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtcgatcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattgtt

SEQ ID NO: 20 - DNA, 6120 bp, Artificial Sequence
  CMV/R Influenza A/Turkey/turkey (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccattgtgagcctggtgaagagcgaccagatctgcattggctaccacgccaacaatagcacagagcaggtggacaccatcatggagaaaaacgtgaccgtgacccacgcccaggatattctggagaaaacccacaacggcaagctgtgtgatctggacggcgtgaagcctctgatcctgagagattgtagcgtggccggatggctgctgggaaaccccatgtgcgacgagtttctgaacgtgcccgagtggagctatatcgtggagaagatcaaccccgccaacgatctgtgttaccccggcaacttcaacgattacgaggagctgaagcacctgctgtcccggatcaaccacttcgagaagatccagatcatccccaagagcagctggagcgatcacgaagccagcgccggagtgtctagcgcctgtccttatcagggccggagcagcttcttcagaaacgttgtgtggctgatcaagaaggacaacgcctaccccaccatcaagcggagctacaacaacaccaaccaggaggatctgctggtcctgtggggaatccaccaccctaatgatgccgccgagcagaccagactgtaccagaaccccaccacctacatctctgtgggcaccagcaccctgaatcagagactggtgcccaagatcgccaccagatccaaggtgaacggccagagcggcagaatggagttcttctggaccatcctgaagcccaacgacgccatcaacttcgagagcaacggcaatttcatcgcccccgagaacgcctataagatcgtgaagaagggcgacagcaccatcatgaagtccgagctggagtacggcaactgcaacaccaagtgccagacacctatcggcgccatcaacagcagcatgcccttccacaacatccaccctctgaccatcggcgagtgccctaagtacgtgaagagcagcagactggtgctggccacaggcctgagaaatagccctcagagagagaccagaggcctgtttggagccatcgccggctttatcgaagggggatggcagggaatggtcgatggctggtacggctaccaccacagcaatgagcagggctctggatatgccgccgataaggagtctacccagaaggccatcgacggcgtcaccaacaaggtgaacagcatcatcgacaagatgaacacccagtttgaggctgtgggcagggagttcaacaacctggagcggaggatcgagaacctgaacaagaagatggaggacggcttcctggatgtgtggacctacaatgccgaactgctggtgctgatggagaacgagaggaccctggacttccacgacagcaacgtgaagaacctgtacgacaaagtgcggctgcagctgagagataacgccaaggagctgggcaacggctgcttcgagttctaccacagatgcgacaacgagtgcatggagtctgtgaggaacggcacctacgactaccctcagtatagcgaggaggccaggctgaagagagaggagatcagcggcgtcaaactggagagcatcggcacctatcagatcctgagcatctacagcacagtggcctcttctctggccctggccattatggtggccggactgagcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 6120 bp, Artificial Sequence
  CMV/R Influenza A/Egypt (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccattgtgagcctggtgaagagcgaccagatctgcattggctaccacgccaacaatagcacagagcaggtggacaccatcatggagaaaaacgtgaccgtgacccacgcccaggatattctggagaaaacccacaacggcaagctgtgtgatctggacggcgtgaagcctctgatcctgagagattgtagcgtggccggatggctgctgggaaaccccatgtgcgacgagtttctgaacgtgcccgagtggagctatatcgtggagaagatcaaccccgccaacgatctgtgttaccccggcaacttcaacgattacgaggagctgaagcacctgctgtcccggatcaaccacttcgagaagatccagatcatccccaagagcagctggagcgatcacgaagccagctctggagtgtctagcgcctgtccttatcagggccggagcagcttcttcagaaacgttgtgtggctgatcaagaaggacaacgcctaccccaccatcaagcggagctacaacaacaccaaccaggaggatctgctggtcctgtggggaatccaccaccctaatgatgccgccgagcagaccagactgtaccagaaccccaccacctacatctctgtgggcaccagcaccctgaatcagagactggtgcccaagatcgccaccagatccaaggtgaacggccagagcggcagaatggagttcttctggaccatcctgaagagcaacgacgccatcaacttcgagagcaacggcaatttcatcgcccccgagaacgcctataagatcgtgaagaagggcgacagcaccatcatgaagtccgagctggagtacggcaactgcaacaccaagtgccagacacctatcggcgccatcaacagcagcatgcccttccacaacatccaccctctgaccatcggcgagtgccctaagtacgtgaagagcaacagactgatcctggccacaggcctgagaaatagccctcagagagagaccagaggcctgtttggagccatcgccggctttatcgaagggggatggcagggaatggtcgatggctggtacggctaccaccacagcaatgagcagggctctggatatgccgccgataaggagtctacccagaaggccatcgacggcgtcaccaacaaggtgaacagcatcatcgacaagatgaacacccagtttgaggctgtgggcagggagttcaacaacctggagcggaggatcgagaacctgaacaagaagatggaggacggcttcctggatgtgtggacctacaatgccgaactgctggtgctgatggagaacgagaggaccctggacttccacgacagcaacgtgaagaacctgtacgacaaagtgcggctgcagctgagagataacgccaaggagctgggcaacggctgcttcgagttctaccacagatgcgacaacgagtgcatggagtctgtgaggaacggcacctacgactaccctcagtatagcgaggaggccaggctgaagagagaggagatcagcggcgtcaaactggagagcatcggcacctatcagatcctgagcatctacagcacagtggcctcttctctggccctggccattatggtggccggactgttcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 6120 bp, Artificial Sequence
  CMV/R-Influenza A/Iraq (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccattgtgagcctggtgaagagcgaccagatctgcattggctaccacgccaacaatagcacagagcaggtggacaccatcatggagaaaaacgtgaccgtgacccacgcccaggatattctggagaaaacccacaacggcaagctgtgtgatctggacggcgtgaagcctctgatcctgagagattgtagcgtggccggatggctgctgggaaaccccatgtgcgacgagtttctgaacgtgcccgagtggagctatatcgtggagaagatcaaccccgccaacgatctgtgttaccccggcaacttcaacgattacgaggagctgaagcacctgctgtcccggatcaaccacttcgagaagatccagatcatccccaagagcagctggagcgatcacgaagccagctctggagtgtctagcgcctgtccttatcagggccggagcagcttcttcagaaacgttgtgtggctgatcaagaaggacaacgcctaccccaccatcaagcggagctacaacaacaccaaccaggaggatctgctggtcctgtggggaatccaccacccttctgatgccgccgagcagaccagactgtaccagaaccccaccacctacatctctgtgggcaccagcaccctgaatcagagactggtgcccaagatcgccaccagatccaaggtgaacggccagagcggcagaatggagttcttctggaccatcctgaagcccaacgacgccatcaacttcgagagcaacggcaatttcatcgcccccgagaacgcctataagatcgtgaagaagggcgacagcaccatcatgaagtccgagctggagtacggcaactgcaacaccaagtgccagacacctatcggcgccatcaacagcagcatgcccttccacaacatccaccctctgaccatcggcgagtgccctaagtacgtgaagagcaacagactggtgctggccacaggcctgagaaatagccctcagagagagaccagaggcctgtttggagccatcgccggctttatcgaagggggatggcagggaatggtcgatggctggtacggctaccaccacagcaatgagcagggctctggatatgccgccgataaggagtctacccagaaggccatcgacggcgtcaccaacaaggtgaacagcatcatcgacaagatgaacacccagtttgaggctgtgggcagggagttcaacaacctggagcggaggatcgagaacctgaacaagaagatggaggacggcttcctggatgtgtggacctacaatgccgaactgctggtgctgatggagaacgagaggaccctggacttccacgacagcaacgtgaagaacctgtacgacaaagtgcggctgcagctgagagataacgccaaggagctgggcaacggctgcttcgagttctaccacagatgcgacaacgagtgcatggagtctgtgaggaacggcacctacgactaccctcagtatagcgaggaggccaggctgaagagagaggagatcagcggcgtcaaactggagagcatcggcacctatcagatcctgagcatctacagcacagtggcctcttctctggccctggccattatggtggccggactgagcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 23 - DNA, 6122 bp, Artificial Sequence
  CMV/R Influenza A/Indonesia/05/05 (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgagcagaccagactgtaccagaaccccaccacctatatcagcatcggcaccagcaccctgaatcagagactggtgcccaagatcgccaccagatccaaggtgaacggccagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 6120 bp, Artificial Sequence
  CMV/R 8kb Influenza A/Indonesia/05/05-HA mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgagcagaccagactgtaccagaaccccaccacctatatcagcatcggcaccagcaccctgaatcagagactggtgcccaagatcgccaccagatccaaggtgaacggccagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 6118 bp, Artificial Sequence
  CMV/R 8kb A/Thailand/1(KAN-1)/2004 (H5N1) HA mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - DNA, 6122 bp, Artificial Sequence
  CMV/R Influenza A/Hong Kong/156/97 (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaaaacagtgctgctgctggccacagtgtctctggtgaagagcgaccagatctgtattggctaccacgccaacaattctacagagcaggtggacaccatcatggagaagaatgtgacagtgacacacgcccaggatattctggagagaacccacaatggcaagctgtgtgatctgaatggcgtgaagcctctgatcctgagagattgttctgtggccggatggctgctgggaaatcccatgtgtgacgagttcatcaatgtgcctgagtggagctatatcgtggagaaagccagccctgccaatgatctgtgttaccccggcaacttcaacgattacgaggagctgaaacacctgctgtccaggatcaatcacttcgagaagatccagatcatccctaagagcagctggtctaatcacgatgccagctctggagtgtctagcgcctgtccttatctgggcagaagcagcttcttcagaaacgtggtgtggctgatcaagaagaatagcgcctaccccacaatcaagcggagctacaacaacaccaaccaggaagatctgctggtgctgtggggaattcaccaccctaatgatgccgccgaacagacaaagctgtaccagaaccccaccacatatatctctgtgggcaccagcacactgaatcagagactggtgcctgagattgccacaagacctaaggtgaacggccagtctggcagaatggagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggccaccggactgagaaatacacctcagagagagaccagaggactgtttggagccatcgccggctttattgagggaggatggcagggaatggtggatggctggtatggctatcaccacagcaatgagcagggatctggatatgccgccgataaggagtctacacagaaggccattgacggcgtgacaaacaaggtgaacagcatcatcaacaagatgaacacccagtttgaagctgtgggcagagagttcaacaacctggagcggagaatcgagaacctgaacaagaagatggaggacggctttctggatgtgtggacctataatgccgaactgctcgtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagagcgtgaagaacggcacctacgattaccctcagtactctgaggaagccagactgaacagagaggagatctctggcgtgaaactggagagcatgggcacataccagatcctgagcatctatagcacagtggcctcttctctggccctggccattatggtggccggactgtctctgtggatgtgtagcaatggcagcctgcagtgcagaatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 27 - DNA, 6122 bp, Artificial Sequence
  CMV/R Influenza A/Hong Kong/483/97 (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccacagtgtctctggtgaagagcgaccagatctgtattggctaccacgccaacaattctacagagcaggtggacaccatcatggagaaaaacgtgacagtgacacacgcccaggatattctggagagaacccacaatggcaagctgtgtgatctgaatggcgtgaagcctctgatcctgagagattgttctgtggccggatggctgctgggaaatcccatgtgtgacgagttcatcaatgtgcctgagtggagctatatcgtggagaaagccagccctgccaatgatctgtgttaccccggcaacttcaacgattacgaggagctgaaacacctgctgtccagaatcagccacttcgagaagatccagatcatccctaagagcagctggtctaatcacgatgccagctctggagtgtctagcgcctgtccttatctgggcaagagcagcttcttcagaaatgtggtgtggctgatcaagaagaacagcacctaccccacaatcaagcggagctacaacaacaccaaccaggaagatctgctggtgctgtggggaattcaccaccctaatgatgccgccgaacagacaaagctgtaccagaaccccaccacatatatctctgtgggcaccagcacactgaatcagagactggtgcctgagattgccacaagacctaaggtgaacggccagtctggcagaatcgagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggccaccggactgagaaatgcccctcagagagagacaagaggactgtttggagccatcgccggctttattgagggaggatggcagggaatggtggatggctggtatggctatcaccacagcaatgagcagggatctggatatgccgccgatcaggagtctacacagaaagccatcgacggcgtgacaaataaggtgaacagcatcatcaacaagatgaacacccagtttgaagctgtgggcagagagttcaacaacctggagcggagaatcgagaacctgaacaagaagatggaggacggctttctggatgtgtggacctataatgccgaactgctcgtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagagcgtgaagaatggcacctacgactaccctcagtattctgaggaggccagactgaatagagaggagatcagcggcgtgaaactggaaagcatgggcacctaccagatcctgagcctgtattctacagtggcctcttctctggccctggccattatggtggccggactgtctctgtggatgtgtagcaatggcagcctgcagtgcagaatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 6122 bp, Artificial Sequence
  CMV/R Influenza A/Chicken/Korea/ES/03 (H5N1) HA-mutA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagattgtgctgctgctggccattgtgtctctggtgaagagcgaccagatctgtattggctaccacgccaacaattctacagagcaggtggacaccatcatggagaaaaacgtgacagtgacacacgcccaggatattctggagaaaacccacaatggcaagctgtgtgatctggatggagtgaagcctctgatcctgagagattgttctgtggccggatggctgctgggaaatcccatgtgtgacgagttcatcaatgtgcctgagtggagctatatcgtggagaaggccaaccctcctaatgatctgtgctaccccggcaacttcaatgattacgaggagctgaagcacctgctgtccagaatcaaccacttcgagaagatccagatcatccctaagtctagctggagcgatcacgaagcctcttctggagtgtctagcgcctgtccttatcagggcagaagcagcttcttcagaaacgtggtgtggctgatcaagaagaatagcgcctaccccacaatcaagcggagctacaacaacaccaaccaggaagatctgctggtgctgtggggaattcaccaccctaatgatgccgccgaacagacaagactgtaccagaaccccaccacatatatctctgtgggcaccagcacactgaatcagagactggtgcctaagatcgccacaagaagcaaagtgaacggccagtctggcagaatggagttcttctggaccatcctgaagccaaatgacgccatcagcttcgagagcaacggcaatttcatcgcccctgagtacgcctataagatcgtgaagaagggcgatagcgccatcatgaagagcgagctggagtacggcaactgtaataccaagtgccagacacctatgggcgccatcaatagctctatgcccttccacaatatccaccctctgacaatcggcgagtgtcctaagtacgtgaagagcagcagactggtgctggccacaggactgagaaatagccctcagagagagacaagaggactgtttggagccatcgccggctttattgagggaggatggcagggaatggtggatggctggtatggctatcaccacagcaatgagcagggatctggatatgccgccgataaggagtctacacagaaggccattgacggcgtgacaaacaaggtgaacagcatcatcgacaagatgaacacccagtttgaagctgtgggcagagagttcaacaacctggagcggagaatcgagaacctgaacaagaagatggaggacggctttctggatgtgtggacctataatgccgaactgctcgtgctgatggagaacgagagaaccctggatttccacgacagcaacgtgaagaacctgtacgacaaagtgagactgcagctgagagataatgccaaggaactgggcaatggctgcttcgagttctaccacagatgtgacaacgagtgtatcgagtctgtgagaaatggcacatacggctaccctcagtattctgaggaggccagactgaagagagaggagatctctggcgtgaagctggagagcatcggcacataccagatcctgagcatctatagcacagtggcctcttctctggccctggccattatggtggccggactgtctctgtggatgtgtagcaatggcagcctgcagtgcagaatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 29 - DNA, 6128 bp, Artificial Sequence
  CMV/R -Influenza A/swine/Ohio/51145/2007 (H1N1) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggctattctggtggtgctgctgtacacctttaccaccgccaatgccgataccctgtgtatcggctaccacgccaacaatagcaccgataccgtggacaccgtgctggaaaagaatgtgaccgtgacccactctgtgaacctgctggaaaacagacacaacggcaagctgtgtaaactgagaggtgtcgctcctctgcacctgggcaagtgtaatattgctggctggctgctgggaaatcctgagtgtgaaagcctgtctaccgccagctcttggagctacatcgtggagacaagcaacagcgataatggcacctgctaccccggcgatttcatcaactacgaggaactgagagaacagctgtccagcgtgtccagcttcgagagattcgagatcttccctaagaccagcagctggcccaatcacgataccaatagaggcgtgacagctgcttgtcctcacgccggcaccaacagcttctacagaaacctgatctggctggtcaagaagggcaacagctaccccaagatcaacaagagctacatcaacaacaaagaaaaagaggtgctggtcctctgggctatccaccatccttctacctctgcagatcagcagagcctgtatcagaatgccgacgcctacgtgtttgtgggcagcagcagatacagcagaaagttcgagcctgagatcgccacaagacctaaagtgcgggatcaggccggcagaatgaattactactggaccctggtggaacctggcgataagatcacattcgaggccactggaaatctggtggtccctagatacgccttcgccctgaagagaaatagcggcagcggcatcatcatcagcgataccagcgtgcacgattgtgataccacctgtcagacacccaatggcgccatcaataccagcctgcccttccagaatatccaccctgtgacaatcggcgagtgccctaagtacgtgaagtccaccaagctgagaatggccaccggcctgagaaatatccctagcatccagagcagaggcctgtttggagccattgccggctttattgaaggcggctggacaggcatgattgatgggtggtacggctatcaccaccagaatgagcagggatctggatatgccgccgatctgaagtctacccagaacgccatcgacggcatcaccaacaaagtgaacagcgtgatcgagaagatgaacacccagtttacagccgtgggcaaagagtttagccacctggaaagacggatcgagaacctgaacaagaaggtggacgacggcttcctggacatctggacctataatgccgaactgctggtgctgctcgagaatgagagaaccctggactaccacgacagcaacgtgaagaatctgtacgagaaagtgcggagccagctgaagaacaatgccaaagagatcggcaacggctgcttcgagttctaccacaagtgcgacgacacatgcatggaaagcgtgaagaacggcacctacgactaccctaagtacagcgaggaagccaagctgaacagagaagagatcgacggcgtgaagctggaaagcaccagaatctaccagatcctggccatctatagcacagtggcctcttctctggtgctggtcgtgtctctgggagccatcagcttttggatgtgcagcaatggcagcctgcagtgcagaatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - protein, 566 aa, Artificial Sequence
  Synthetic Construct
MKAILVVLLYTFTTANADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHAGTNSFYRNLIWLVKKGNSYPKINKSYINNKEKEVLVLWAIHHPSTSADQQSLYQNADAYVFVGSSRYSRKFEPEIATRPKVRDQAGRMNYYWTLVEPGDKITFEATGNLVVPRYAFALKRNSGSGIIISDTSVHDCDTTCQTPNGAINTSLPFQNIHPVTIGECPKYVKSTKLRMATGLRNIPSIQSRGLFGAIAGFIEGGWTGMIDGWYGYHHQNEQGSGYAADLKSTQNAIDGITNKVNSVIEKMNTQFTAVGKEFSHLERRIENLNKKVDDGFLDIWTYNAELLVLLENERTLDYHDSNVKNLYEKVRSQLKNNAKEIGNGCFEFYHKCDDTCMESVKNGTYDYPKYSEEAKLNREEIDGVKLESTRIYQILAIYSTVASSLVLVVSLGAISFWMCSNGSLQCRICI

SEQ ID NO: 31 - DNA, 6128 bp, Artificial Sequence
  CMV/R -Influenza A/swine/North Carolina/R08-001877/2008 (H3N2) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccatcattgccttcagctacatcctgtgcctgatctttgctcagaagctgcctggcagcgataattctatggccacactgtgtctgggacatcatgccgtgcctaatggcacactggtcaagaccatcaccgatgaccagatcgaagtgaccaatgccacagaactggtgcagagcagctctaccggcagaatctgtaactctccccaccagatcctggatggcaagaactgtacactgatcgatgctctgctgggagatcctcactgcgacgacttccagaacaaagaatgggacctgttcgtggagagaagcaccgcctacagcaactgttacccctactacgtgcctgattacgcctctctgagatctctggtggcctctagcggaacactggaattcacccaggaaagcttcaattggacaggcgtggctcaggatggaagcagctacgcctgtagaagaaagagcgtgaacagcttcttcagcagactgaactggctgcacaacctgaattacaagtaccccgccctgaatgtgaccatgcccaacaacgacaagttcgacaagctgtacatttggggagtgcaccaccctggaaccgatagagatcagaccaatctgtacgtgcaggcctctggaagagtgaccgtgtctaccaagagaagccagcagaccgtgatccctaatatcggatcaagaccttgggtccggggcgtgagcagcatcatcagcatctactggacaatcgtgaagcctggcgacatcctgctgatcaatagcacaggcaacctgatcgcccctagaggctactttaagatccagagcggcaagtctagcatcatgagatctgacgcccccatcggcaattgtaacagcgagtgcatcacccctaatggcagcatccccaacgacaagcccttccagaacgtgaatagaatcacctacggcgcctgtcctagatacgtgaagcagaacaccctgaaactggccacaggcatgagaaatgtgcccgagaagcagaccagaggcatttttggcgccattgccggctttatcgagaatggatgggagggaatggtggatgggtggtacggctttagacaccagaatagcgagggaacaggacaggctgccgatctgaaatctacacaggccgccgtgaatcagatcaccggcaagctgaacagagtgatcaagaaaaccaacgagaagttccaccagatcgagaaagaattcagcgaggtggagggcagaattcaggacctggaaaaatacgtggaggacaccaagatcgacctgtggagctataatgctgaactgctggtcgccctggaaaatcagcacaccatcgacctgaccgacagcgagatgaacaagctgttcgagagaaccagaaagcagctgagagaaaacgccgaggatatgggcaacggctgcttcaagatctaccacaagtgcgataatgcctgcatcggcagcatcagaaatggcacctacgaccacgatgtgtacagagatgaggccctgaacaacagatttcagatcaagggcgtgcagctgaagtctggctacaaggactggattctgtggatcagcttcgccatcagctgttttctgctgtgtgtggtgctgctgggctttattatgtgggcctgccagaaaggcaacatccggtgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 32 - protein, 566 aa, Artificial Sequence
  Synthetic Construct
MKTIIAFSYILCLIFAQKLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYASLRSLVASSGTLEFTQESFNWTGVAQDGSSYACRRKSVNSFFSRLNWLHNLNYKYPALNVTMPNNDKFDKLYIWGVHHPGTDRDQTNLYVQASGRVTVSTKRSQQTVIPNIGSRPWVRGVSSIISIYWTIVKPGDILLINSTGNLIAPRGYFKIQSGKSSIMRSDAPIGNCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAVNQITGKLNRVIKKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVQLKSGYKDWILWISFAISCFLLCVVLLGFIMWACQKGNIRCNICI

SEQ ID NO: 33 - DNA, 6120 bp, Artificial Sequence
  CMV/R -Influenza A/swine/California/04/09 (H1N1) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggctattttggtcgtgctcctgtacacctttgccacagccaatgccgataccctttgtattggctaccatgcaaacaactctaccgatacggtcgacacggtgctcgaaaagaatgttactgtcacccactctgtgaacttgctggaggataaacacaatggcaagctctgcaaactgcgaggggtggctcccctgcatctgggaaaatgtaatattgccggctggatactgggtaatccagaatgcgaatccttgagtacggcatccagttggtcctatatcgtcgagaccccgtcaagtgacaatgggacctgctacccaggcgacttcattgattatgaagagctgagggagcagttgtcatccgtaagcagcttcgaaaggtttgagattttcccgaaaactagctcctggcccaatcatgactctaacaaaggagttactgcagcctgtcctcatgcgggcgcgaaaagcttctacaagaacctgatatggctcgtgaagaaaggcaattcatacccaaaactgtctaagagctacataaacgataaagggaaagaggttctggtgctttggggcatacaccacccatctacctcagccgaccagcagtctctgtatcagaacgccgacacatacgtgtttgtgggcagctcccgctattctaagaagttcaaacccgagatcgccatcagaccaaaggtgagagaccaggaaggaaggatgaattattactggaccttggtcgaacctggcgataagataacgtttgaggctacgggcaacctggtcgtgccgagatatgcttttgccatggagaggaatgcggggagcggaattatcatcagcgacactccagttcatgactgtaataccacatgtcagacaccgaagggcgccatcaacacgagcttgccctttcagaatatacatccaatcacaatcggaaaatgccccaagtacgtgaaaagcactaaactgagactcgccaccggactcaggaatatcccaagcatccagtcacggggtctgttcggcgctatcgccggatttattgaaggcggctggacggggatggtggacggttggtacggctaccatcatcaaaatgagcagggctccggatacgccgctgacctgaaatctacgcagaatgccatagatgagatcacaaacaaggtcaatagtgtgatagaaaaaatgaatactcagttcacagctgttggaaaggagtttaaccacctcgagaagcgaattgagaacctgaacaagaaggtggacgatggctttttggatatctggacgtataacgctgagctgcttgttctgctggagaacgaaagaacccttgactaccacgattccaacgtgaagaatctgtatgagaaagtgcgaagccagttgaaaaacaacgcaaaagaaataggcaacggctgtttcgagttctaccacaaatgcgataacacctgcatggagagtgtgaagaacggaacgtacgattatccaaaatactccgaggaggccaaactcaatagggaggagatagacggtgttaagctggagtccacacgcatctatcagattctggcgatctactctactgtggcttccagcctggtgctggtcgtttcccttggggcgatcagcttctggatgtgcagcaatggctccctgcaatgccgcatctgcatctgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - protein, 566 aa, Artificial Sequence
  Synthetic Construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 35 - DNA, 6125 bp, Artificial Sequence
  CMV/R-Influenza A/equine/Ohio/1/2003 (H3N8) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccaccatcatcctgatcctgctgacccactgggcctacagccagaaccccatcagcggcaacaacaccgccaccctgtgcctgggccaccacgccgtggccaacggcaccctggtgaaaaccatctccgacgaccagatcgaggtgaccaacgccaccgagctggtgcagagcatcagcatgggcaagatctgcaacaacagctacagaatcctggacggccggaactgcaccctgatcgacgccatgctgggcgaccctcactgcgacgccttccagtacgagaactgggacctgttcatcgagcggagcagcgccttcagcaactgctacccctacgacatccccgactacgccagcctgcggagcatcgtggccagcagcggcacactggaattcaccgccgagggcttcacctggaccggcgtgacccagaacggcagaagcggcgcctgcaaacggggctccgccgatagcttcttctcccgcctcaactggctgaccaagagcggcagcagctaccccaccctgaacgtgaccatgcccaacaacaagaacttcgacaagctgtacatctggggcatccaccaccccagcagcaaccaggaacagaccaagctgtatatccaggaaagcggcagggtcaccgtgagcaccaagcggagccagcagaccatcatccccaacatcggcagccggccctgggtgcggggccagagcggccggatcagcatctactggaccatcgtgaagcccggcgacatcctgatgatcaacagcaacggcaatctggtggcccccaggggctacttcaagctgaaaaccggcaagagcagcgtgatgcggagcgacgtgcccatcgacatctgcgtgagcgagtgcatcacccccaacggcagcatcagcaacgacaagcccttccagaacgtgaacaaggtgacctacggcaagtgccccaagtacatccggcagaacaccctgaagctggccaccggcatgcggaacgtgcccgagaagcagatccggggcatcttcggcgccattgccggcttcatcgagaacggctgggagggcatggtggacggctggtatggcttcagataccagaacagcgagggcaccggccaggccgccgacctgaagagcacccaggccgccatcgaccagatcaacggcaagctgaaccgggtgatcgagcggaccaacgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacgccgagatgaacaagctgttcgagaaaaccaggcggcagctgcgggagaacgccgaggacatgggcggaggatgcttcaagatctaccacaagtgcgacaacgcctgcatcggcagcatccggaacggcacctacgaccactacatctaccgggacgaggccctgaacaaccggttccagatcaagggcgtggagctgaagagcggctacaaggactggattctgtggatcagcttcgccatcagctgctttctgatctgcgtggtgctgctgggcttcatcatgtgggcctgccagaagggcaacatccgctgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 36 - protein, 565 aa, Artificial Sequence
  Synthetic Construct
MKTTIILILLTHWAYSQNPISGNNTATLCLGHHAVANGTLVKTISDDQIEVTNATELVQSISMGKICNNSYRILDGRNCTLIDAMLGDPHCDAFQYENWDLFIERSSAFSNCYPYDIPDYASLRSIVASSGTLEFTAEGFTWTGVTQNGRSGACKRGSADSFFSRLNWLTKSGSSYPTLNVTMPNNKNFDKLYIWGIHHPSSNQEQTKLYIQESGRVTVSTKRSQQTIIPNIGSRPWVRGQSGRISIYWTIVKPGDILMINSNGNLVAPRGYFKLKTGKSSVMRSDVPIDICVSECITPNGSISNDKPFQNVNKVTYGKCPKYIRQNTLKLATGMRNVPEKQIRGIFGAIAGFIENGWEGMVDGWYGFRYQNSEGTGQAADLKSTQAAIDQINGKLNRVIERTNEKFHQIEKEFSEVEGRIQDLEKYVEDTKIDLWSYNAELLVALENQHTIDLTDAEMNKLFEKTRRQLRENAEDMGGGCFKIYHKCDNACIGSIRNGTYDHYIYRDEALNNRFQIKGVELKSGYKDWILWISFAISCFLICVVLLGFIMWACQKGNIRCNICI

SEQ ID NO: 37 - DNA, 6131 bp, Artificial Sequence
  CMV/R-Influenza A/equine/Bari/2005 (H3N8) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccaccatcatcttcatctttatcctgctgacccactgggcctacagccagaaccccatcagcgacaacaacaccgccaccctgtgcctgggccaccacgccgtggccaacggcaccctggtgaaaaccatctccgacgaccagatcgaggtgaccaacgccaccgagctggtgcagagcatcagcatgggcaagatctgcaacaacagctacagaatcctggacggccggaactgcaccctgatcgacgccatgctgggcgaccctcactgcgacgtgttccagtacgagaactgggacctgttcatcgagcggagcagcgccttcagcaactgctacccctacgacatccccgactacgccagcctgcggagcatcgtggccagcagcggcacactggaattcaccgccgagggcttcacctggaccggcgtgacccagaacggcagaagcggcgcctgcaaacggggctccgccgatagcttcttctcccgcctcaactggctgaccaagagcggcaacagctaccccaccctgaacgtgaccatgcccaacaacaagaacttcgacaagctgtacatctggggcatccaccaccccagcagcaaccaggaacagaccaagctgtatatccaggaaagcggcagggtcaccgtgagcaccaagcggagccagcagaccatgatccccaacatcggcagccggccctgggtgcggggccagagcggccggatcagcatctactggaccatcgtgaagcccggcgacatcctgatgatcaacagcaacggcaatctggtggcccccaggggctacttcaagctgaaaaccggcaagagcagcgtgatgcggagcgacgtgcccatcgacatctgcgtgagcgagtgcatcacccccaacggcagcatcagcaacgacaagcccttccagaacgtgaacaaggtgacctacggcaagtgccccaagtacatccggcagaacaccctgaagctggccaccggcatgcggaacgtgcccgagaagcagatccggggcatcttcggcgccattgccggcttcatcgagaacggctgggagggcatggtggacggctggtatggcttcagataccagaacagcgagggcaccggccaggccgccgacctgaagagcacccaggccgccatcgaccagatcaacggcaagctgaaccgggtgatcgagcggaccaacgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacgccgagatgaacaagctgttcgagaaaaccaggcggcagctgcgggagaacgccgaggacatgggcggaggatgcttcaagatctaccacaagtgcgacaacgcctgcatcggcagcatccggaacggcacctacgaccactacatctaccgggacgaggccctgaacaaccggttccagatcaagggcgtggagctgaagagcggctacaaggactggattctgtggatcagcttcgccatcagctgctttctgatctgcgtggtgctgctgggcttcatcatgtgggcctgccagaagggcaacatccgctgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - protein, 567 aa, Artificial Sequence
  Synthetic Construct
MKTTIIFIFILLTHWAYSQNPISDNNTATLCLGHHAVANGTLVKTISDDQIEVTNATELVQSISMGKICNNSYRILDGRNCTLIDAMLGDPHCDVFQYENWDLFIERSSAFSNCYPYDIPDYASLRSIVASSGTLEFTAEGFTWTGVTQNGRSGACKRGSADSFFSRLNWLTKSGNSYPTLNVTMPNNKNFDKLYIWGIHHPSSNQEQTKLYIQESGRVTVSTKRSQQTMIPNIGSRPWVRGQSGRISIYWTIVKPGDILMINSNGNLVAPRGYFKLKTGKSSVMRSDVPIDICVSECITPNGSISNDKPFQNVNKVTYGKCPKYIRQNTLKLATGMRNVPEKQIRGIFGAIAGFIENGWEGMVDGWYGFRYQNSEGTGQAADLKSTQAAIDQINGKLNRVIERTNEKFHQIEKEFSEVEGRIQDLEKYVEDTKIDLWSYNAELLVALENQHTIDLTDAEMNKLFEKTRRQLRENAEDMGGGCFKIYHKCDNACIGSIRNGTYDHYIYRDEALNNRFQIKGVELKSGYKDWILWISFAISCFLICVVLLGFIMWACQKGNIRCNICI

SEQ ID NO: 39 - DNA, 6125 bp, Artificial Sequence
  CMV/R-Influenza A/equine/Aboyne/1/05(H3N8) HA/h
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggaaaccaccatcatcctgatcctgctgacccactgggtgtacagccagaaccccatcagcggcaacaacaccgccacactgtgtctgggacaccacgccgtggccaacggaaccctggtgaaaaccatcaccgacgaccagatcgaagtgaccaacgccaccgaactggtggagagcatcagcatgggcaagatctgcaacaacagctaccgggtgctggacggcagaaactgcaccctgatcgacgccatgctgggcgaccctcactgcgacgacttccagtacgagagctgggacctgttcatcgagcggagcagcgccagcagcaactgctacccctacgacatccccgactacgccagcctgagaagcatcgtggccagcagcggcacactggaattcaccgccgagggcttcacctggaccggcgtgacccagaatggcagaagcggcgcctgcaagagaggcagcgccgacagcttcttctcaagactgaactggctgaccaagagcggcaactcctaccccaccctgaacgtgaccatgcccaacaacaagaacttcgacaagctgtacatctggggcatccaccaccccagctccaacaaagagcagaccaagctgtatatccaggaatccggcagagtcaccgtgtccaccgagagaagccagcagaccgtgatccccaacatcggcagcagaccttgggtgcgcggccagagcggcagaatcagcatctactggaccatcgtgaagcccggcgacgtgctgatgatcaacagcaacggcaatctggtggcccccagaggctacttcaagctgcggaccggcaagagcagcgtgatgcggagcgacgccctgatcgatacctgcgtgagcgagtgcatcacccccaacggcagcatccccaacgacaagcccttccagaacgtgaacaagatcacctacggccggtgccccaagtacatccggcagaacaccctgaagctggccaccggcatgagaaacgtgcccgagaagcagatcagaggcatcttcggcgccattgccggcttcatcgagaacggctgggagggcatggtggacgggtggtacggcttcagataccagaacagcgagggcacaggacaggccgccgacctgaagtctacccaggccgccatcgaccagatcaacggcaagctgaacagagtgatcgagcggaccaacgagaagttccaccagatcgagaaagaattcagcgaggtggagggcagaatccaggacctggaaaaatacgtggaggacaccaagatcgacctgtggagctacaacgccgagctgctggtcgccctggaaaaccagcacaccatcgacctgaccgacgccgagatgaacaagctgttcgagagaaccagacggcagctgcgggagaacgccgaggatatgggcggaggatgcttcaagatctaccacaagtgcgacaacgcctgcatcggctccatccggaacggcacctacgaccactacatctaccgggacgaggccctgaacaaccggttccagatcaagggcgtggagctgaagtccggctacaaggactggattctgtggatcagcttcgccatcagctgctttctgatctgcgtggtgctgctgggcttcatcatgtgggcctgccagaagggcaacatccgctgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 40 - protein, 565 aa, Artificial Sequence
  Synthetic Construct
METTIILILLTHWVYSQNPISGNNTATLCLGHHAVANGTLVKTITDDQIEVTNATELVESISMGKICNNSYRVLDGRNCTLIDAMLGDPHCDDFQYESWDLFIERSSASSNCYPYDIPDYASLRSIVASSGTLEFTAEGFTWTGVTQNGRSGACKRGSADSFFSRLNWLTKSGNSYPTLNVTMPNNKNFDKLYIWGIHHPSSNKEQTKLYIQESGRVTVSTERSQQTVIPNIGSRPWVRGQSGRISIYWTIVKPGDVLMINSNGNLVAPRGYFKLRTGKSSVMRSDALIDTCVSECITPNGSIPNDKPFQNVNKITYGRCPKYIRQNTLKLATGMRNVPEKQIRGIFGAIAGFIENGWEGMVDGWYGFRYQNSEGTGQAADLKSTQAAIDQINGKLNRVIERTNEKFHQIEKEFSEVEGRIQDLEKYVEDTKIDLWSYNAELLVALENQHTIDLTDAEMNKLFERTRRQLRENAEDMGGGCFKIYHKCDNACIGSIRNGTYDHYIYRDEALNNRFQIKGVELKSGYKDWILWISFAISCFLICVVLLGFIMWACQKGNIRCNICI

SEQ ID NO: 41 - DNA, 4414 bp, Artificial Sequence
  CMV/R
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 42 - DNA, 1698 bp, Artificial Sequence
  Hemagglutinin coding sequence from SEQ ID NO29
atgaaggctattctggtggtgctgctgtacacctttaccaccgccaatgccgataccctgtgtatcggctaccacgccaacaatagcaccgataccgtggacaccgtgctggaaaagaatgtgaccgtgacccactctgtgaacctgctggaaaacagacacaacggcaagctgtgtaaactgagaggtgtcgctcctctgcacctgggcaagtgtaatattgctggctggctgctgggaaatcctgagtgtgaaagcctgtctaccgccagctcttggagctacatcgtggagacaagcaacagcgataatggcacctgctaccccggcgatttcatcaactacgaggaactgagagaacagctgtccagcgtgtccagcttcgagagattcgagatcttccctaagaccagcagctggcccaatcacgataccaatagaggcgtgacagctgcttgtcctcacgccggcaccaacagcttctacagaaacctgatctggctggtcaagaagggcaacagctaccccaagatcaacaagagctacatcaacaacaaagaaaaagaggtgctggtcctctgggctatccaccatccttctacctctgcagatcagcagagcctgtatcagaatgccgacgcctacgtgtttgtgggcagcagcagatacagcagaaagttcgagcctgagatcgccacaagacctaaagtgcgggatcaggccggcagaatgaattactactggaccctggtggaacctggcgataagatcacattcgaggccactggaaatctggtggtccctagatacgccttcgccctgaagagaaatagcggcagcggcatcatcatcagcgataccagcgtgcacgattgtgataccacctgtcagacacccaatggcgccatcaataccagcctgcccttccagaatatccaccctgtgacaatcggcgagtgccctaagtacgtgaagtccaccaagctgagaatggccaccggcctgagaaatatccctagcatccagagcagaggcctgtttggagccattgccggctttattgaaggcggctggacaggcatgattgatgggtggtacggctatcaccaccagaatgagcagggatctggatatgccgccgatctgaagtctacccagaacgccatcgacggcatcaccaacaaagtgaacagcgtgatcgagaagatgaacacccagtttacagccgtgggcaaagagtttagccacctggaaagacggatcgagaacctgaacaagaaggtggacgacggcttcctggacatctggacctataatgccgaactgctggtgctgctcgagaatgagagaaccctggactaccacgacagcaacgtgaagaatctgtacgagaaagtgcggagccagctgaagaacaatgccaaagagatcggcaacggctgcttcgagttctaccacaagtgcgacgacacatgcatggaaagcgtgaagaacggcacctacgactaccctaagtacagcgaggaagccaagctgaacagagaagagatcgacggcgtgaagctggaaagcaccagaatctaccagatcctggccatctatagcacagtggcctcttctctggtgctggtcgtgtctctgggagccatcagcttttggatgtgcagcaatggcagcctgcagtgcagaatctgtatc

SEQ ID NO: 43 - DNA, 1698 bp, Artificial Sequence
  Hemagglutinin coding sequence from SEQ ID NO31
atgaaaaccatcattgccttcagctacatcctgtgcctgatctttgctcagaagctgcctggcagcgataattctatggccacactgtgtctgggacatcatgccgtgcctaatggcacactggtcaagaccatcaccgatgaccagatcgaagtgaccaatgccacagaactggtgcagagcagctctaccggcagaatctgtaactctccccaccagatcctggatggcaagaactgtacactgatcgatgctctgctgggagatcctcactgcgacgacttccagaacaaagaatgggacctgttcgtggagagaagcaccgcctacagcaactgttacccctactacgtgcctgattacgcctctctgagatctctggtggcctctagcggaacactggaattcacccaggaaagcttcaattggacaggcgtggctcaggatggaagcagctacgcctgtagaagaaagagcgtgaacagcttcttcagcagactgaactggctgcacaacctgaattacaagtaccccgccctgaatgtgaccatgcccaacaacgacaagttcgacaagctgtacatttggggagtgcaccaccctggaaccgatagagatcagaccaatctgtacgtgcaggcctctggaagagtgaccgtgtctaccaagagaagccagcagaccgtgatccctaatatcggatcaagaccttgggtccggggcgtgagcagcatcatcagcatctactggacaatcgtgaagcctggcgacatcctgctgatcaatagcacaggcaacctgatcgcccctagaggctactttaagatccagagcggcaagtctagcatcatgagatctgacgcccccatcggcaattgtaacagcgagtgcatcacccctaatggcagcatccccaacgacaagcccttccagaacgtgaatagaatcacctacggcgcctgtcctagatacgtgaagcagaacaccctgaaactggccacaggcatgagaaatgtgcccgagaagcagaccagaggcatttttggcgccattgccggctttatcgagaatggatgggagggaatggtggatgggtggtacggctttagacaccagaatagcgagggaacaggacaggctgccgatctgaaatctacacaggccgccgtgaatcagatcaccggcaagctgaacagagtgatcaagaaaaccaacgagaagttccaccagatcgagaaagaattcagcgaggtggagggcagaattcaggacctggaaaaatacgtggaggacaccaagatcgacctgtggagctataatgctgaactgctggtcgccctggaaaatcagcacaccatcgacctgaccgacagcgagatgaacaagctgttcgagagaaccagaaagcagctgagagaaaacgccgaggatatgggcaacggctgcttcaagatctaccacaagtgcgataatgcctgcatcggcagcatcagaaatggcacctacgaccacgatgtgtacagagatgaggccctgaacaacagatttcagatcaagggcgtgcagctgaagtctggctacaaggactggattctgtggatcagcttcgccatcagctgttttctgctgtgtgtggtgctgctgggctttattatgtgggcctgccagaaaggcaacatccggtgcaacatctgcatc

SEQ ID NO: 44 - DNA, 1698 bp, Artificial Sequence
  Hemagglutinin coding sequence from SEQ ID NO33
atgaaggctattttggtcgtgctcctgtacacctttgccacagccaatgccgataccctttgtattggctaccatgcaaacaactctaccgatacggtcgacacggtgctcgaaaagaatgttactgtcacccactctgtgaacttgctggaggataaacacaatggcaagctctgcaaactgcgaggggtggctcccctgcatctgggaaaatgtaatattgccggctggatactgggtaatccagaatgcgaatccttgagtacggcatccagttggtcctatatcgtcgagaccccgtcaagtgacaatgggacctgctacccaggcgacttcattgattatgaagagctgagggagcagttgtcatccgtaagcagcttcgaaaggtttgagattttcccgaaaactagctcctggcccaatcatgactctaacaaaggagttactgcagcctgtcctcatgcgggcgcgaaaagcttctacaagaacctgatatggctcgtgaagaaaggcaattcatacccaaaactgtctaagagctacataaacgataaagggaaagaggttctggtgctttggggcatacaccacccatctacctcagccgaccagcagtctctgtatcagaacgccgacacatacgtgtttgtgggcagctcccgctattctaagaagttcaaacccgagatcgccatcagaccaaaggtgagagaccaggaaggaaggatgaattattactggaccttggtcgaacctggcgataagataacgtttgaggctacgggcaacctggtcgtgccgagatatgcttttgccatggagaggaatgcggggagcggaattatcatcagcgacactccagttcatgactgtaataccacatgtcagacaccgaagggcgccatcaacacgagcttgccctttcagaatatacatccaatcacaatcggaaaatgccccaagtacgtgaaaagcactaaactgagactcgccaccggactcaggaatatcccaagcatccagtcacggggtctgttcggcgctatcgccggatttattgaaggcggctggacggggatggtggacggttggtacggctaccatcatcaaaatgagcagggctccggatacgccgctgacctgaaatctacgcagaatgccatagatgagatcacaaacaaggtcaatagtgtgatagaaaaaatgaatactcagttcacagctgttggaaaggagtttaaccacctcgagaagcgaattgagaacctgaacaagaaggtggacgatggctttttggatatctggacgtataacgctgagctgcttgttctgctggagaacgaaagaacccttgactaccacgattccaacgtgaagaatctgtatgagaaagtgcgaagccagttgaaaaacaacgcaaaagaaataggcaacggctgtttcgagttctaccacaaatgcgataacacctgcatggagagtgtgaagaacggaacgtacgattatccaaaatactccgaggaggccaaactcaatagggaggagatagacggtgttaagctggagtccacacgcatctatcagattctggcgatctactctactgtggcttccagcctggtgctggtcgtttcccttggggcgatcagcttctggatgtgcagcaatggctccctgcaatgccgcatctgcatc

SEQ ID NO: 45 - DNA, 1695 bp, Artificial Sequence
  Hemagglutinin coding sequence from SEQ ID NO35
atgaaaaccaccatcatcctgatcctgctgacccactgggcctacagccagaaccccatcagcggcaacaacaccgccaccctgtgcctgggccaccacgccgtggccaacggcaccctggtgaaaaccatctccgacgaccagatcgaggtgaccaacgccaccgagctggtgcagagcatcagcatgggcaagatctgcaacaacagctacagaatcctggacggccggaactgcaccctgatcgacgccatgctgggcgaccctcactgcgacgccttccagtacgagaactgggacctgttcatcgagcggagcagcgccttcagcaactgctacccctacgacatccccgactacgccagcctgcggagcatcgtggccagcagcggcacactggaattcaccgccgagggcttcacctggaccggcgtgacccagaacggcagaagcggcgcctgcaaacggggctccgccgatagcttcttctcccgcctcaactggctgaccaagagcggcagcagctaccccaccctgaacgtgaccatgcccaacaacaagaacttcgacaagctgtacatctggggcatccaccaccccagcagcaaccaggaacagaccaagctgtatatccaggaaagcggcagggtcaccgtgagcaccaagcggagccagcagaccatcatccccaacatcggcagccggccctgggtgcggggccagagcggccggatcagcatctactggaccatcgtgaagcccggcgacatcctgatgatcaacagcaacggcaatctggtggcccccaggggctacttcaagctgaaaaccggcaagagcagcgtgatgcggagcgacgtgcccatcgacatctgcgtgagcgagtgcatcacccccaacggcagcatcagcaacgacaagcccttccagaacgtgaacaaggtgacctacggcaagtgccccaagtacatccggcagaacaccctgaagctggccaccggcatgcggaacgtgcccgagaagcagatccggggcatcttcggcgccattgccggcttcatcgagaacggctgggagggcatggtggacggctggtatggcttcagataccagaacagcgagggcaccggccaggccgccgacctgaagagcacccaggccgccatcgaccagatcaacggcaagctgaaccgggtgatcgagcggaccaacgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacgccgagatgaacaagctgttcgagaaaaccaggcggcagctgcgggagaacgccgaggacatgggcggaggatgcttcaagatctaccacaagtgcgacaacgcctgcatcggcagcatccggaacggcacctacgaccactacatctaccgggacgaggccctgaacaaccggttccagatcaagggcgtggagctgaagagcggctacaaggactggattctgtggatcagcttcgccatcagctgctttctgatctgcgtggtgctgctgggcttcatcatgtgggcctgccagaagggcaacatccgctgcaacatctgcatc

SEQ ID NO: 46 - DNA, 1701 bp, Artificial Sequence
  Hemagglutinin coding sequence from SEQ ID NO37
atgaaaaccaccatcatcttcatctttatcctgctgacccactgggcctacagccagaaccccatcagcgacaacaacaccgccaccctgtgcctgggccaccacgccgtggccaacggcaccctggtgaaaaccatctccgacgaccagatcgaggtgaccaacgccaccgagctggtgcagagcatcagcatgggcaagatctgcaacaacagctacagaatcctggacggccggaactgcaccctgatcgacgccatgctgggcgaccctcactgcgacgtgttccagtacgagaactgggacctgttcatcgagcggagcagcgccttcagcaactgctacccctacgacatccccgactacgccagcctgcggagcatcgtggccagcagcggcacactggaattcaccgccgagggcttcacctggaccggcgtgacccagaacggcagaagcggcgcctgcaaacggggctccgccgatagcttcttctcccgcctcaactggctgaccaagagcggcaacagctaccccaccctgaacgtgaccatgcccaacaacaagaacttcgacaagctgtacatctggggcatccaccaccccagcagcaaccaggaacagaccaagctgtatatccaggaaagcggcagggtcaccgtgagcaccaagcggagccagcagaccatgatccccaacatcggcagccggccctgggtgcggggccagagcggccggatcagcatctactggaccatcgtgaagcccggcgacatcctgatgatcaacagcaacggcaatctggtggcccccaggggctacttcaagctgaaaaccggcaagagcagcgtgatgcggagcgacgtgcccatcgacatctgcgtgagcgagtgcatcacccccaacggcagcatcagcaacgacaagcccttccagaacgtgaacaaggtgacctacggcaagtgccccaagtacatccggcagaacaccctgaagctggccaccggcatgcggaacgtgcccgagaagcagatccggggcatcttcggcgccattgccggcttcatcgagaacggctgggagggcatggtggacggctggtatggcttcagataccagaacagcgagggcaccggccaggccgccgacctgaagagcacccaggccgccatcgaccagatcaacggcaagctgaaccgggtgatcgagcggaccaacgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacgccgagatgaacaagctgttcgagaaaaccaggcggcagctgcgggagaacgccgaggacatgggcggaggatgcttcaagatctaccacaagtgcgacaacgcctgcatcggcagcatccggaacggcacctacgaccactacatctaccgggacgaggccctgaacaaccggttccagatcaagggcgtggagctgaagagcggctacaaggactggattctgtggatcagcttcgccatcagctgctttctgatctgcgtggtgctgctgggcttcatcatgtgggcctgccagaagggcaacatccgctgcaacatctgcatc

SEQ ID NO: 47 - DNA, 1695 bp, Artificial Sequence
  Hemagglutinin coding sequence from SEQ ID NO39
atggaaaccaccatcatcctgatcctgctgacccactgggtgtacagccagaaccccatcagcggcaacaacaccgccacactgtgtctgggacaccacgccgtggccaacggaaccctggtgaaaaccatcaccgacgaccagatcgaagtgaccaacgccaccgaactggtggagagcatcagcatgggcaagatctgcaacaacagctaccgggtgctggacggcagaaactgcaccctgatcgacgccatgctgggcgaccctcactgcgacgacttccagtacgagagctgggacctgttcatcgagcggagcagcgccagcagcaactgctacccctacgacatccccgactacgccagcctgagaagcatcgtggccagcagcggcacactggaattcaccgccgagggcttcacctggaccggcgtgacccagaatggcagaagcggcgcctgcaagagaggcagcgccgacagcttcttctcaagactgaactggctgaccaagagcggcaactcctaccccaccctgaacgtgaccatgcccaacaacaagaacttcgacaagctgtacatctggggcatccaccaccccagctccaacaaagagcagaccaagctgtatatccaggaatccggcagagtcaccgtgtccaccgagagaagccagcagaccgtgatccccaacatcggcagcagaccttgggtgcgcggccagagcggcagaatcagcatctactggaccatcgtgaagcccggcgacgtgctgatgatcaacagcaacggcaatctggtggcccccagaggctacttcaagctgcggaccggcaagagcagcgtgatgcggagcgacgccctgatcgatacctgcgtgagcgagtgcatcacccccaacggcagcatccccaacgacaagcccttccagaacgtgaacaagatcacctacggccggtgccccaagtacatccggcagaacaccctgaagctggccaccggcatgagaaacgtgcccgagaagcagatcagaggcatcttcggcgccattgccggcttcatcgagaacggctgggagggcatggtggacgggtggtacggcttcagataccagaacagcgagggcacaggacaggccgccgacctgaagtctacccaggccgccatcgaccagatcaacggcaagctgaacagagtgatcgagcggaccaacgagaagttccaccagatcgagaaagaattcagcgaggtggagggcagaatccaggacctggaaaaatacgtggaggacaccaagatcgacctgtggagctacaacgccgagctgctggtcgccctggaaaaccagcacaccatcgacctgaccgacgccgagatgaacaagctgttcgagagaaccagacggcagctgcgggagaacgccgaggatatgggcggaggatgcttcaagatctaccacaagtgcgacaacgcctgcatcggctccatccggaacggcacctacgaccactacatctaccgggacgaggccctgaacaaccggttccagatcaagggcgtggagctgaagtccggctacaaggactggattctgtggatcagcttcgccatcagctgctttctgatctgcgtggtgctgctgggcttcatcatgtgggcctgccagaagggcaacatccgctgcaacatctgcatc